(19) United States
(12) Patent Application Publication
(10) Pub. No.: US 2021/0018515 A1
(43) Pub. Date: Jan. 21, 2021

(54) BIOMARKER COMBINATION FOR IDENTIFICATION OF "AT-RISK" SUBJECTS FOR AKI

(71) Applicants: RANDOX LABORATORIES LIMITED, Crumlin, Antrim (GB); BELFAST HEALTH AND SOCIAL CARE TRUST, Belfast (GB)

(72) Inventors: Mary Jo KURTH, Crumlin, Antrim (GB); John LAMONT, Crumlin, Antrim (GB); Peter FITZGERALD, Crumlin, Antrim (GB); Mark RUDDOCK, Crumlin, Antrim (GB); William MCBRIDE, Belfast, Antrim (GB)

(21) Appl. No.: 17/040,016

(22) PCT Filed: Mar. 22, 2019
(86) PCT No.: PCT/GB2019/050835

§ 371 Date: Sep. 21, 2020

(30) Foreign Application Priority Data
Mar. 22, 2018 (GB) 1804648.2

(51) Int. Cl. G01N 33/68

(52) U.S. Cl. CPC G01N 33/6893; G01N 2800/347; G01N 2800/50; G01N 2800/60; G01N 2333/475; G01N 2800/52; G01N 2333/70578; G01N 2333/5434; G01N 2333/521; G01N 2333/7155; G01N 2333/4704

(57) ABSTRACT

A method for determining predisposition of a subject to developing renal dysfunction (AKI), and to a kit for use in making such a determination is provided. Suitably at least one marker selected from Midkine (MK) or H-FABP present in a blood or urine sample is used in the method.

The present invention relates to a method for determining predisposition of a subject to developing renal dysfunction, and to a kit for use in making such a determination.

Acute Kidney Injury (AKI), is a recognised complication of cardiac surgery (CS) with cardiopulmonary bypass (CPB) and is characterised by an abrupt deterioration of estimated glomular filtration rate (eGFR). AKI-CS is associated with increased hospital stay, morbidity and mortality. Identification and diagnosis of AKI-CS is based on changes in serum creatinine concentration. However, this is problematic as increases in serum creatinine may be delayed by 24-72 hours post insult. Early prediction and identification of ‘at-risk’ patients would allow implementation of suitable preoperative, intraoperative and postoperative renal protection strategies. The benefits of early Renal Replacement Therapy (RRT), compared to late RRT, are associated with improved patient survival and decreased length of ICU stay (RRT<3 days' post-surgery).

Pre and intra-operative renal protection strategies, such as administration of erythropoietin or minimising the duration of CPB may be provided to those subjects identified pre-surgery to be at risk of AKI. Post-surgery detection of those subjects at risk from AKI, suitably within 24 hours from surgery, would allow early post-operative interventions such as Renal Replacement Therapy.

Further, subjects suffering physical trauma, which can include fractures, often develop acute renal dysfunction (AKI). Other causes of a similar acute renal dysfunction include prolonged hypotensive states (e.g. associated with mucosal gut ischaemia and endotoxin translocation from gut to circulation), sepsis and septic shock syndromes.

A robust post physical trauma test, suitably within 24 hours of the trauma, would allow preventative measures to be taken in the intensive care unit with subjects considered to be ‘at risk’ of AKI.

### SUMMARY OF THE INVENTION

Whilst several markers have been identified for identification of subjects ‘at risk’ of developing Acute Kidney Injury (AKI) improved detection of such subjects would be beneficial.

The inventors have determined that measurement of specific combinations of biomarkers present in a subject pre and post trauma can provide for improved detection of subjects ‘at risk’ of developing AKI.

Accordingly, a first aspect of the present invention provides a method to determine a predisposition of a subject to developing AKI, the method comprising the step of:

determining the level of at least one marker selected from Midkine (MK) or H-FABP present in a blood or urine sample taken from the subject prior to surgery, physical trauma, hypotension, sepsis and/or septic shock syndrome, in particular cardiac surgery or a fracture trauma;

wherein when the level of the Midkine or H-FABP is higher than a normal level of Midkine or H-FABP in a blood or urine sample from a control it is indicative the subject has a greater than normal predisposition for developing AKI following surgery, physical trauma, hypotension, sepsis and/or septic shock syndrome, in particular cardiac surgery or a fracture trauma.

In one aspect, the present disclosure provides methods of diagnosing or aiding in the diagnosis of AKI in a subject, comprising: analyzing a biological sample from said subject to determine the level(s) of one or more biomarkers for AKI in the sample, where the one or more biomarkers are selected from Tables 2, 3, 4, 5, 6 or 7 and comparing the level(s) of the one or more biomarkers in the sample to AKI-positive and/or AKI-negative reference levels of the one or more biomarkers in order to diagnose whether the subject has AKI.

According to a second aspect of the present invention there is provided a method to determine a predisposition of a subject to developing AKI, the method comprising the step of:

determining the level of at least one marker selected from Midkine (MK) or H-FABP present in a blood or urine sample taken from the subject within 48 hours following surgery, physical trauma, hypotension, sepsis and/or septic shock syndrome, in particular cardiac surgery or a fracture trauma;

wherein when the level of the Midkine or H-FABP is higher than a normal level of Midkine or H-FABP in a blood or urine sample from a control it is indicative the subject has a greater than normal predisposition for developing AKI following surgery, physical trauma, hypotension, sepsis and/or septic shock syndrome, in particular cardiac surgery or a fracture trauma.

As used herein, marker and biomarker are used interchangeably.

As will be appreciated, the level(s) of biomarker(s) from a subject being tested may be compared with the level(s) of biomarker(s) in a sample from a subject with an AKI disease state and when the level(s) of the biomarker(s) in the sample from the subject being tested is not differential to the level(s) of biomarker(s) in the sample from the subject with an AKI disease state, it is indicative that the subject being tested has AKI.

Alternatively, as will be appreciated, the level(s) of biomarker(s) from a subject being tested may be compared with the level(s) of biomarker(s) in a sample from a subject with a non AKI disease state and when the level(s) of the biomarker(s) in the sample from the subject being tested is not differential to the level(s) of biomarker(s) in the sample from the subject with a non AKI disease state, it is indicative that the subject being tested does not have AKI.

“Biomarker” means a biological compound, that is differentially present (i.e. increased or decreased) in a biological sample from a subject or a group of subjects having a first phenotype (e.g. having a disease) as compared to a biological sample from a subject or group of subjects having a second phenotype (e.g. not having the disease). A biomarker may be differentially present at any level, but suitably differentially present at a level that is statistically significant (i.e, a p-value less than 0.05 and/or a q-value of less than 0.10 as determined using either Welch's T-test or Wilcoxon's rank-sum Test). Suitably the biomarker may be present at a level that has an AUC (area under the curve of a receiving operating characteristic curve) of 0.7 or greater, suitably 0.75 or greater, suitably 0.8 or greater. The “level” of one or more biomarkers means the absolute or relative amount or concentration of the biomarker in the sample.

“Sample” or “biological sample” means biological material isolated from a subject. The biological sample may contain any biological material suitable for detecting the desired biomarkers, and may comprise cellular and/or non-cellular material from the subject. The sample can be isolated from any suitable biological fluid such as, for example, blood, blood plasma, blood serum, or urine.

A “reference level or normal level” of a biomarker means a level of the biomarker that is indicative of a non AKI state, phenotype, or predisposition to developing an AKI disease state or phenotype. A “level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate normal reference levels of biomarkers for AKI may be determined by measuring levels of desired biomarkers in one or more appropriate subjects, and such reference levels may be tailored to specific populations of subjects (e.g., a reference level may be age-matched or gender-matched so that comparisons may be made between biomarker levels in samples from subjects of a certain age or gender and reference levels for a AKI disease state, phenotype, or lack thereof in a certain age or gender group).

When the methods of the invention are used to aid in the diagnosis of AKI, the results of the method may be used along with other methods (or the results thereof) useful in the clinical determination of whether a subject has AKI.

Any suitable method may be used to analyze the biological sample in order to determine the level(s) of the one or more biomarkers in the sample. Suitable methods include chromatography (e.g., HPLC, gas chromatography, liquid chromatography), mass spectrometry (e.g., MS, MS-MS), enzyme-linked immunosorbent assay (ELISA), antibody linkage, other immunochemical techniques, and combinations thereof. Further, the level(s) of the one or more biomarkers may be measured indirectly, for example, by using an assay that measures the level of a compound (or compounds) that correlates with the level of the biomarker(s) that are desired to be measured.

It will be appreciated that the markers as discussed herein may be detected using any suitable assays or devices, for example ELISA assay, BIOCHIP technology or the like. Suitably, TNFRI and II biomarkers (Soluble Tumour Necrosis Factor Receptors I and II) may be detected using a Cytokine Array IV (Randox).

Suitably, determining levels of combinations of the biomarkers may allow greater sensitivity and specificity in diagnosing AKI and aiding in the diagnosis of AKI. For example, ratios of the levels of certain biomarkers (and non-biomarker compounds, for example metabolites) in biological samples may allow greater sensitivity and specificity in diagnosing AKI and aiding in the diagnosis of AKI. After the level(s) of the one or more biomarkers in the sample are determined, the level(s) can be compared to AKI-positive and/or AKI-negative reference levels to diagnose or aid in diagnosing whether the subject has AKI. Levels of the one or more biomarkers in a sample matching the AKI-positive reference levels (e.g., levels that are the same as the reference levels, substantially the same as the reference levels, above and/or below the minimum and/or maximum of the reference levels, and/or within the range of the reference levels) are indicative of a diagnosis of AKI in the subject. Levels of the one or more biomarkers in a sample matching the AKI-negative reference levels (e.g., levels that are the same as the reference levels, substantially the same as the reference levels, above and/or below the minimum and/or maximum of the reference levels, and/or within the range of the reference levels) are indicative of a diagnosis of no AKI in the subject. In addition, levels of the one or more biomarkers that are differentially present (especially at a level that is statistically significant) in the sample as compared to AKI-negative reference levels are indicative of a diagnosis of AKI in the subject. Levels of the one or more biomarkers that are differentially present (especially at a level that is statistically significant) in the sample as compared AKI-positive reference levels are indicative of a diagnosis of no AKI in the subject.

The level(s) of the one or more biomarkers may be compared to AKI-positive and/or AKI-negative reference levels using various techniques, including a simple comparison (e.g., a manual comparison) of the level(s) of the one or more biomarkers in the biological sample to AKI-positive and/or AKI-negative reference levels. The level(s) of the one or more biomarkers in the biological sample may also be compared to AKI-positive and/or AKI-negative reference levels using one or more statistical analyses (e.g., t-test, Welch's T-test, VVilcoxon's rank sum test, Random Forest, T-score, Z-score) or using a mathematical model (e.g., algorithm, statistical model, mixed effects model).

Suitably Midkine (MK or NEGF-2—a 13 kDa heparin-binding embryonic growth factor may be detected using an ELISA assay as available from Cellmid (Australia). Midkine is a known marker in relation to cancer (see for example: Muramatsu, T., Midkine and pleiotrophin: two related proteins involved in development, survival, inflammation and tumorigenesis. J Biochem, 2002. 132(3): p.359-71;. Ikematsu, S., et al., Serum midkine levels are increased in patients with various types of carcinomas. Br J Cancer, 2000. 83(6): p. 701-6; Ibusuki, M., et al., Midkine in plasma as a novel breast cancer marker. Cancer Sci,2009. 100(9): p. 1735-9; Ikematsu, S., et al., Correlation of elevated level of blood midkine with poor prognostic factors of human neuroblastomas. Br J Cancer, 2003. 88(10): p. 1522-6.)

The inventors have determined that detecting the markers TNFRI, TNFRII and Midkine in a pre-operative serum sample from a subject in a model for AKI, an area under the curve of a receiving operating characteristic curve for a sample pre-surgery from a subject to undergo cardiac surgery with cardiopulmonary bypass is of about 0.75. Further, the inventors have determined that using the markers TNFRI, TNFRII and Midkine in a post-operative serum sample from a subject, an area under the curve of a receiving operating characteristic curve for a sample post-surgery from a subject who has undergone cardiac surgery with cardiopulmonary bypass is of about 0.80.

Suitably the inventors have determined that using the markers TNFRI and TNFRII in a pre-operative serum sample from a subject, an area under the curve of a receiving operating characteristic curve for a sample pre-surgery from a subject to undergo cardiac surgery with cardiopulmonary bypass is of about 0.73.

Suitably the inventors have determined that using the markers TNFRI and Midkine in a pre-operative serum sample from a subject, an area under the curve of a receiving operating characteristic curve for a sample pre-surgery from a subject to undergo cardiac surgery with cardiopulmonary bypass is of about 0.74.

Suitably the inventors have determined that using the markers TNFRII and Midkine in a pre-operative serum sample from a subject, an area under the curve of a receiving operating characteristic curve for a sample pre-surgery from a subject to undergo cardiac surgery with cardiopulmonary bypass is of about 0.75.

Further, the inventors have determined that using the markers TNFRI, TNFRII and Midkine in a post-operative serum sample from a subject, an area under the curve of a receiving operating characteristic curve for a sample post-surgery from a subject who has undergone cardiac surgery with cardiopulmonary bypass is of about 0.80.

Suitably the inventors have determined that using the markers TNFRI and TNFRII in a post-operative serum sample from a subject, an area under the curve of a receiving operating characteristic curve for a sample post-surgery from a subject who has undergone cardiac surgery with cardiopulmonary bypass is of about 0.77.

Suitably the inventors have determined that using the markers TNFRI and Midkine in a post-operative serum sample from a subject, an area under the curve of a receiving operating characteristic curve for a sample post-surgery from a subject who has undergone cardiac surgery with cardiopulmonary bypass is of about 0.76.

Suitably the inventors have determined that using the markers TNFRII and Midkine in a post-operative serum sample from a subject, an area under the curve of a receiving operating characteristic curve for a sample post-surgery from a subject who has undergone cardiac surgery with cardiopulmonary bypass is of about 0.80.

Suitably the inventors have determined that using the markers TNFRI and H-FABP in a pre-operative serum sample from a subject, an area under the curve of a receiving operating characteristic curve for a sample pre-surgery from a subject to undergo a fracture trauma is of about 0.74.

Suitably the inventors have determined that using the markers TNFRI and H-FABP in a post-operative serum sample from a subject, an area under the curve of a receiving operating characteristic curve for a sample post-surgery from a subject who has undergone fracture trauma is of about 0.87.

Suitably the inventors have determined that using the markers TNFRI and H-FABP and Midkine in a pre-operative serum sample from a subject, an area under the curve of a receiving operating characteristic curve for a sample pre-surgery from a subject to undergo a fracture trauma is of about 0.76.

Suitably the inventors have determined that using the markers TNFRI and H-FABP and Midkine in a post-operative serum sample from a subject, an area under the curve of a receiving operating characteristic curve for a sample post-surgery from a subject who has undergone fracture trauma is of about 0.88.

After the level(s) of the one or more biomarker(s) is determined, the level(s) may be compared to AKI disease or non AKI disease reference level(s) or reference curves of the one or more biomarker(s) to determine a rating for each of the one or more biomarker(s) in the sample. The rating(s) may be aggregated using any algorithm to create a score, for example, an AKI score, for the subject. The algorithm may take into account any factors relating to AKI including the number of biomarkers, the correlation of the biomarkers to AKI, etc. Suitably, a mathematical model or formula containing one or more biomarkers as variables may be established using regression analysis, e.g., multiple linear regressions. By way of non-limiting example, the developed formulas may include the following:

A+B(Biomarker₁)+C(Biomarker₂)+D(Biomarker₃)+E(Biomarker₄)=PatientScore

A+B*In(Biomarker₁)+C*In(Biomarker₂)+D*In(Biomarker₃)+E*In(Biomarker₄)=InPatient Score

wherein A, B, C, D, E can be constant numbers; Biomarker₁, Biomarker₂, Biomarker₃, Biomarker₄ are the measured values of a respective Biomarker and PatientScore is the measure of AKI presence or absence or severity.

Suitably a pre-operative patient score can be provided which is indicative the subject has a greater than normal predisposition for developing AKI following cardiac surgery with cardiopulmonary bypass.

Suitably a cardiac patient score may be provided by, for example,

7(log₁₀ TNFRII+1)+2(log₁₀TNFRI+1)+⅓(log₁₀MK+1)

In an embodiment when a patient score is greater than or equal to 2.6 it is indicative the subject is at risk of AKI. In an embodiment when a patient score is greater than or equal to 2.7 and less than 3.4 it is indicative the subject is at risk of AKI. In an embodiment when a patient score is greater than or equal to 2.75 and less than 3.3 it is indicative the subject is at risk of AKI. In an embodiment when a patient score is greater than or equal to 2.9 and less than 3.1 it is indicative the subject is at risk of AKI.

Suitably, in relation to fracture repair, the subjects undergo two insults. The initial insult and the surgery to repair the fracture. The time delay between the injury occurring and the surgery, results in subjects becoming conditioned i.e. the inflammatory process has started before surgery. As a result, the subjects may develop AKI before surgery. The classification of AKI was based on the RIFLE system. Patients were assumed to have a baseline GFR of at least 60 ml/min/1.73 m² therefore, a value of less than 45 ml/min/1.73 m²was used to define a patient as AKI positive.

Suitably a pre-fracture repair patient score may be provided by, for example,

1.579(log₁₀(H-FABP+1))+8.555(log₁₀TNFRI+1)

In an embodiment when a patient score is greater than or equal to 4 it is indicative the subject is at risk of AKI. In an embodiment when a patient score is greater than or equal to 4.1 and less than 5 it is indicative the subject is at risk of AKI. In an embodiment when a patient score is greater than or equal to 4.2 and less than 4.5 it is indicative the subject is at risk of AKI. In an embodiment when a patient score is greater than or equal to 4.3 and less than 4.5 it is indicative the subject is at risk of AKI.

Suitably, samples for analysis are taken prior to surgery and within 24 hours following surgery. Subjects with a MDRD<45 ml/min/1.73 m² at any time were defined as AKI positive.

In embodiments of the second aspect of the present invention a sample may be taken from the subject within 24 hours following surgery, physical trauma, hypotension, sepsis and/or septic shock syndrome, in particular cardiac surgery or a fracture trauma wherein when the level of TNFRI, TNFRII and Midkine is higher than a normal level of TNFRI, TNFRII and Midkine in a sample it is indicative the subject has a greater than normal predisposition for developing AKI following cardiac surgery with cardiopulmonary bypass.

The inventors have determined that using the markers TNFRI, TNFRII and Midkine in a post-operative sample from a subject, a patient score can be provided which is indicative the subject has a greater than normal predisposition for developing AKI following cardiac surgery with cardiopulmonary bypass.

Suitably a post operative cardiac patient score may be provided by, for example,

7.3(log₁₀ TNFRII)+2(log₁₀TNFRI)+6/5(log₁₀MK)

In an embodiment when a patient score is greater than or equal to 6.8 it is indicative the subject is at risk of AKI. In an embodiment when a patient score is greater than or equal to 6.9 and less than 7.6 it is indicative the subject is at risk of AKI. In an embodiment when a patient score is greater than or equal to 6.9 and less than 7.5 it is indicative the subject is at risk of AKI. In an embodiment when a patient score is greater than or equal to 7.0 and less than 7.4 it is indicative the subject is at risk of AKI.

The inventors have determined that using the markers TNFRI and H-FABP in a post-operative sample from a subject, a patient score can be provided which is indicative the subject has a greater than normal predisposition for developing AKI following cardiac surgery with cardiopulmonary bypass.

Suitably a post-fracture patient score may be provided by, for example,

4.81 (log₁₀(H-FABP+1))+9.7(log₁₀TNFRI+1)

In an embodiment when a patient score is greater than or equal to 10.5 it is indicative the subject is at risk of AKI. In an embodiment when a patient score is greater than or equal to 10.7 and less than 14 it is indicative the subject is at risk of AKI. In an embodiment when a patient score is greater than or equal to 10.8 and less than 14 it is indicative the subject is at risk of AKI. In an embodiment when a patient score is greater than or equal to 11 and less than 14 it is indicative the subject is at risk of AKI.

In an embodiment the patient score is determined from a sample obtained from a subject within 24 hours of a surgery. In an embodiment the patient score is determined from a sample obtained from a subject within 48 hours of a surgery. In an embodiment the patient score is determined from a sample obtained from a subject within 72 hours of a surgery. In an embodiment the patient score is determined from a sample obtained from a subject within 120 hours of a surgery. Suitably a sample from a subject may be obtained within 24 to 48 hours post surgery, suitably within 48 hours to 72 hours post surgery, suitably within 72 to 120 hours post surgery.

In embodiments the sample for use in the first or second aspect can be from blood. Suitably the sample may be plasma or serum. In embodiments the sample can be urine.

Suitably, the method may allow determination of a predisposition of renal dysfunction wherein the renal dysfunction is acute renal dysfunction. Suitably, the renal dysfunction may present 5-days or more following surgery, physical trauma, hypotension, sepsis and/or septic shock syndrome, in particular cardiac surgery or a fracture trauma

Suitably, renal dysfunction may be defined as a 25% or more decrease from normal glomerular filtration rate (eGFR less than 75%).

The method of the present invention predicts the likelihood of the subject developing post-event (i.e. post surgery, physical trauma, hypotension, sepsis and/or septic shock syndrome, in particular cardiac surgery or a fracture trauma) renal dysfunction, should those events occur. The method is therefore a prognostic method. Subjects may be determined to have a greater than normal chance of developing renal dysfunction when they present with a patient score derived from a combination of TNFRI, TNFRII and MK in a serum sample greater than a normal patient score derived from such a combination.

A ‘normal or control level’ (non AKI) patient score is the level presented by a control group of individuals who did not develop AKI.

The renal dysfunction to be prognosed may be early renal dysfunction, late renal dysfunction, or general renal dysfunction. Early renal dysfunction occurs within two days of the event that induces the renal dysfunction. Late renal dysfunction occurs 5 days or later after such an event. Determining whether or not an individual has renal dysfunction is a clinical question well within the abilities of the skilled person. However, in the interests of clarity, renal dysfunction is characterised by a reduction in the capacity to excrete metabolic products which accumulate systemically and are detectable clinicopathologically by renal function tests (in progressed states, renal dysfunction may be acute kidney failure, uremia or chronic renal damage). For example, renal dysfunction may be defined as a 25% or more decrease from normal glomerular filtration rate. Normal glomerular filtration rate is the pre-event rate. Glomerular filtration rate may be established in accordance with the MDRD study group formula. Such acute forms of renal dysfunction can be distinguished from autoimmune mediated chronic renal dysfunction, a condition that is clinically apparent over a prolonged period of time in parallel with the coexisting autoimmune condition (i.e. there is no requirement for a biological marker to predict the development of renal dysfunction occurring a few days later because the renal dysfunction is already well established).

The sample taken from the subject may be any sample capable of being analysed for the level of anti-inflammatory cytokine therein. For example, the sample may be a urine sample, a blood sample, for example a serum or a plasma sample. A serum sample is particularly preferred.

The samples analysed according to the first aspect of the invention may be obtained from the subject 48, 24 or 12 hours before the event (i.e. cardiac surgery). The sample will optimally be obtained 24 hours before the event.

In embodiment of the first or second aspect the step of determining the level of at least one marker selected from Midkine or H-FABP can be undertaken on serum from the subject.

In embodiments of the first or second aspect the step of determining the level comprises determining the level of at least two markers wherein a first marker is selected from at least one of Midkine (MK) and H-FABP and at least a second marker is selected from at least one of TNFRI and TNFRII wherein when the detected level of TNFRI and/or TNFRII is higher than a normal level of TNFRI and/or TNFRII respectively in a control it is indicative the subject has a greater than normal predisposition for developing AKI following cardiac surgery or fracture trauma.

In embodiments TNFRI and/or TNFRII can be detected in a urine sample and Midkine and/or H-FABP can be detected in serum. Suitably a ratio of TNFRI and/or TNFRII and Midkine and/or H-FABP may be determined.

In embodiments the determining step can comprise determining the level of at least three markers selected from TNFRI, TNFRII and Midkine.

In embodiments the determining step can comprise determining the level of at least three markers selected from TNFRI, TNFRII and H-FABP.

In embodiments the determining step can comprise determining the level of at least one marker selected from TNFRI, H-FABP and Midkine. Suitably the methods may include detection of an additional marker selected from at least one of IL-1a, IL-5, IL-6, IL-8, IL-10, IL-15, VEGF, INF-gamma, TNF-alpha, MCP, MIP1-alpha, NGAL, IL12P40, IP10 or IL1Ra. Suitably, at least one of IL-1a, IL-5, IL-6, IL-8, IL-10, IL-15, VEGF, INF-gamma, TNF-alpha, MCP, MIP1-alpha and NGAL may be detected from plasma from the subject. Suitably, IL12P40 may be detected from serum or urine of a subject. Suitably, IP10 or IL1Ra may be detected in serum or urine. Suitably NGAL may be detected in urine.

Suitably when considering a subject who will undergo cardiac surgery the following markers may be utilized in a diagnostic model IL-6, IL-1a, VEGF, INF-gamma, TNF-alpha, MCP, MIP1-alpha and NGAL.

Suitably when considering a subject who will or has undergone cardiac surgery a post surgery model may include, TNF-alpha, MCP, MIP1-alpha and NGAL.

In embodiments where more than one marker is measured, the individual markers may be measured in samples obtained at the same time, or they may be determined from samples obtained at different times (e.g., an earlier or later time). The individual markers may also be measured in the same or different body fluid samples. For example, one marker may be measured in a serum or plasma sample and another marker may be measured in a urine sample. In addition to absolute levels, ratios of markers may be useful in identifying subjects at risk of AKI within the scope of the current invention. For example, the ratio of one marker measured in serum to another marker measured in urine, at the same or different time points, may be informative to the prognosis of AKI following cardiac or orthopedic surgery. Suitable ratios would include, for example, H-FABP to TNFRI, Midkine to TNFRI, H-FABP to Midkine, H-FABP to TNFRII, Midkine to TNFRII and TNFRI to TNFRII.

In a third aspect of the present invention, there is provided a kit for use in the method of the first or second aspect, wherein the kit comprises:-
one or more reagents to detect at least one of TNFI, TNFII, H-FABP and Midkine or a combination thereof
instructions for determining whether the at least one of TNFRI, TNFRII, H-FABP and Midkine is higher than a normal level as observed in a subject according to the first or second aspect.

The kit may further comprise one or more reagents for the detection of one or more anti-inflammatory mediator, for example an anti-inflammatory cytokine, and instructions for using the one or more reagents for detecting the one or more anti-inflammatory mediator.

The kit may further comprise instructions for using the detection of the one or more anti-inflammatory cytokines in order to arrive at a prognosis for renal dysfunction. The instructions may be in accordance with the steps for prognosis of renal dysfunction provided in the first or second aspect of the present invention. The kit may further comprise instructions for using the detecting of the one or more anti-inflammatory mediator in order to arrive at a prognosis for renal dysfunction.

In a fourth aspect of the present invention there is provided a method of treating renal dysfunction induced by surgery, physical trauma, hypotension, sepsis and/or septic shock syndrome, in particular cardiac surgery or a fracture trauma, wherein the method includes the steps of: (i) prognosing renal dysfunction according to any of the methods of the first aspect or second aspect of the present invention; and (ii) when the subject is identified to be at increased risk of developing renal dysfunction, applying therapeutic measures to treat or obviate the impending renal dysfunction.

The advantage of such a method over current therapeutic interventions is that therapy may be administered at a stage when full renal failure may be prevented. The therapeutic measures applied in step (ii) may be: maintaining a supra-normal blood pressure; ensuring adequate tissue oxygen delivery; administration of steroids; renal replacement therapy; dialysis; or any combination thereof, administration of erythropoietin, minimizing the duration of cardiopulmonary bypass, early renal replacement therapy. A further advantage of this invention would be to allow intensive care managers to identify early in the intensive care stay of the patient those individuals who are likely to spend longer in intensive care than would otherwise be anticipated providing earlier planning for staff deployment. The biomarker(s) and algorithm(s) discussed herein may assist a physician to decide a treatment path.

In embodiments a physical trauma can be the impact on the body from external forces applied to the body, for example:- lesions caused by surgery or by blows or cuts to the body (such as might be experienced during a car crash), or; the impact on the blood as it interacts with the foreign surface of a heart-lung bypass machine. Renal dysfunction induced by physical trauma may be postoperative renal dysfunction. The post-operative renal dysfunction may be following cardiac, cardiovascular, cardiopulmonary or bone fracture surgery. Physical trauma does not include reperfusion injury.

Whether or not an individual has hypotension is a clinical question and therefore well within the skill of an ordinary person in the art. For the avoidance of doubt however hypotension in adults may be defined as a systolic blood pressure <80 mmHg, or a mean arterial pressure (MAP)<50 mmHg. The hypotension may be prolonged, for example for over 2 hours.

Whether or not an individual has sepsis is a clinical question and therefore well within the skill of an ordinary person in the art. For the avoidance of doubt however, sepsis may be considered present if infection is highly suspected or proven and two or more of the following systemic inflammatory response syndrome (SIRS) criteria are met:
1. Heart rate >90 beats per minute (tachycardia);
2. Body temperature <36° C. (97° F.) or >38° C. (100° F.) (hypothermia or fever);
3. Respiratory rate >20 breaths per minute or, on blood gas, a PₐCO₂ less than 32 mm Hg (4.3 kPa) (tachypnea or hypocapnia due to hyperventilation); and
4. White blood cell count <4,000 cells/mm³ or >12,000 cells/mm³ (<4×10⁹ or >12×10⁹ cells/L), or greater than 10% band forms.

Whether or not an individual has septic shock is a clinical question and therefore well within the abilities of an ordinary person skilled in the art. For the avoidance of doubt however, septic shock may be defined by the presence of the following two criteria:
1. Evidence of infection, through a positive blood culture; and
2. Refractory hypotension—hypotension despite adequate fluid resuscitation and cardiac output. In adults it is defined as a systolic blood pressure <90 mmHg, or a MAP <60 mmHg, before institution of required resuscitative inotropic support, or a reduction of 40 mmHg in the systolic blood pressure from baseline. In children it is BP <2 SD of the normal blood pressure.

Whether or not an individual has SIRS is a clinical question and therefore well within the abilities of an ordinary person skilled in the art. For the avoidance of doubt however SIRS may be diagnosed when two or more of the following are present:
1. Heart rate >90 beats per minute
2. Body temperature <36 or >38° C.
3. Tachypnea (high respiratory rate) >20 breaths per minute or, on blood gas, a PₐCO₂<4.3 kPa (32 mm Hg)
4. White blood cell count <4000 cells/mm³ or >12000 cells/mm³ (<4×10⁹ or >12×10⁹ cells/L), or the presence of greater than 10% immature neutrophils.

Preferred features and embodiments of each aspect of the invention are as for each of the other aspects mutatis mutandis unless context demands otherwise.

As used herein, the articles “a” and “an” refer to one or to more than one (for example to at least one) of the grammatical object of the article.

“About” shall generally mean an acceptable degree of error for the quantity measured given the nature or precision of the measurements.

Throughout the specification, unless the context demands otherwise, the terms ‘comprise’ or ‘include’, or variations such as ‘comprises’ or ‘comprising’, ‘includes’ or ‘including’ will be understood to imply the includes of a stated integer or group of integers, but not the exclusion of any other integer or group of integers.

Embodiments of the present invention will now be described, by way of example, with reference to the accompanying figures, in which:-

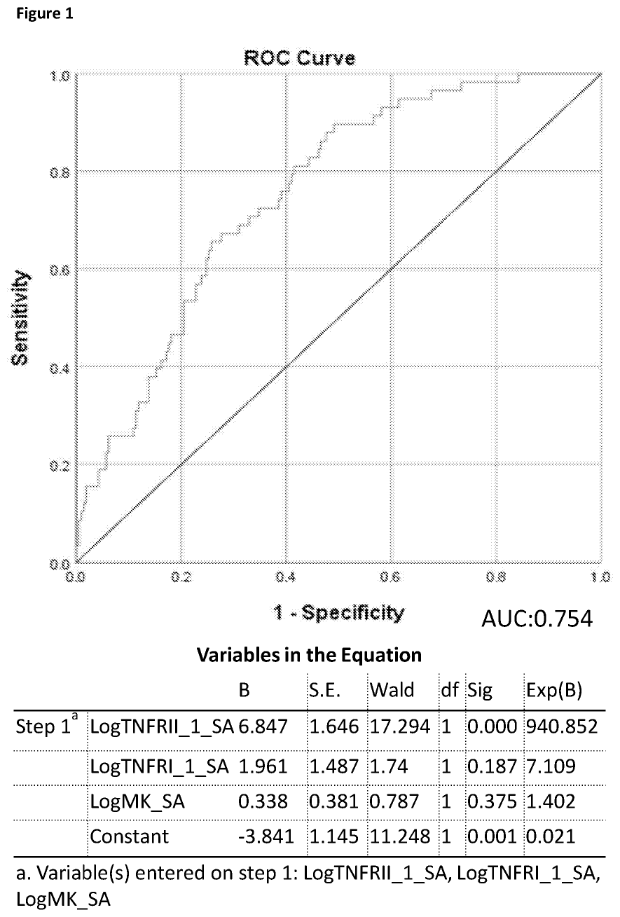

FIG. 1 shows pre-surgery ROC of TNFRI, TNFRII and Midkine model;

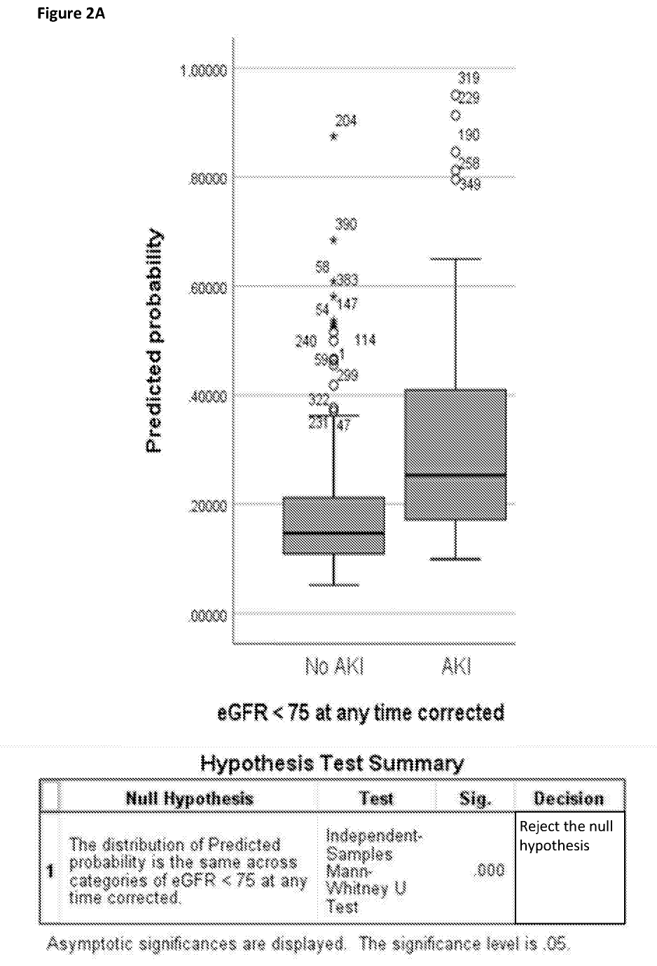

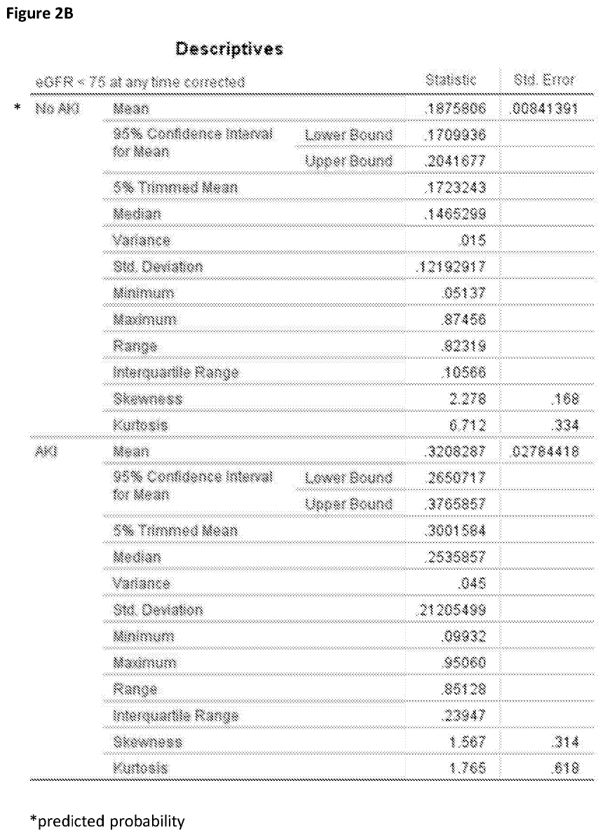

FIG. 2 shows a pre-surgery TNFRI, TNFRII and Midkine model;

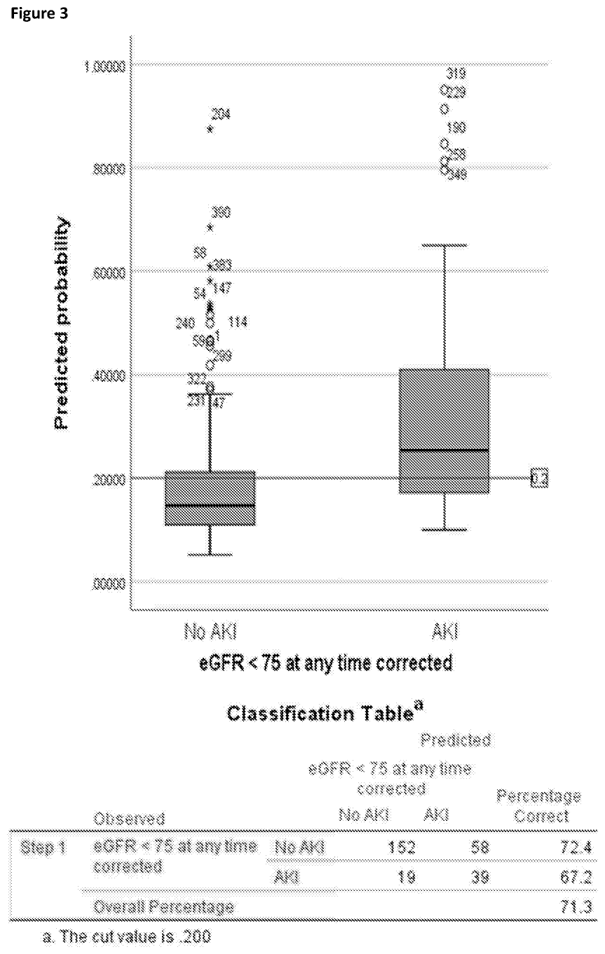

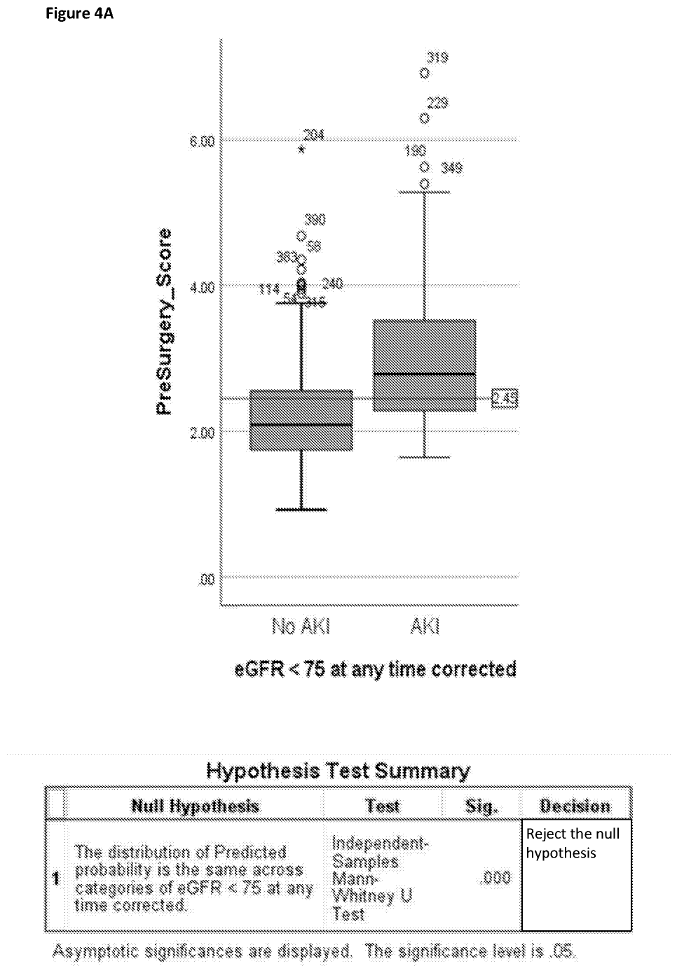

FIG. 3 shows a pre-surgery TNFRI, TNFRII and Midkine model;

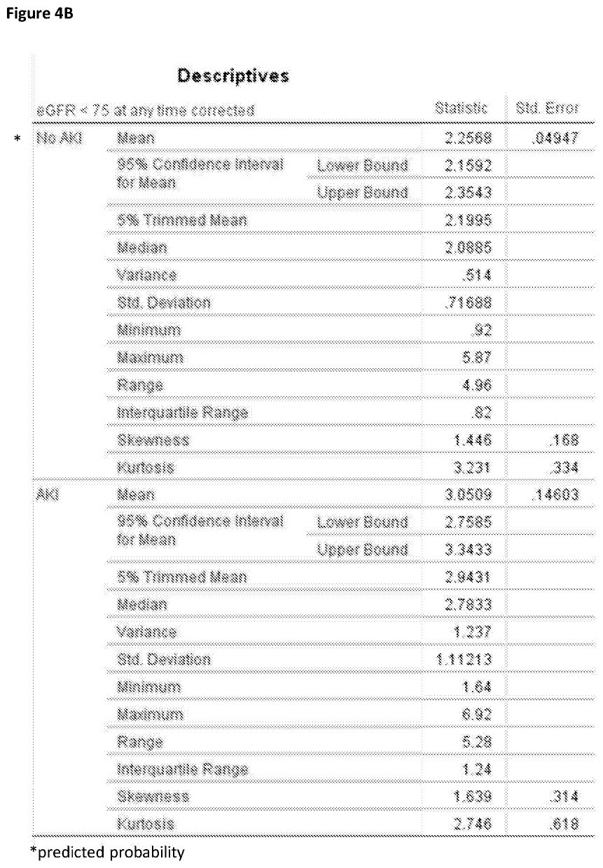

FIG. 4 shows a patient scores using Serum Pre-surgery TNFRI, TNFRII and Midkine model;

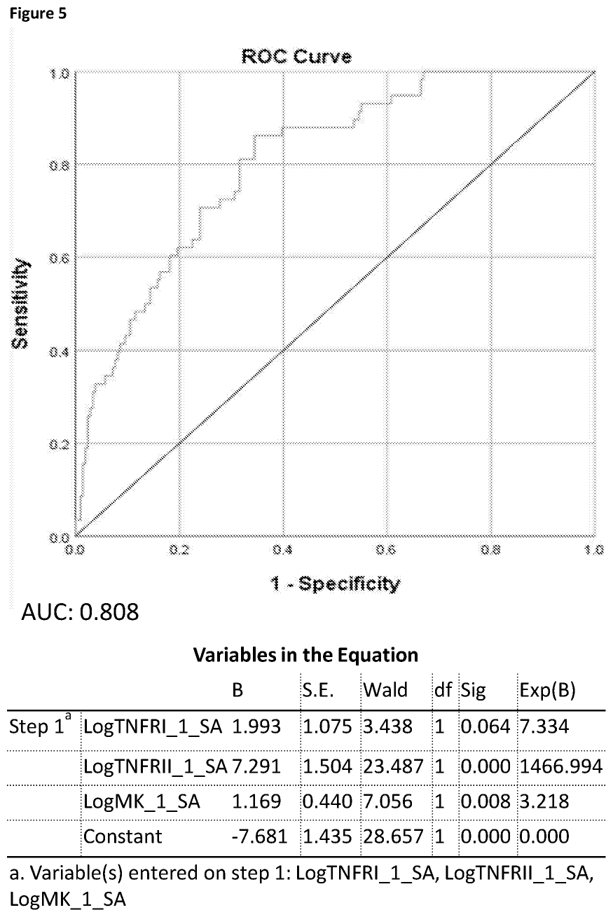

FIG. 5 shows ROC for a serum post surgery TNFRI, TNFRII and Midkine model;

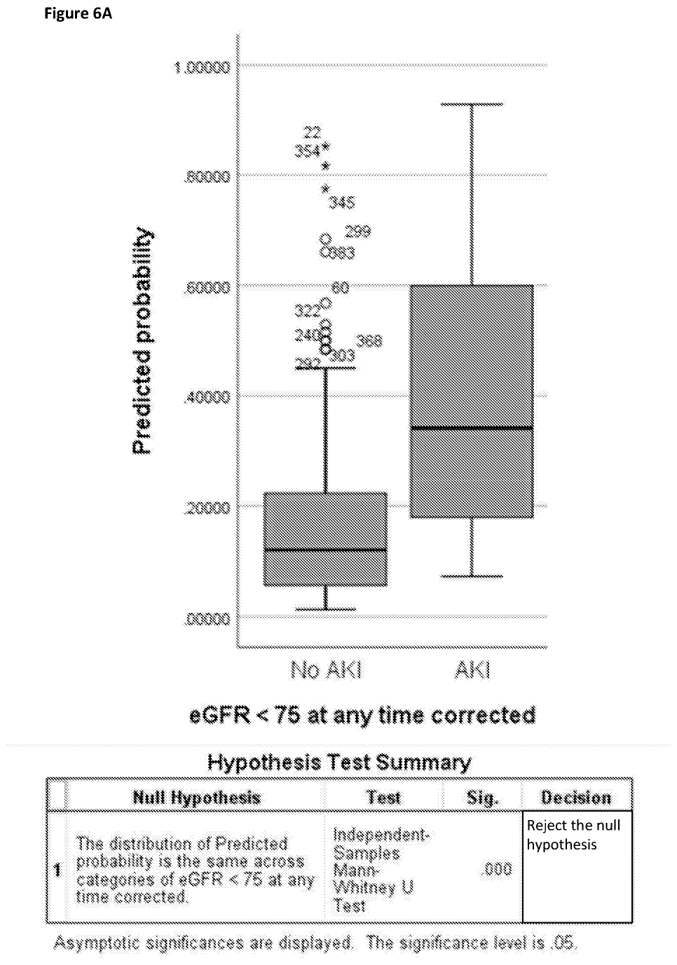

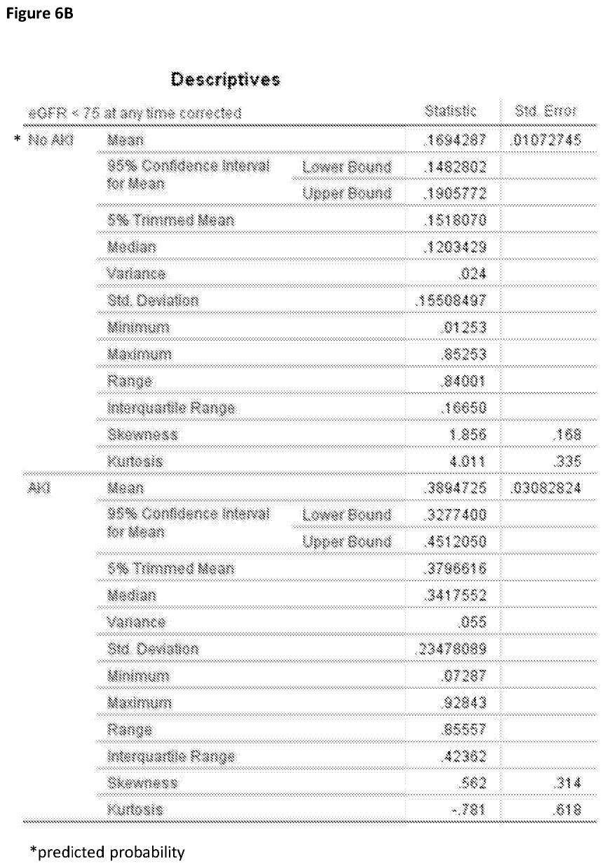

FIG. 6 shows a serum post surgery TNFRI, TNFRII and Midkine model;

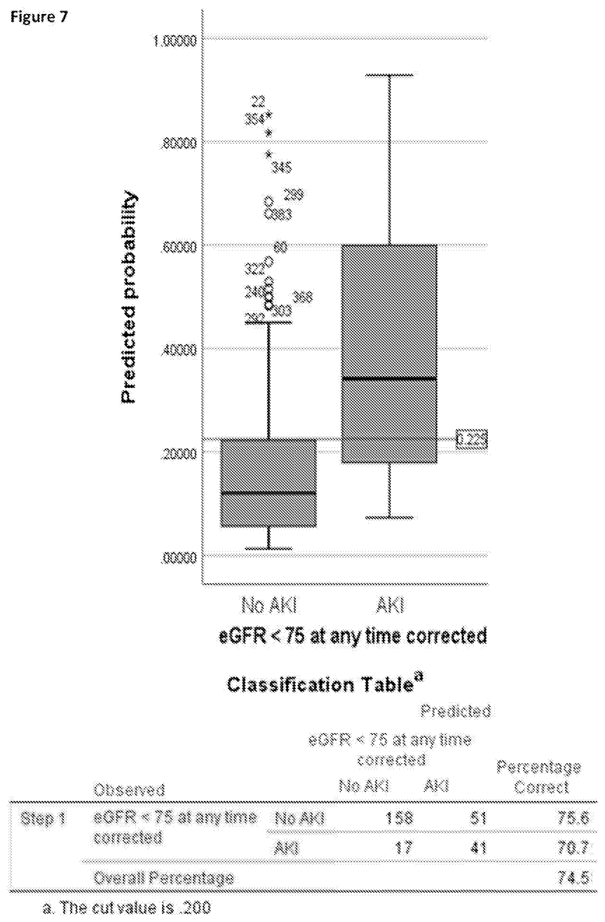

FIG. 7 shows a serum post surgery TNFRI, TNFRII and Midkine model;

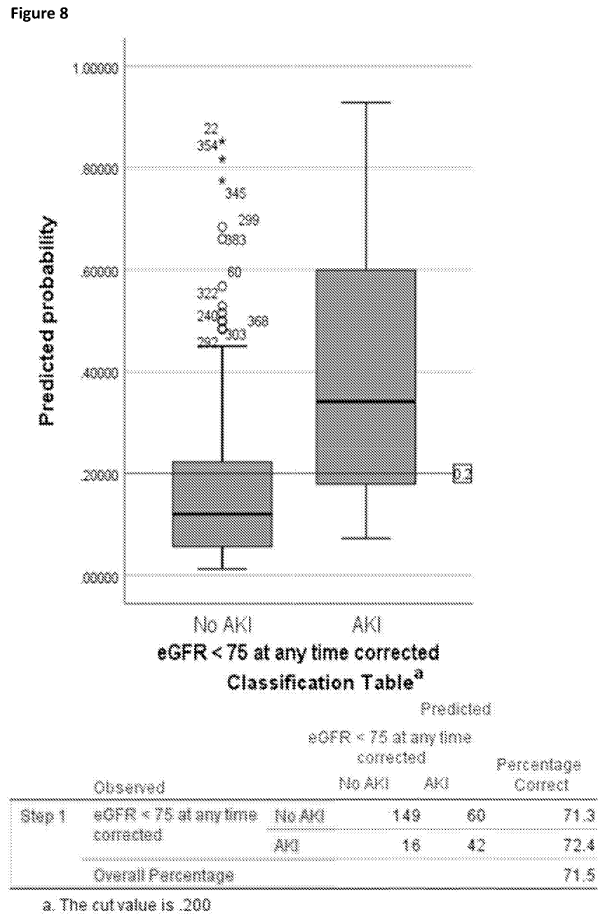

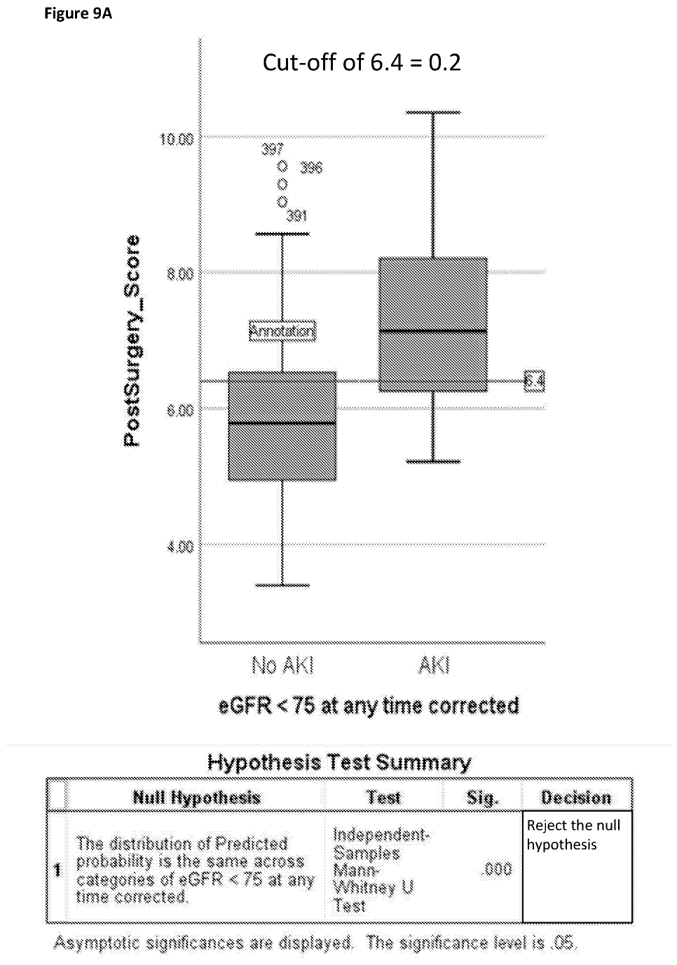

FIG. 8 shows a serum post surgery TNFRI, TNFRII and Midkine model;

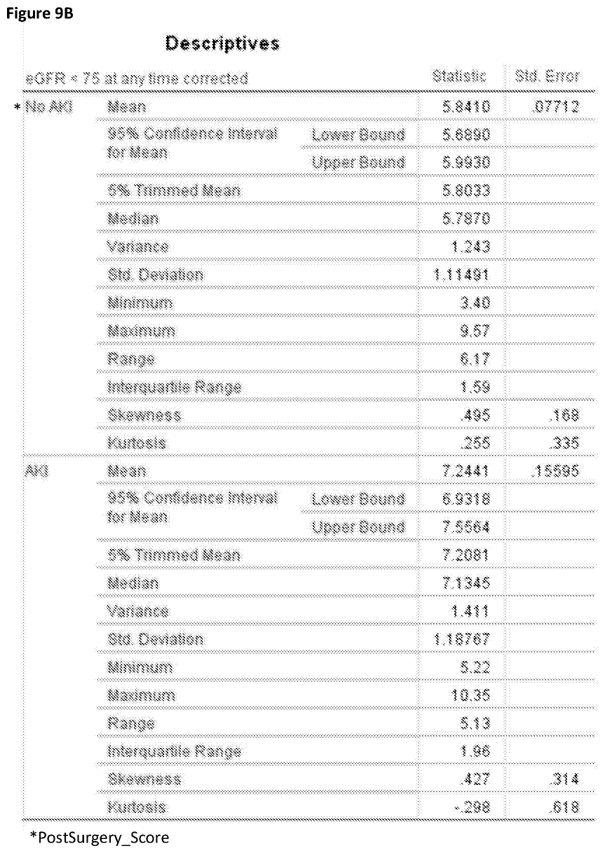

FIG. 9 shows patient scores for a serum post surgery TNFRI, TNFRII and Midkine model;

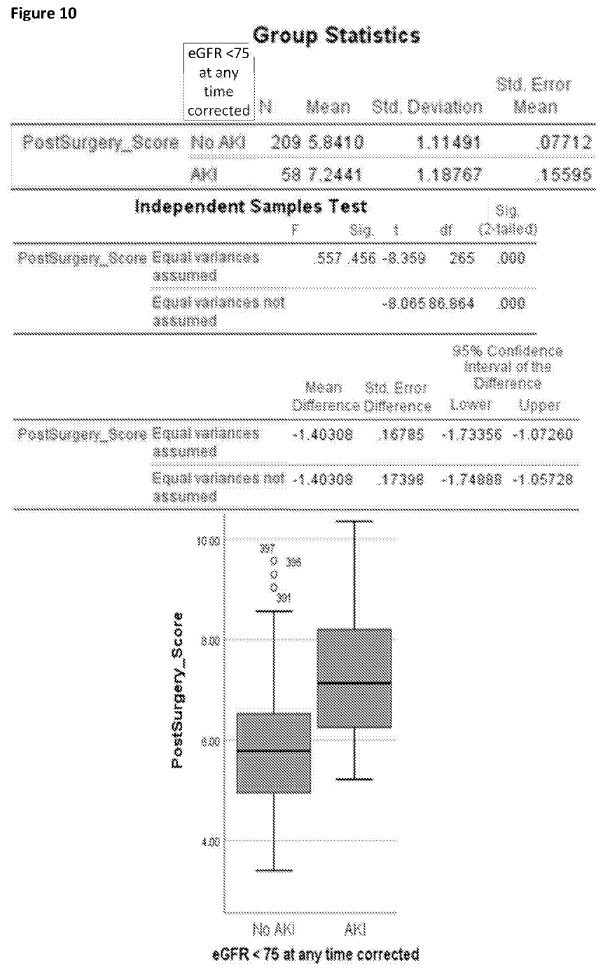

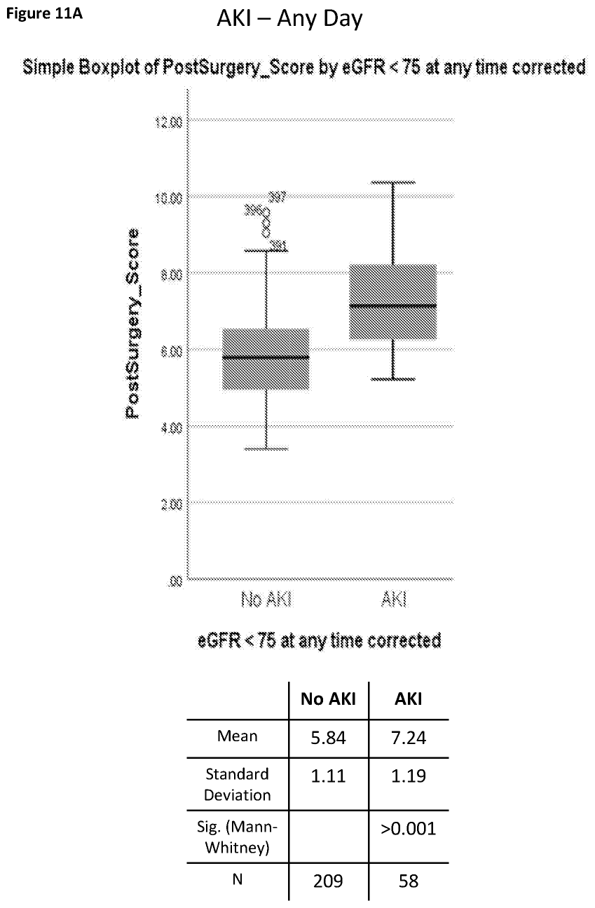

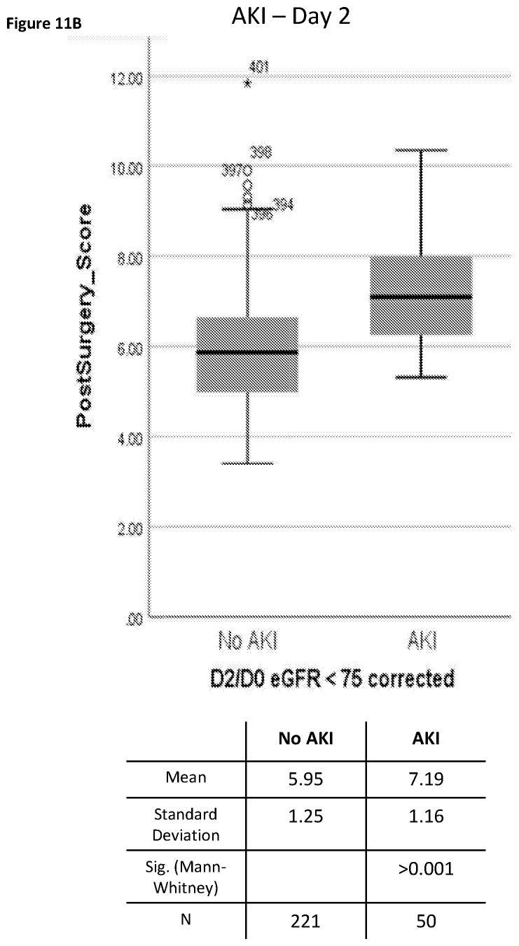

FIG. 10 shows patient scores for a serum post surgery TNFRI, TNFRII and Midkine model;

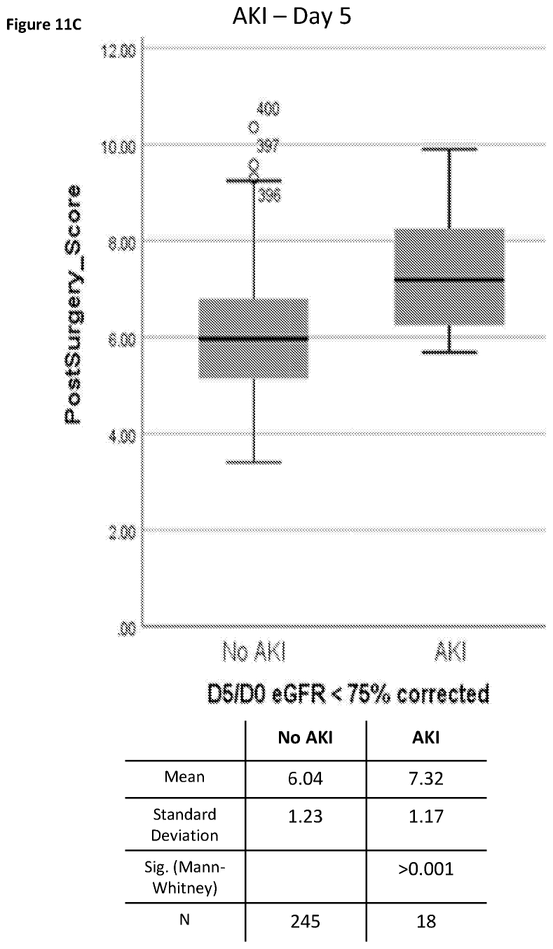

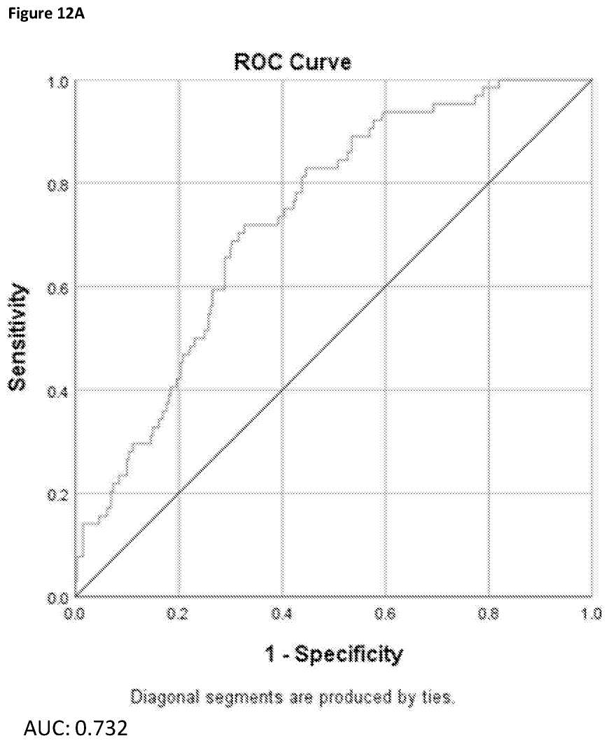

FIG. 11 shows patient scores for a serum post surgery TNFRI, TNFRII and Midkine model;

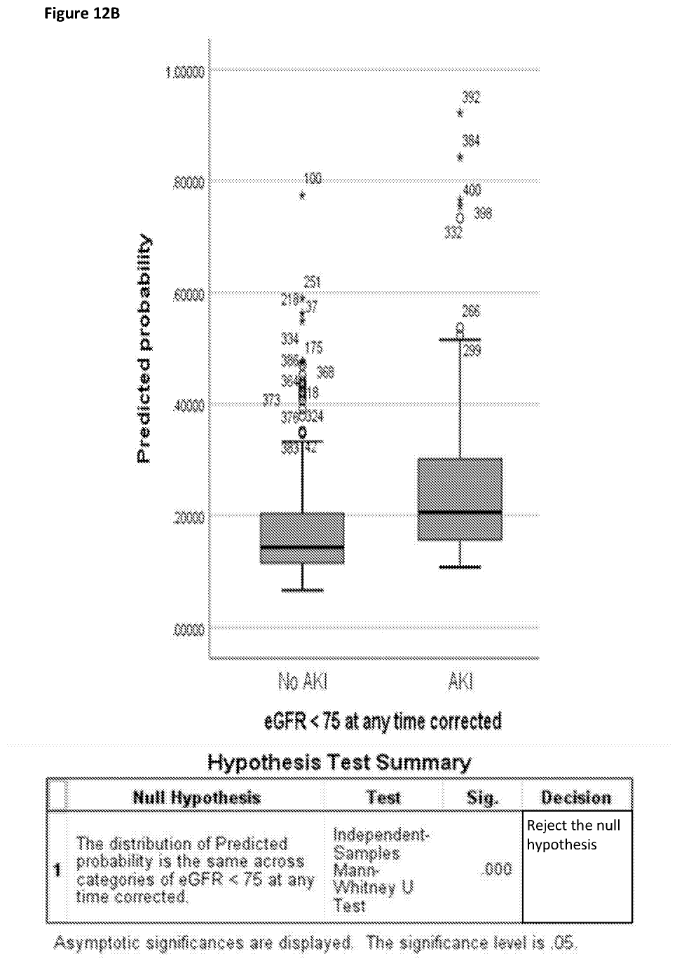

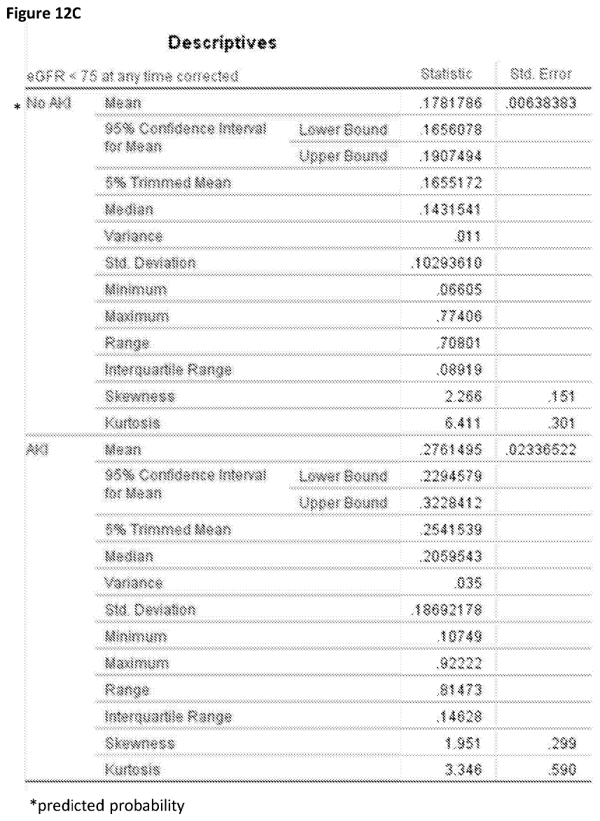

FIG. 12 shows ROC for serum pre-surgery TNFRI and TNFRII;

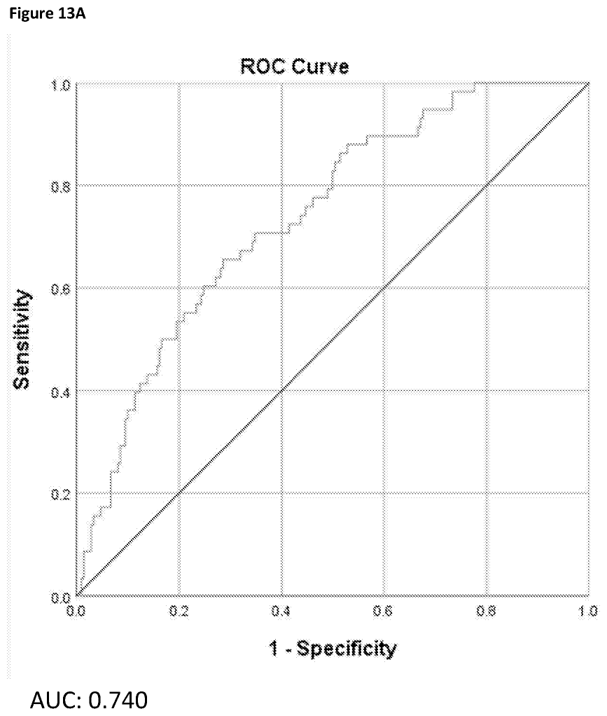

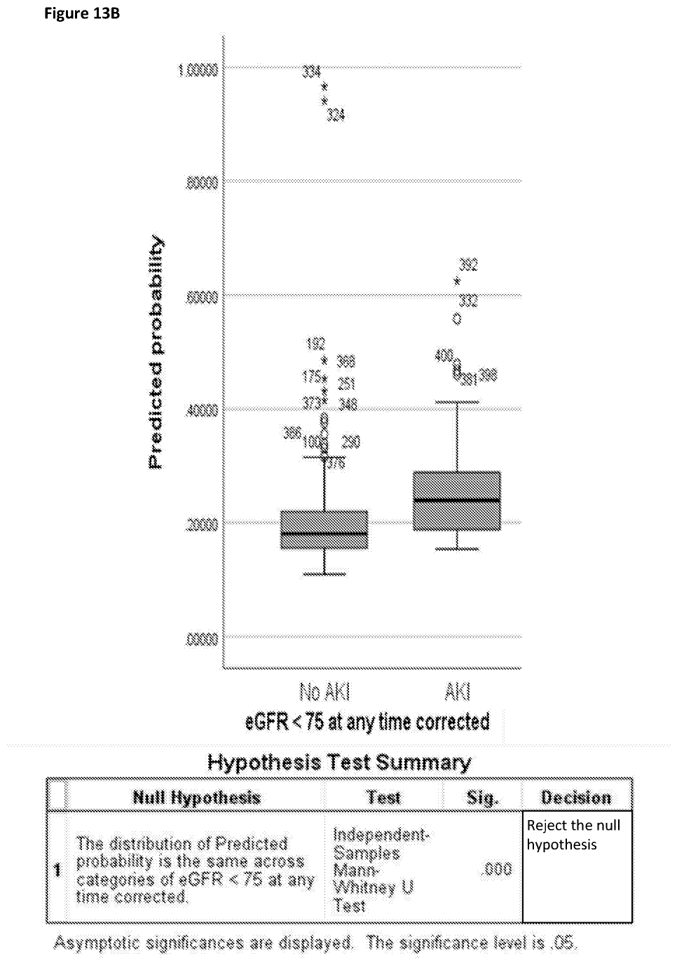

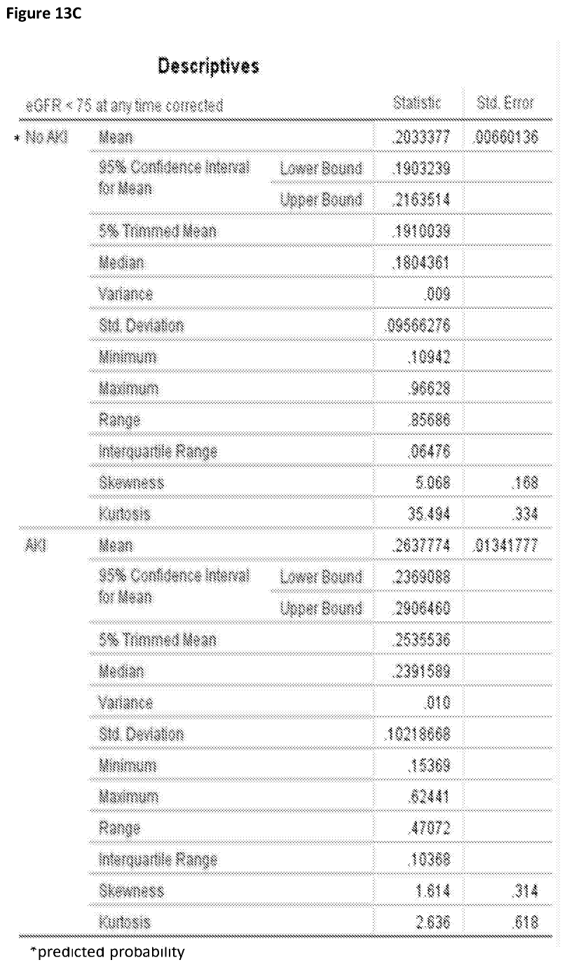

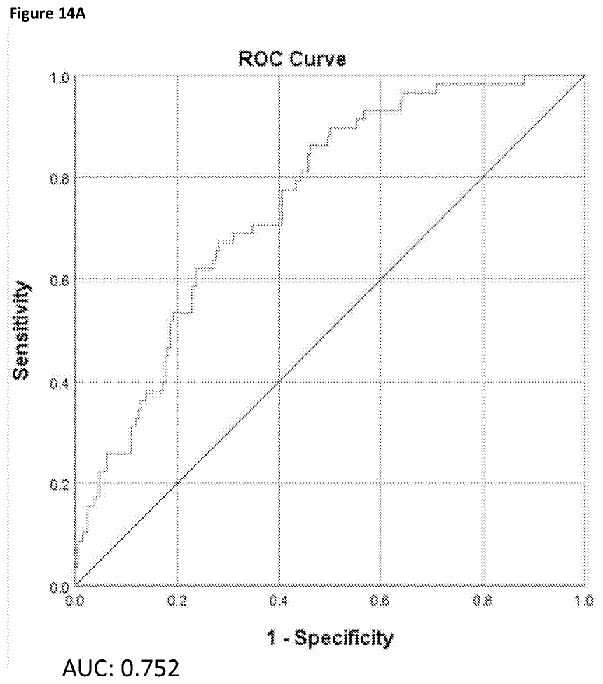

FIG. 13 shows serum pre-surgery TNFRI and Midkine;

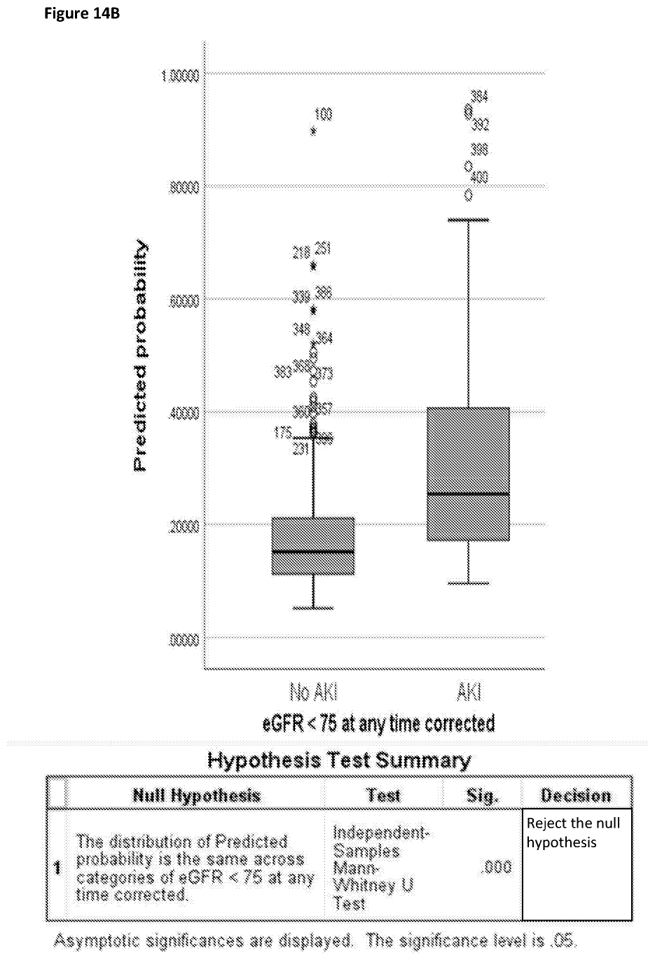

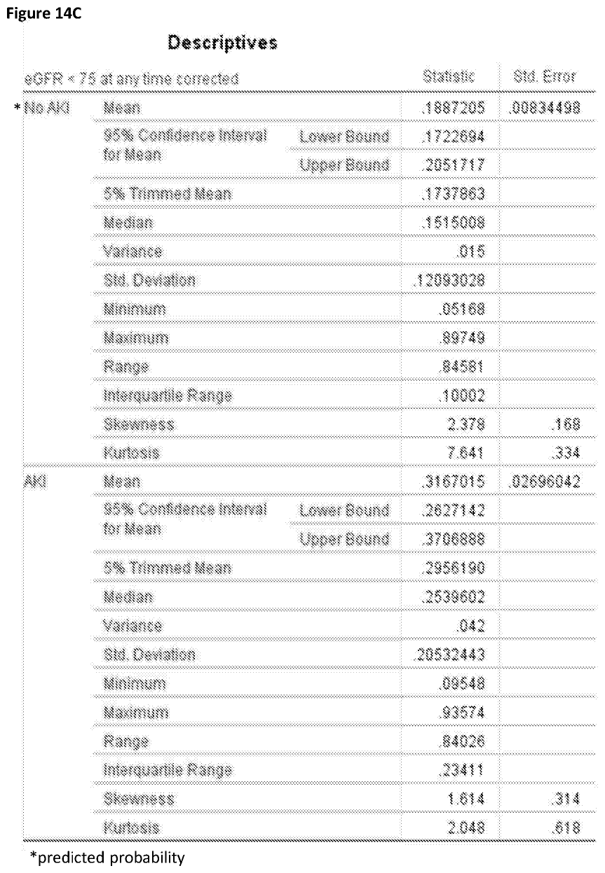

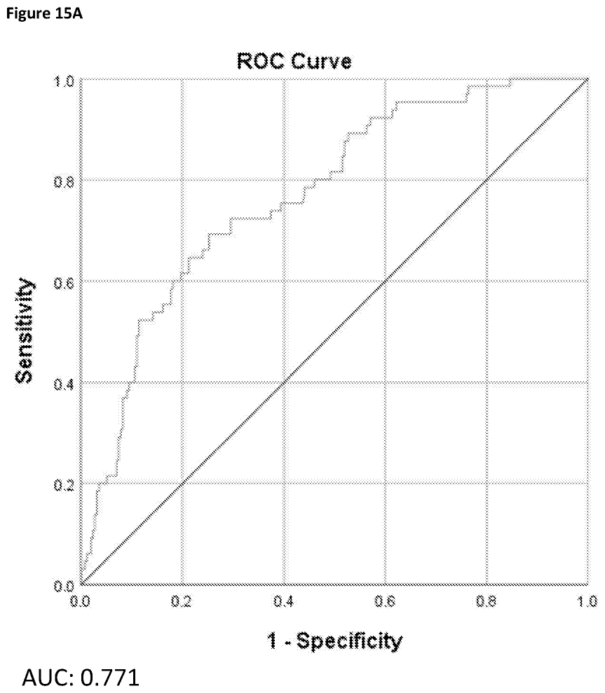

FIG. 14 shows serum pre-surgery TNFRII and Midkine;

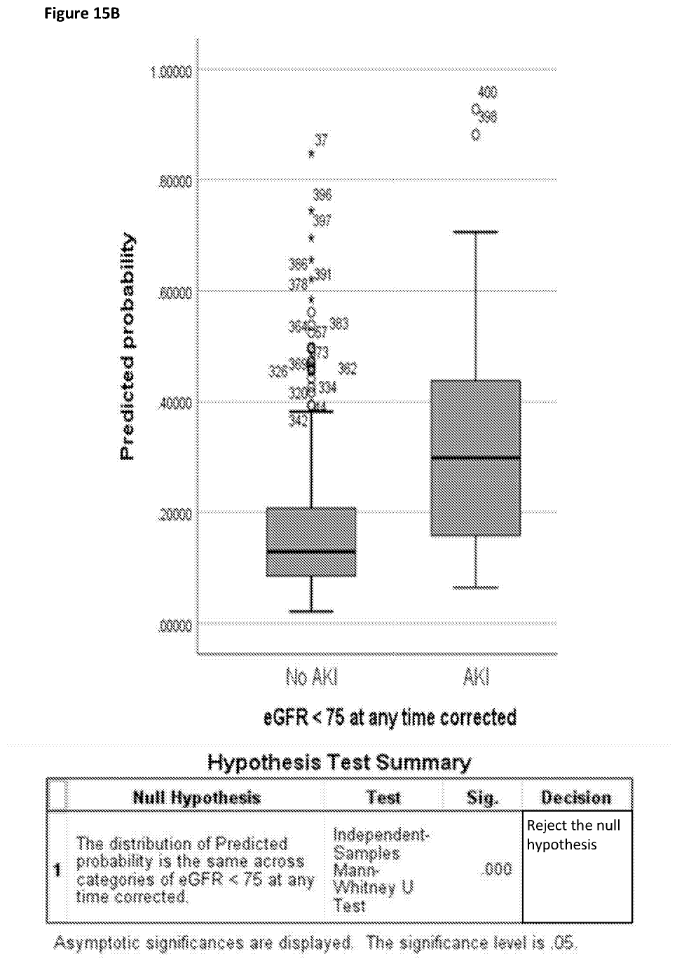

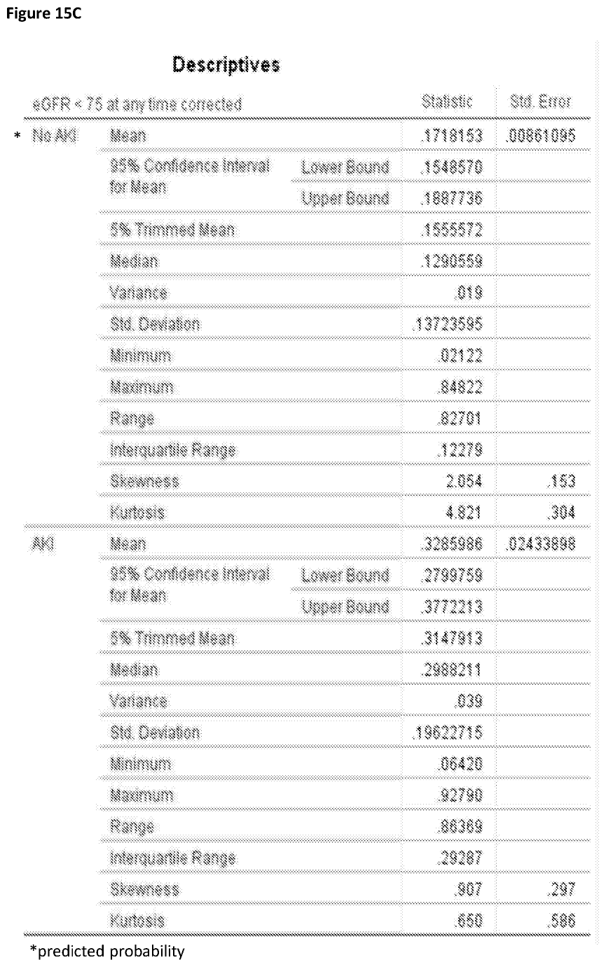

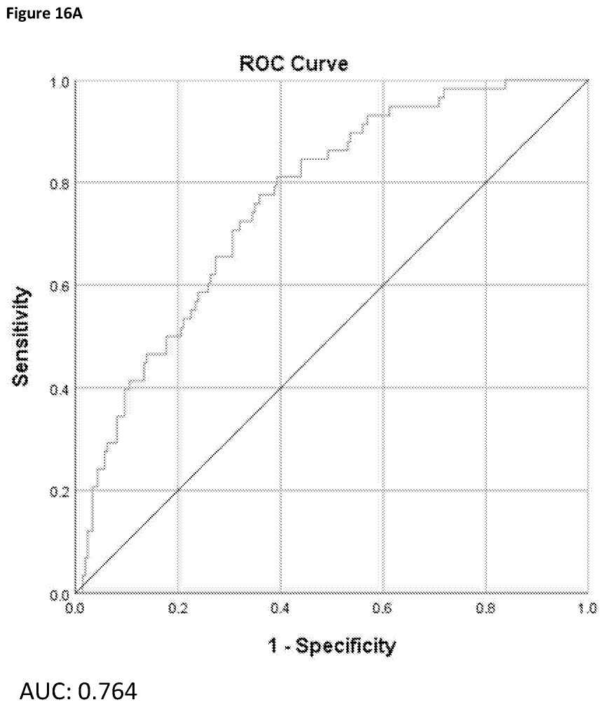

FIG. 15 shows serum post surgery TNFRI and TNFRII;

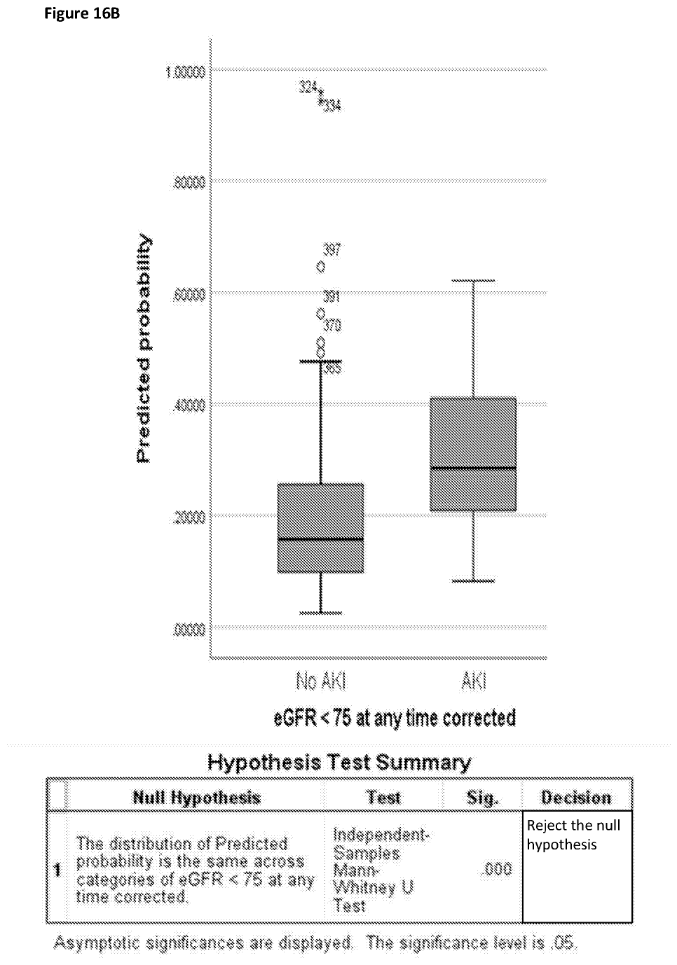

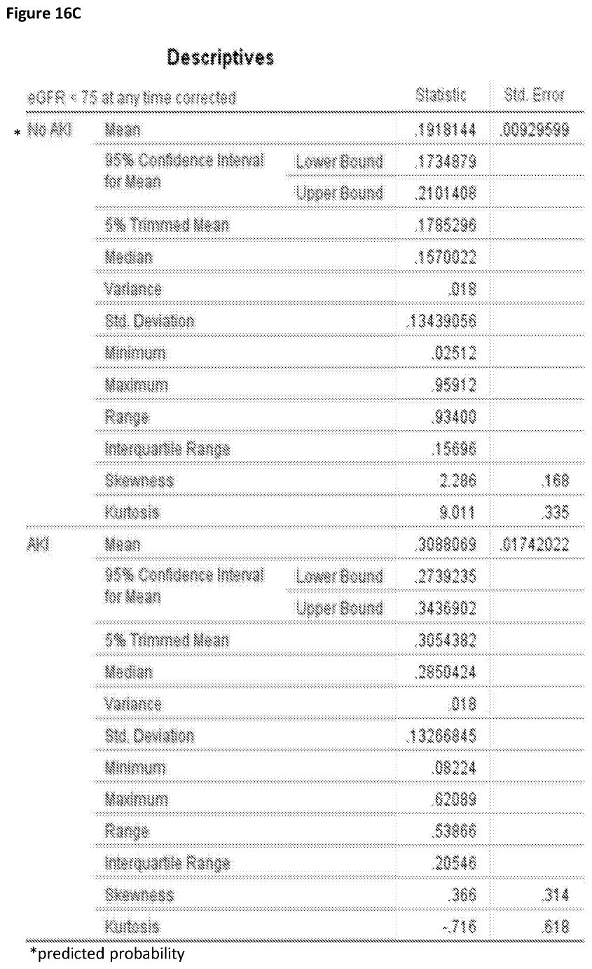

FIG. 16 shows serum post-surgery TNFRI and Midkine;

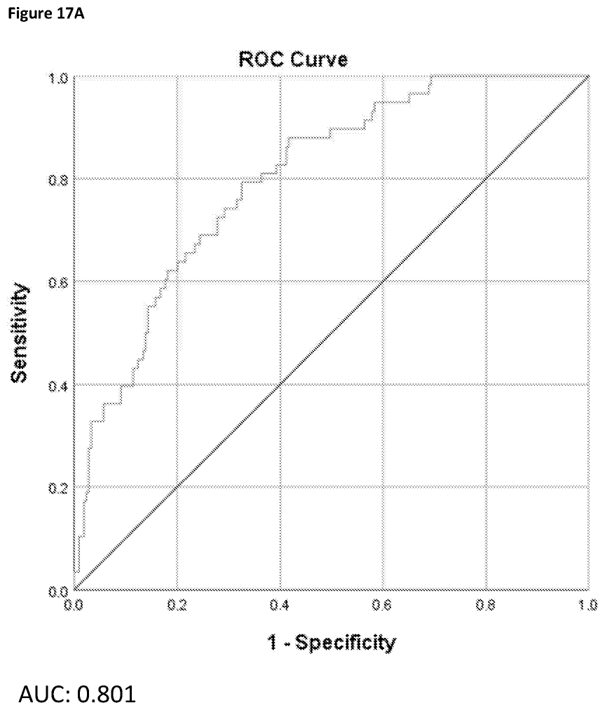

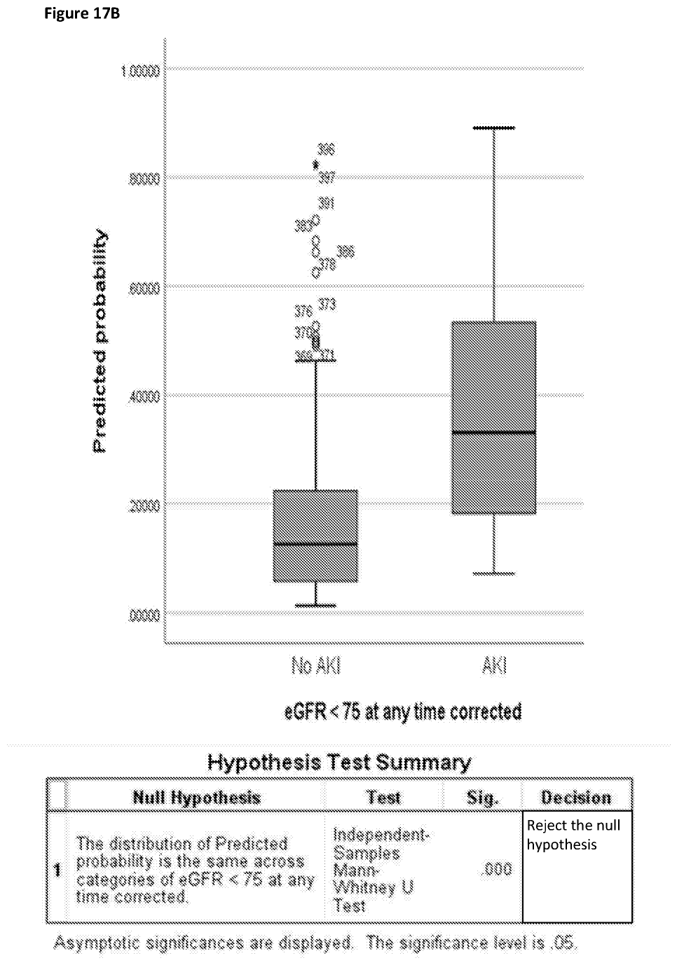

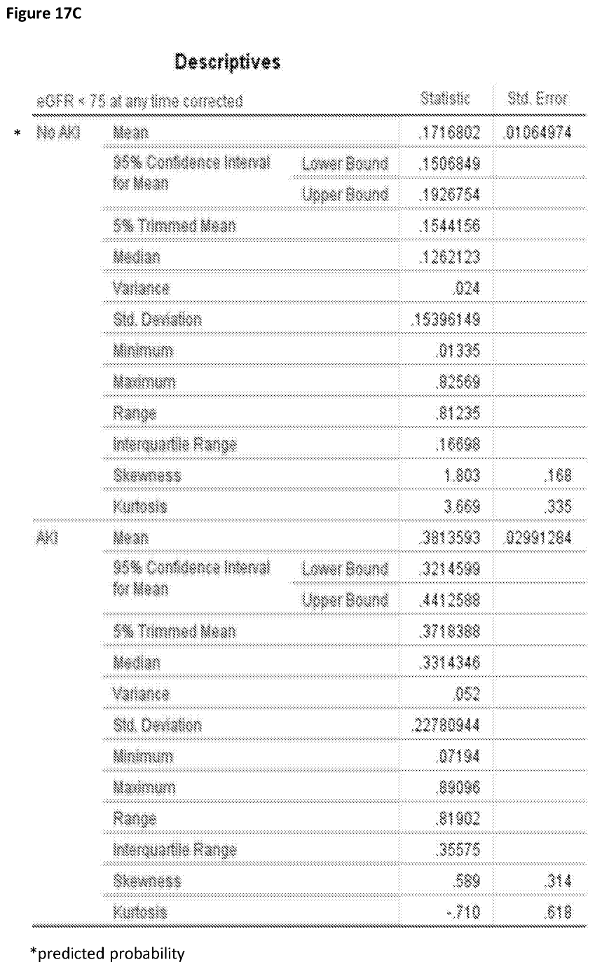

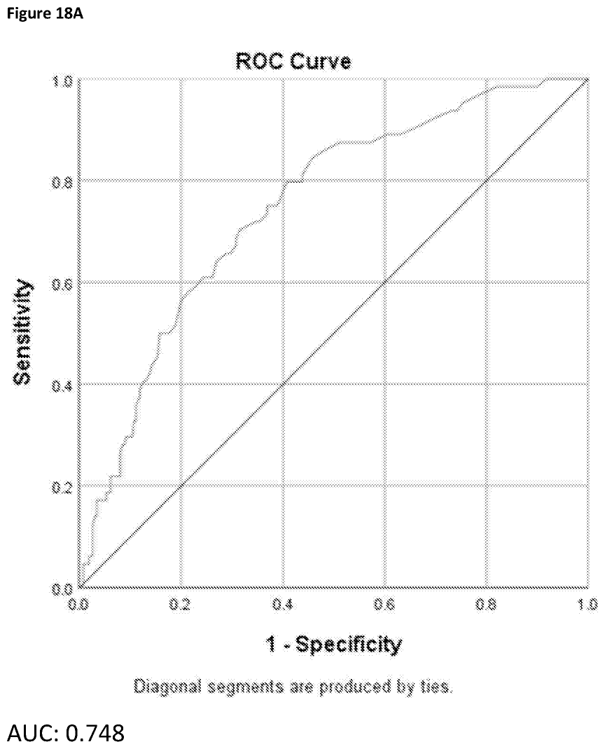

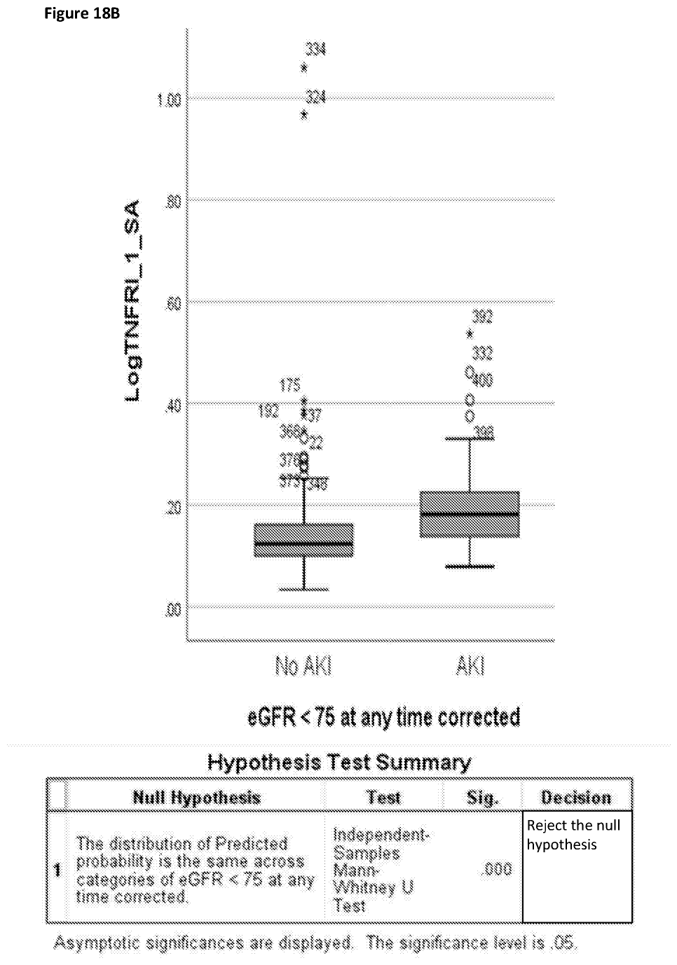

FIG. 17 shows serum post-surgery TNFRII and Midkine;

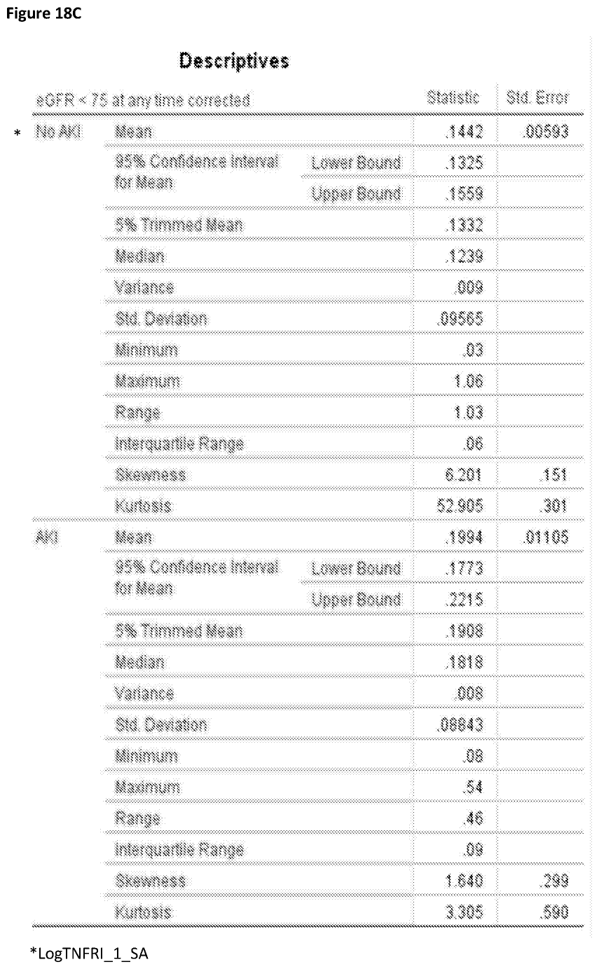

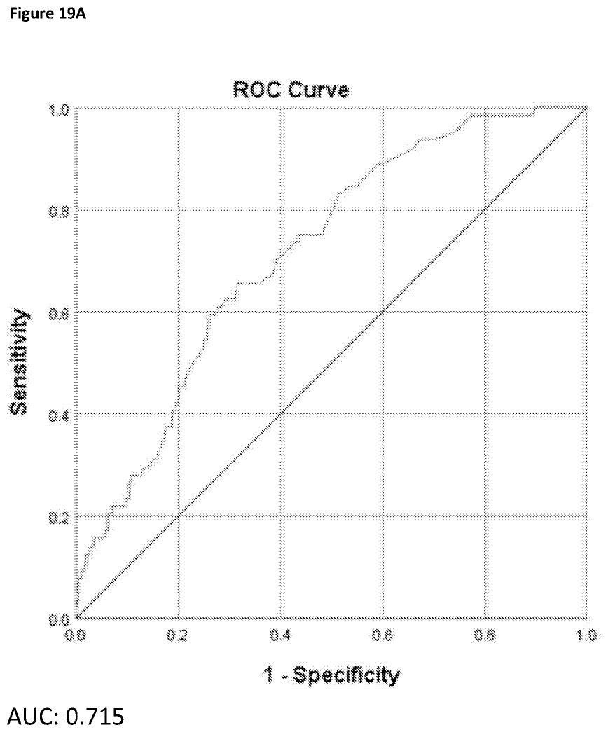

FIG. 18 shows serum pre-surgery TNFRI;

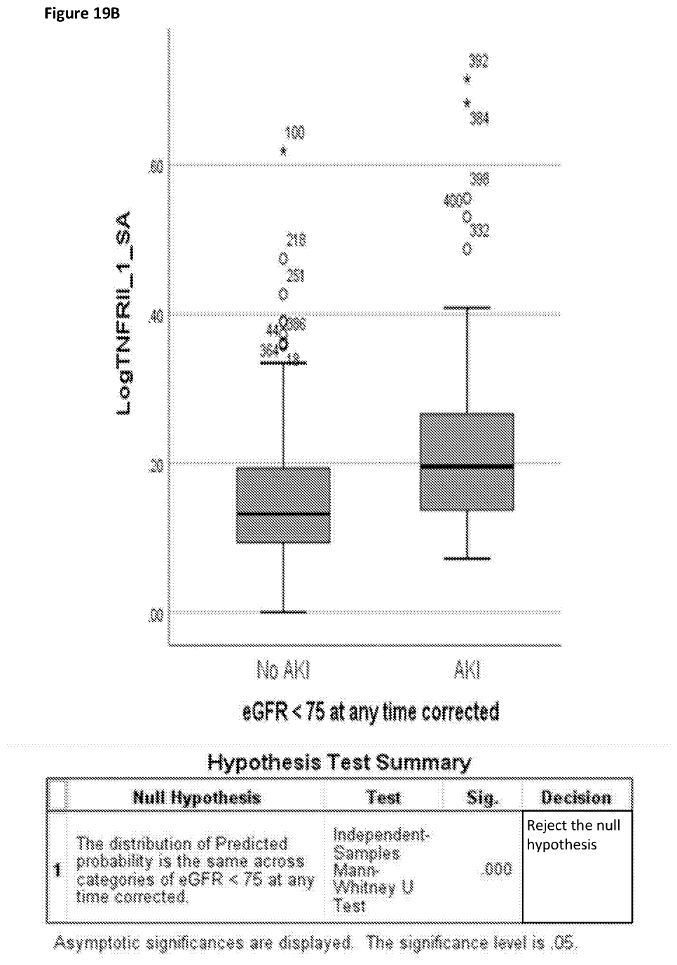

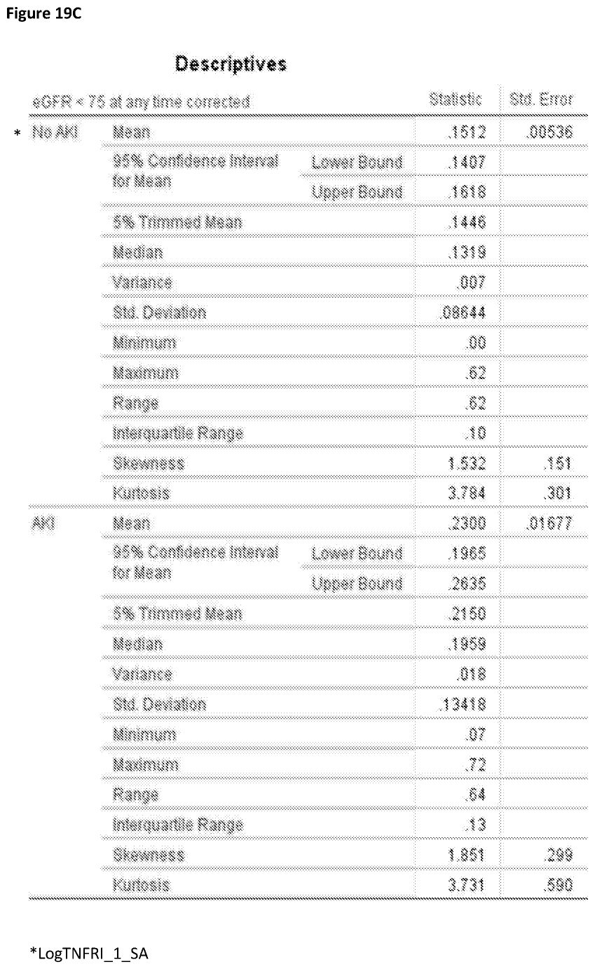

FIG. 19 shows serum pre-surgery TNFRII;

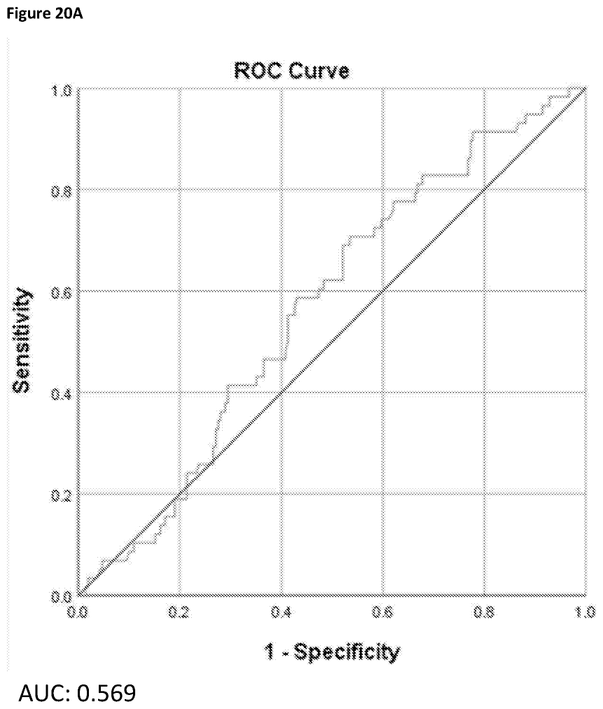

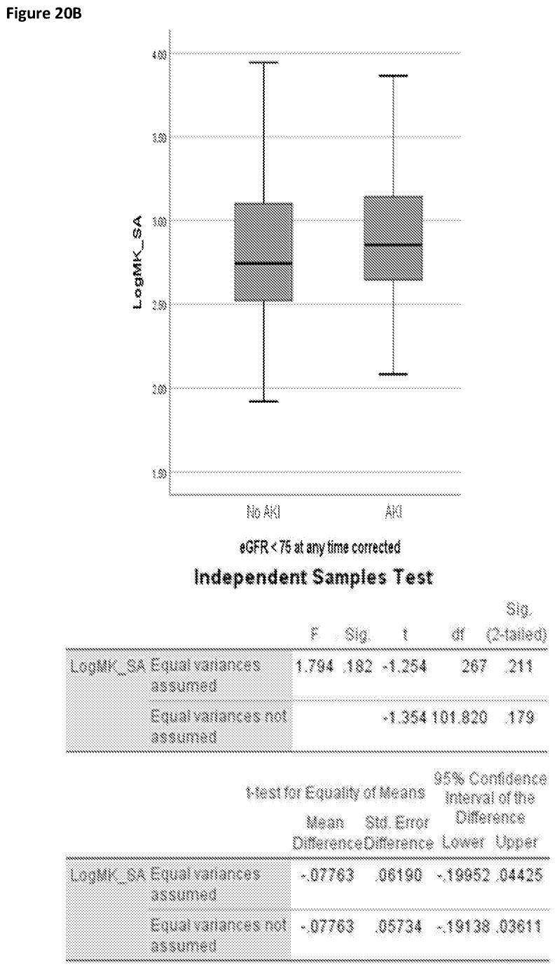

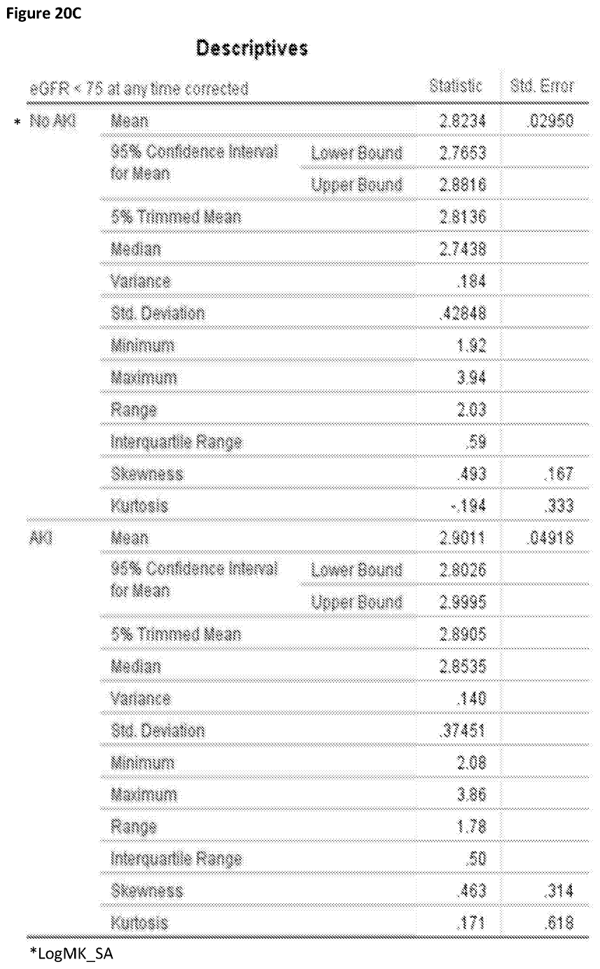

FIG. 20 shows serum pre-surgery Midkine;

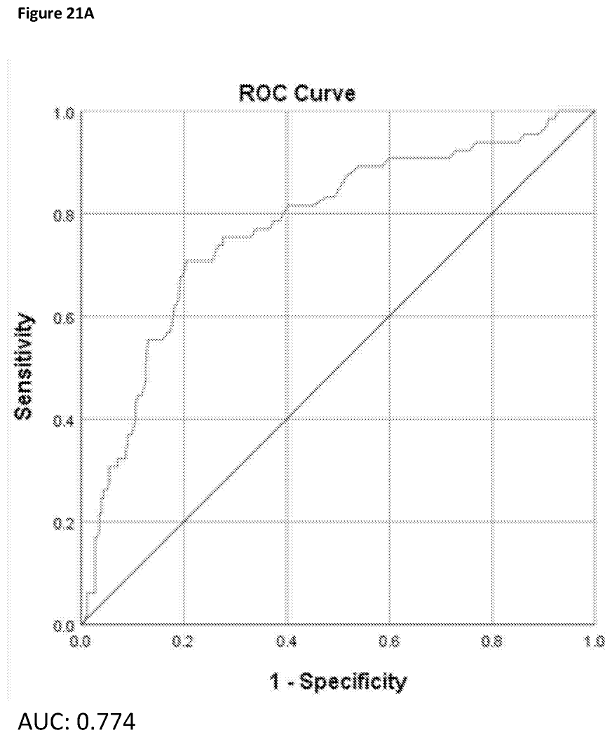

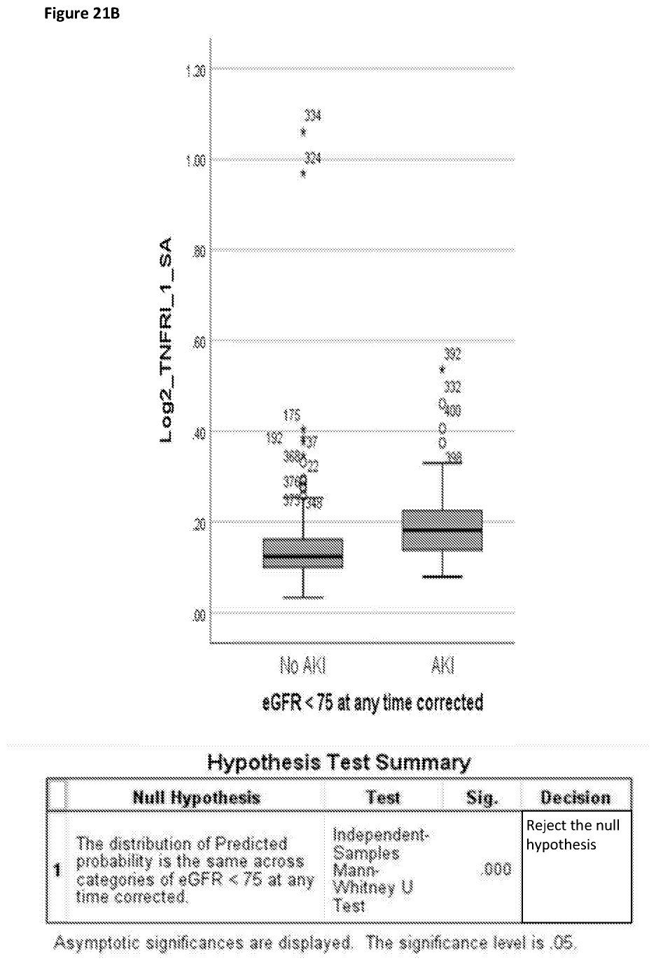

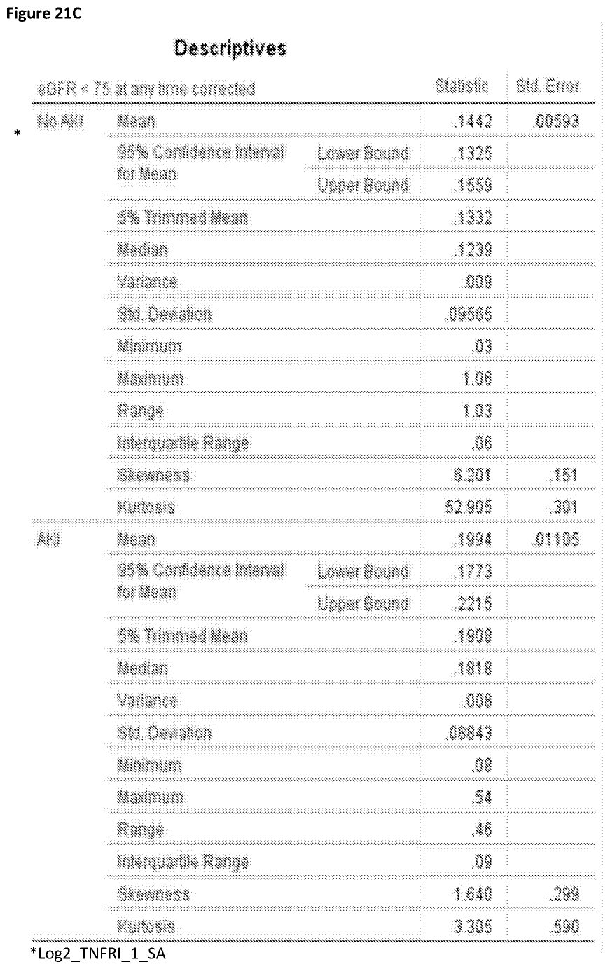

FIG. 21 shows serum post-surgery TNFRI;

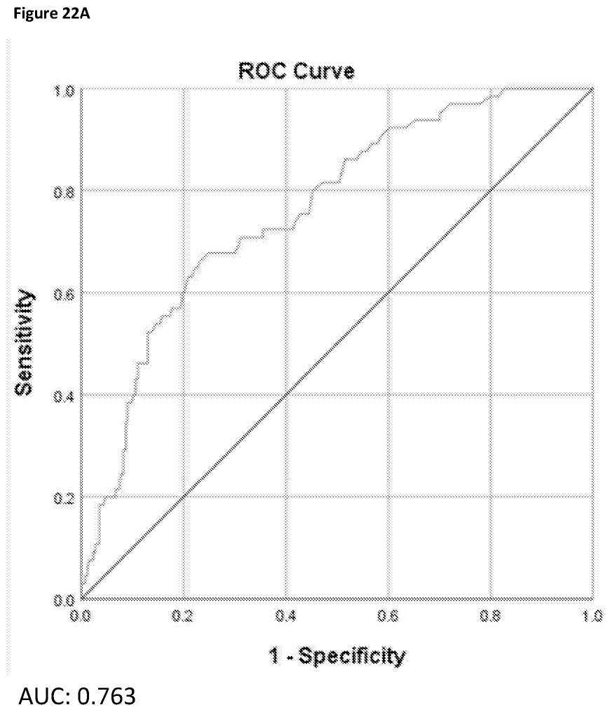

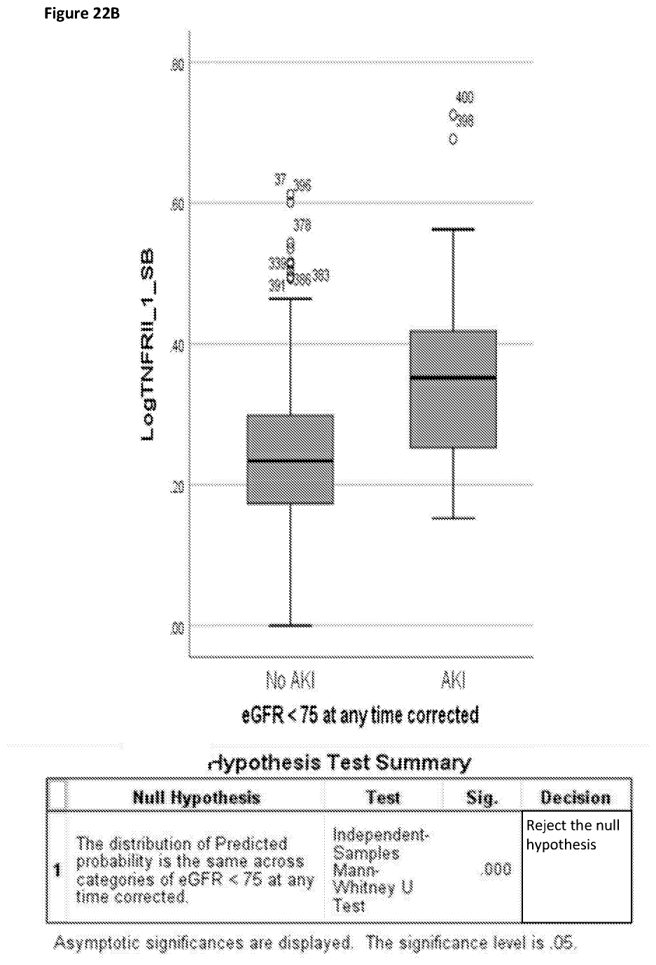

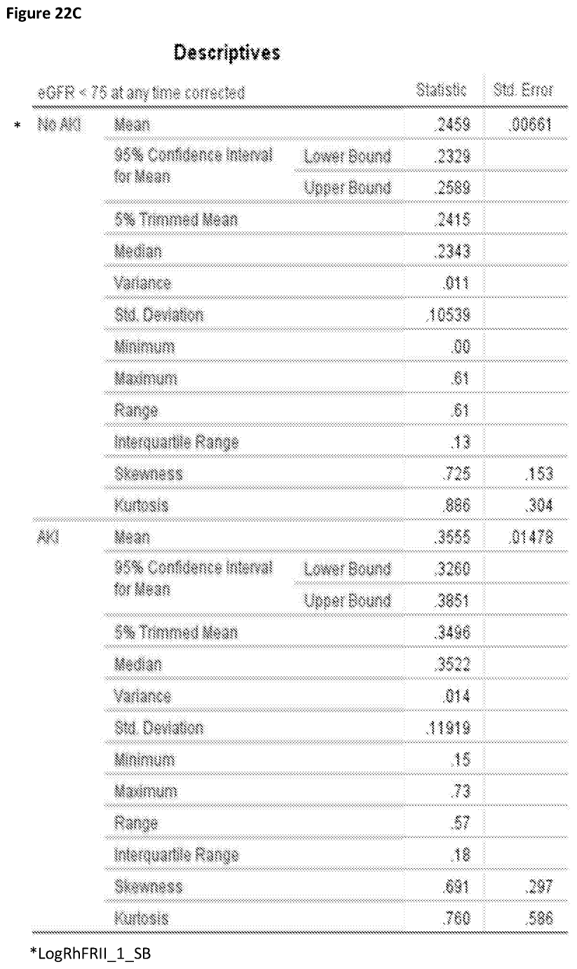

FIG. 22 shows serum post-surgery TNFRII;

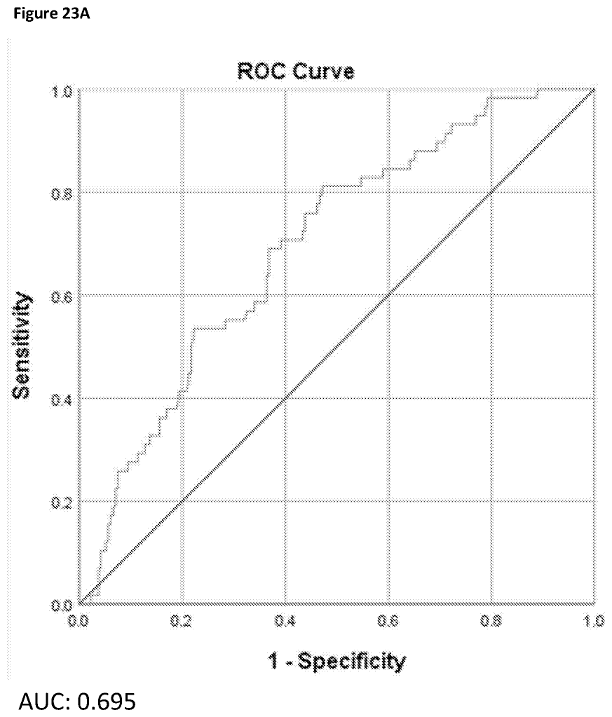

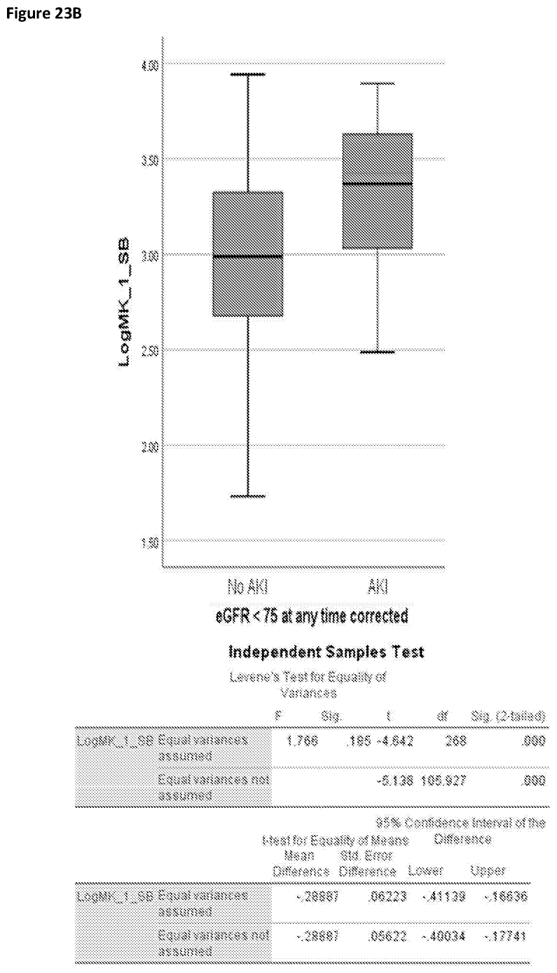

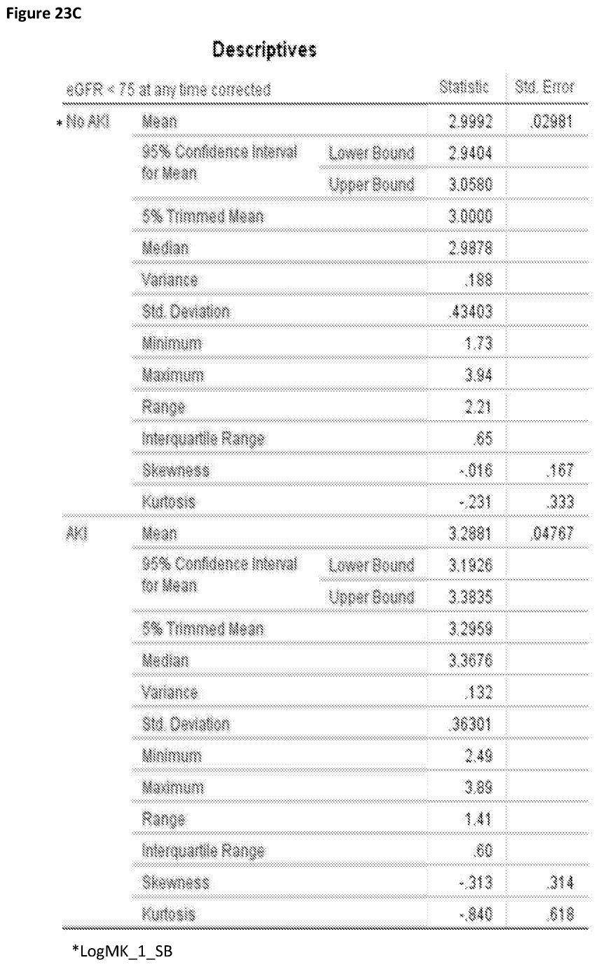

FIG. 23 shows serum post-surgery Midkine;

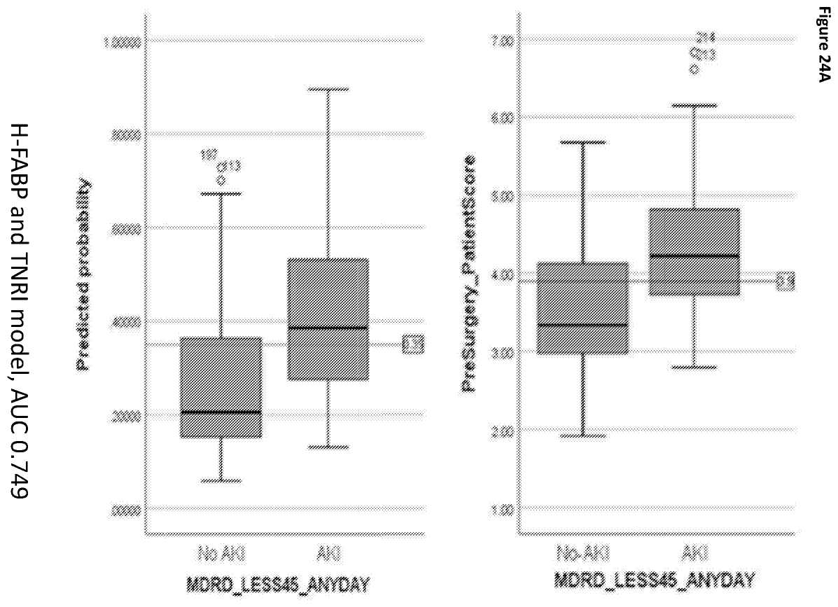

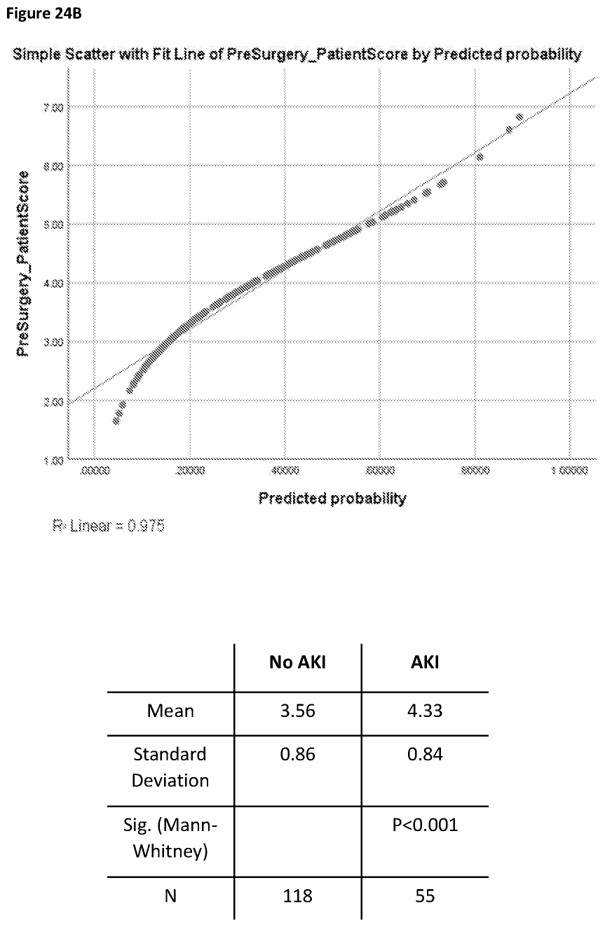

FIGS. 24 A and B show serum pre-fracture repair surgery;

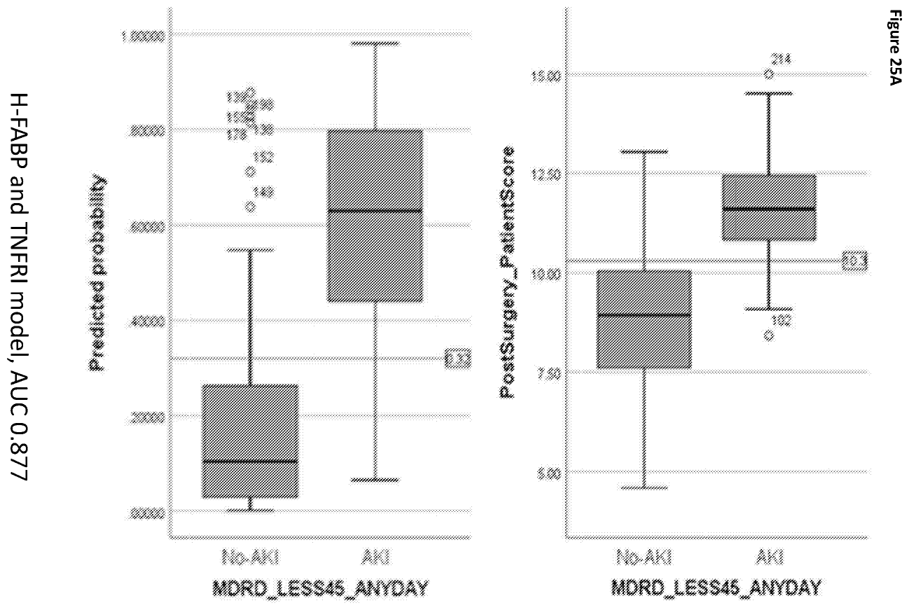

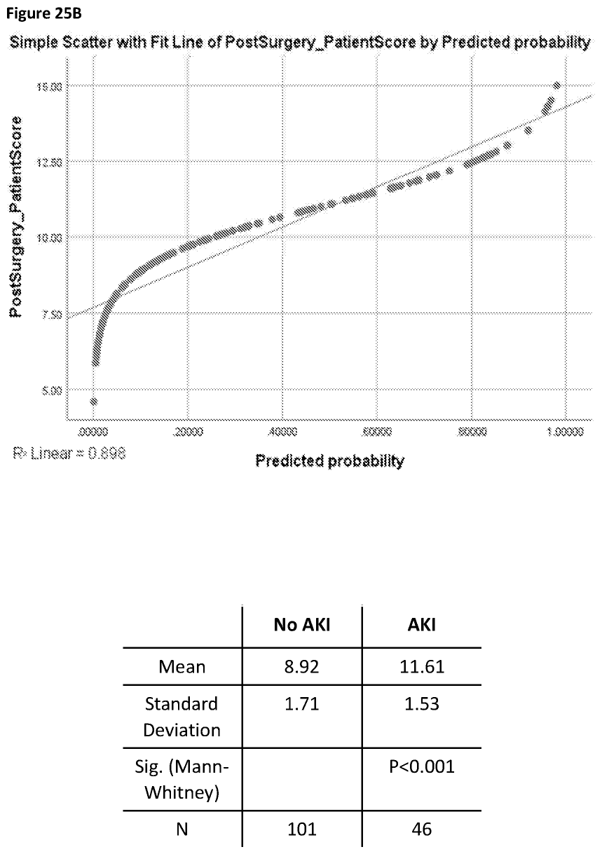

FIGS. 25 A and B show serum post fracture repair surgery;

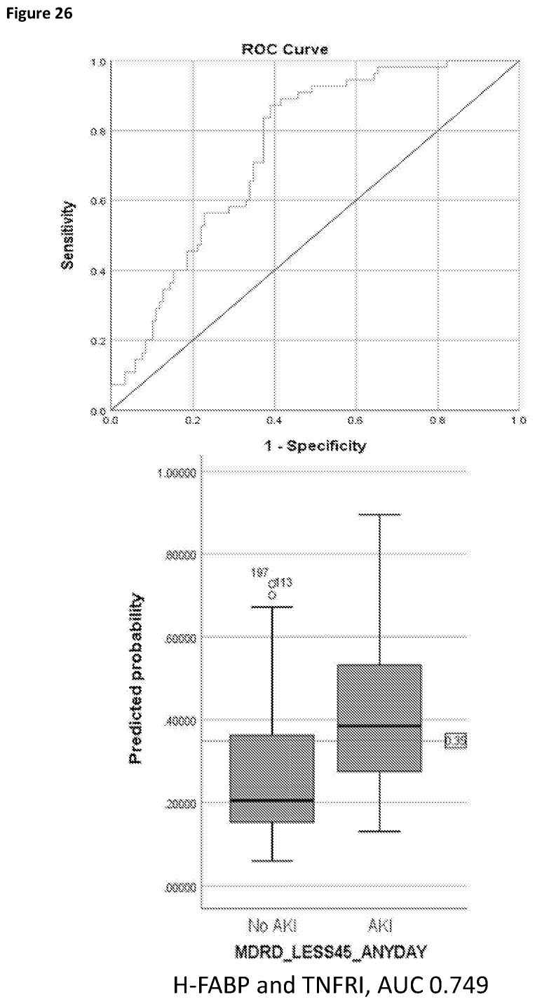

FIG. 26 illustrates Serum pre-surgery predictive model H-FABP and TNFRI;

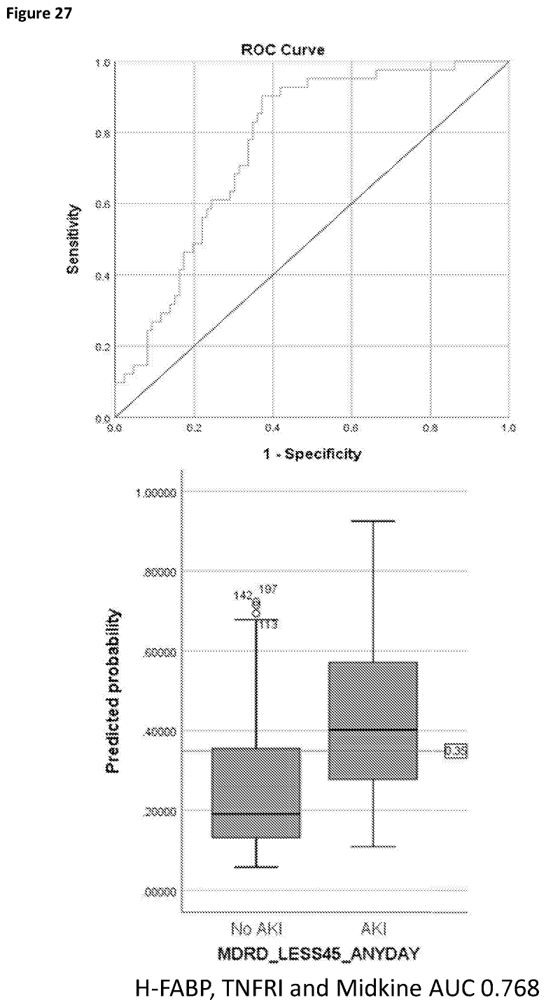

FIG. 27 illustrates Serum pre-surgery predictive model H-FABP, TNFRI and Midkine;

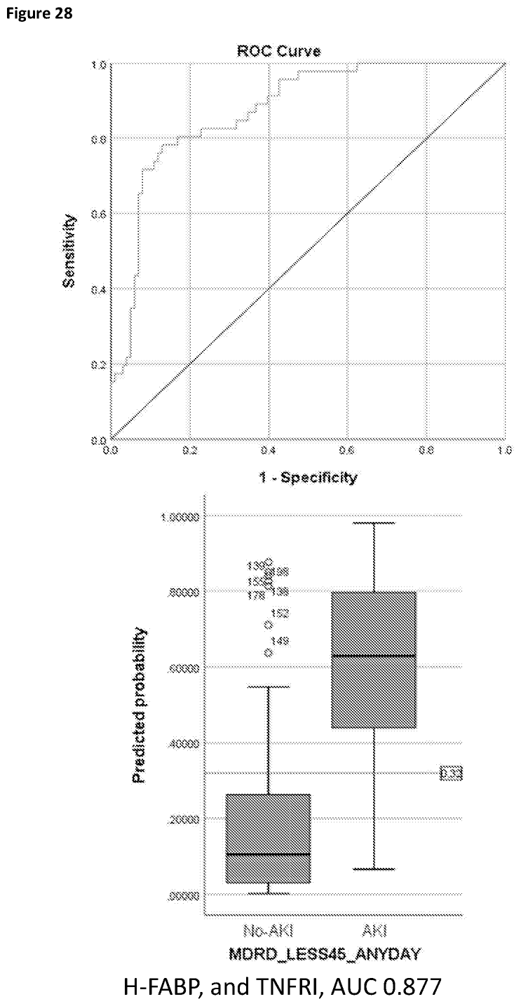

FIG. 28 illustrates Serum post-surgery model H-FABP and TNFRI;

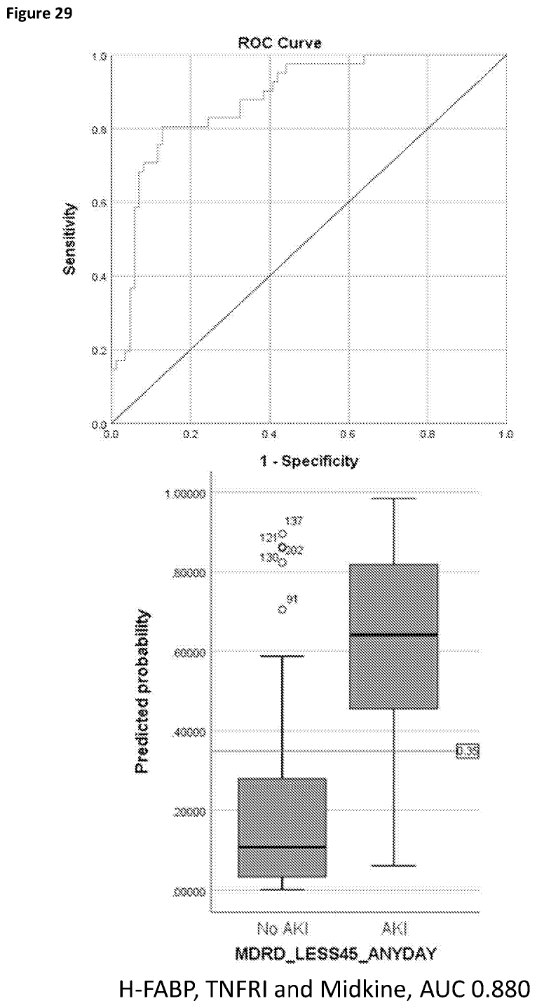

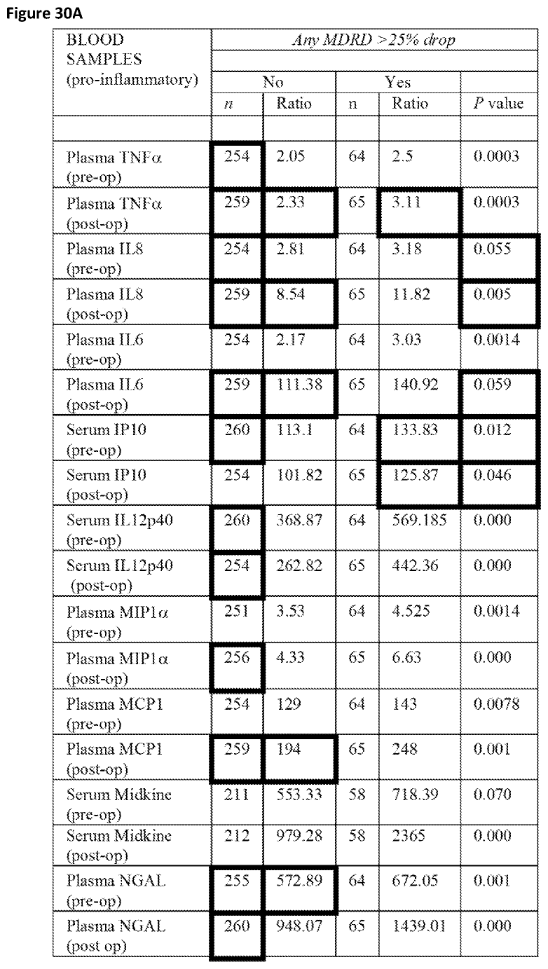

FIG. 29 illustrates Serum post-surgery H-FABP, TNFRI and Midkine biomarker model;

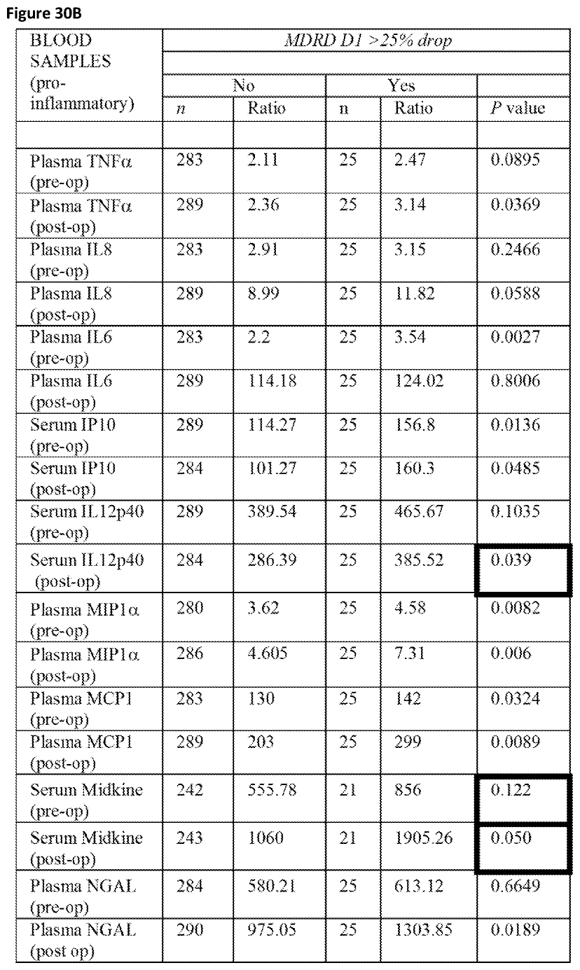

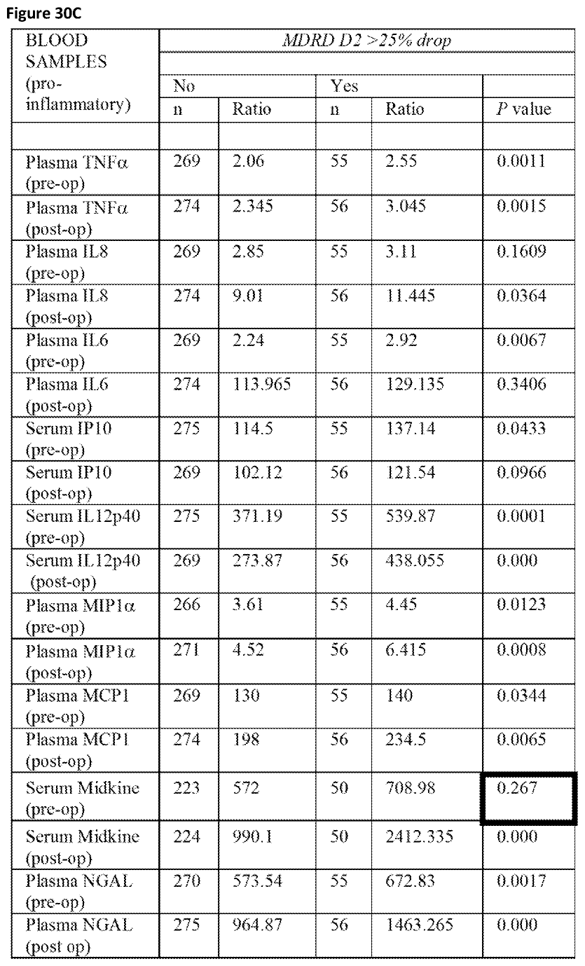

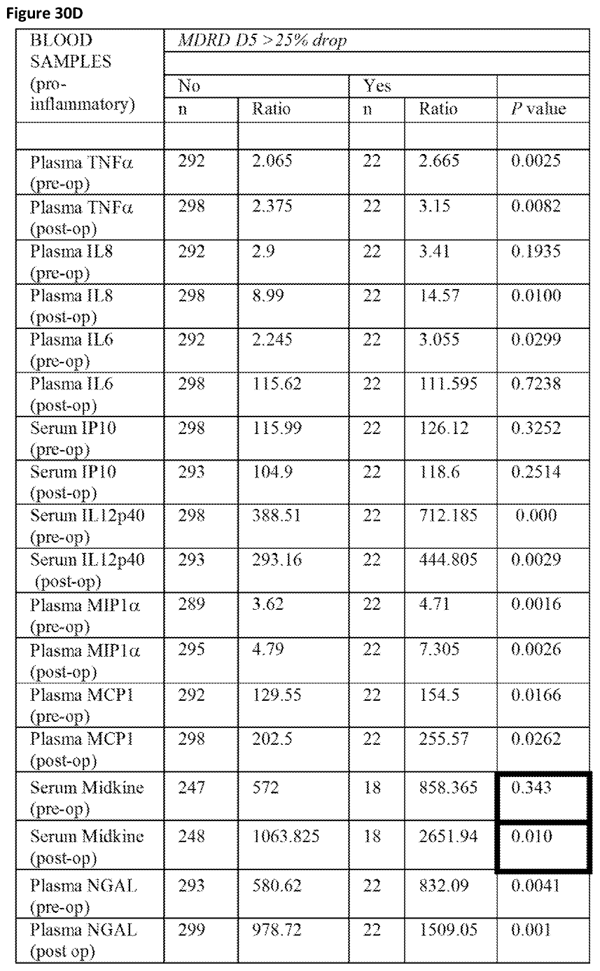

FIG. 30 illustrates biomarker results;

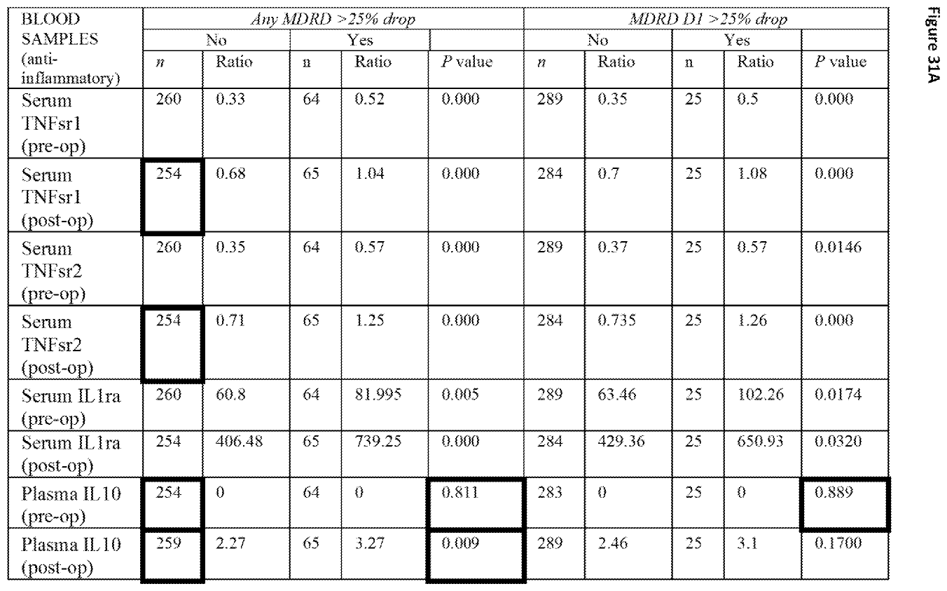

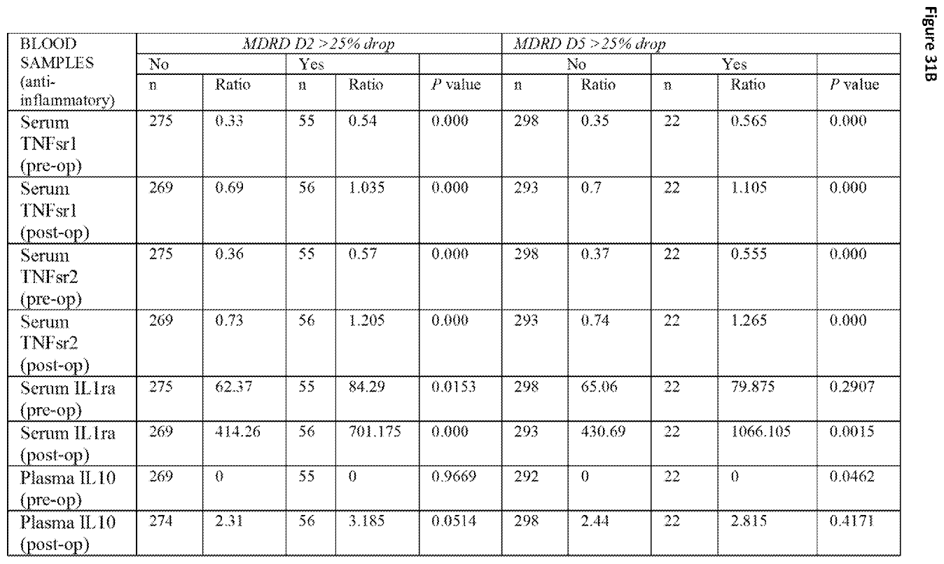

FIG. 31 illustrates biomarker results;

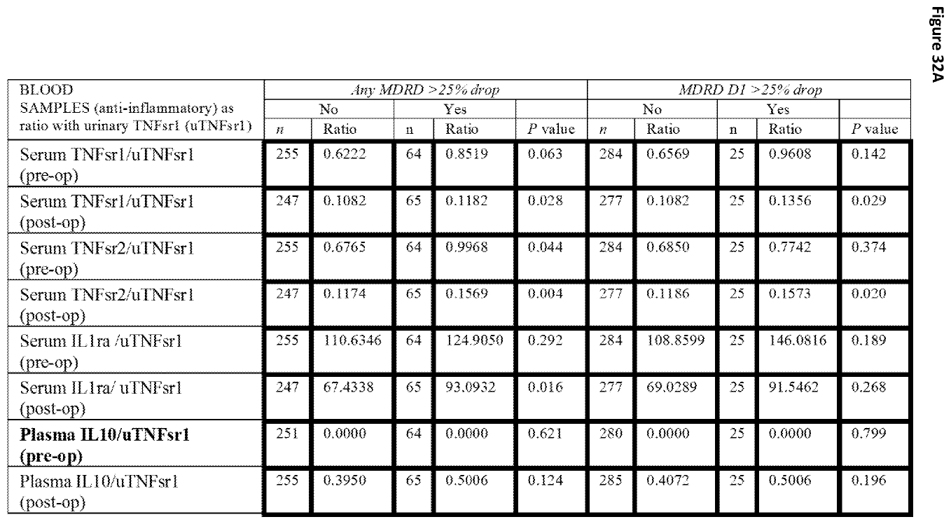

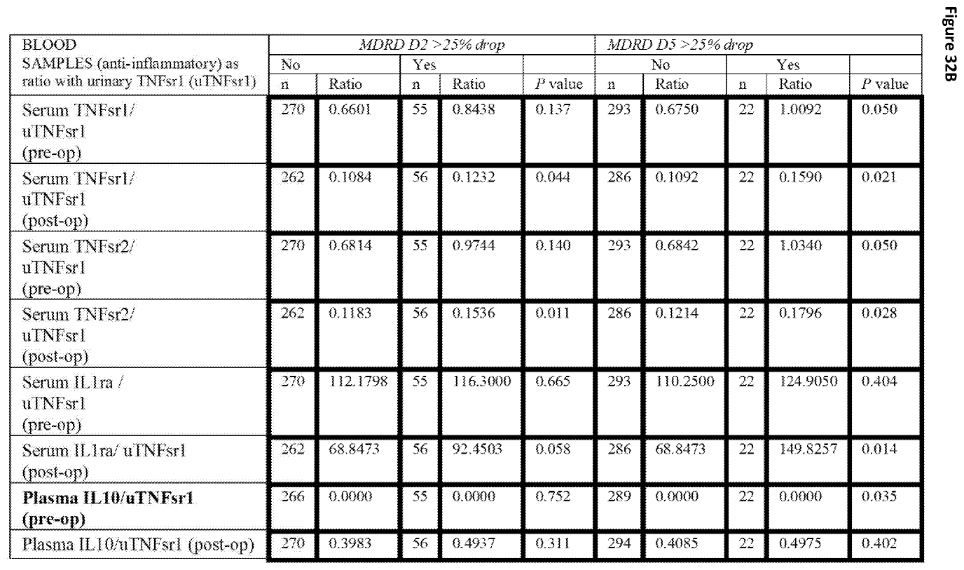

FIG. 32 illustrates biomarker results;

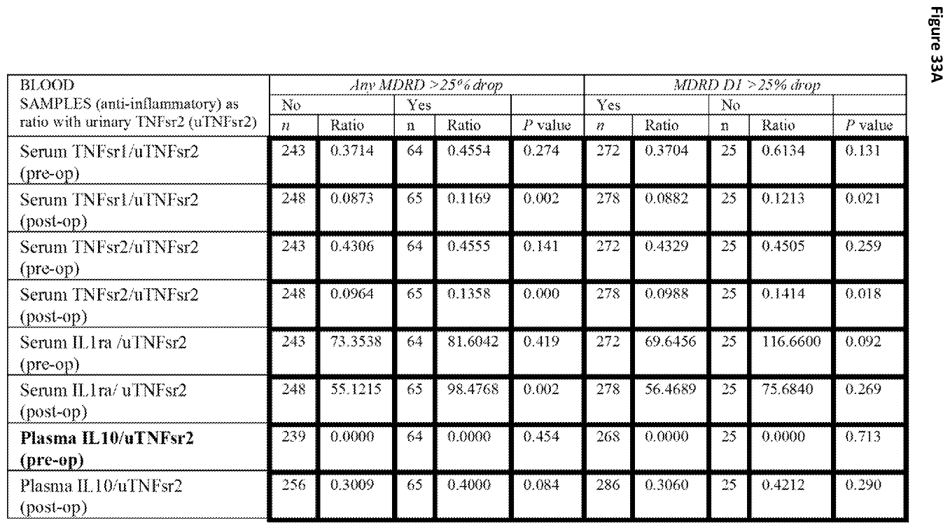

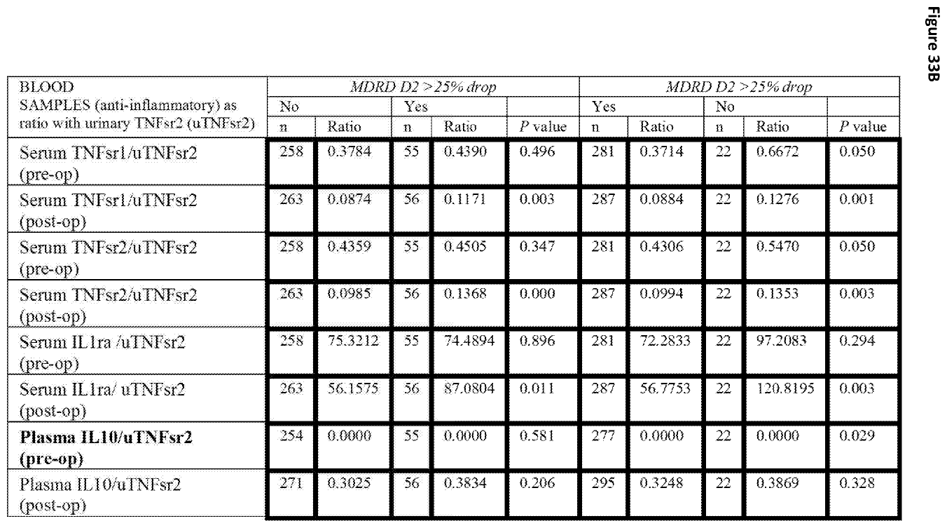

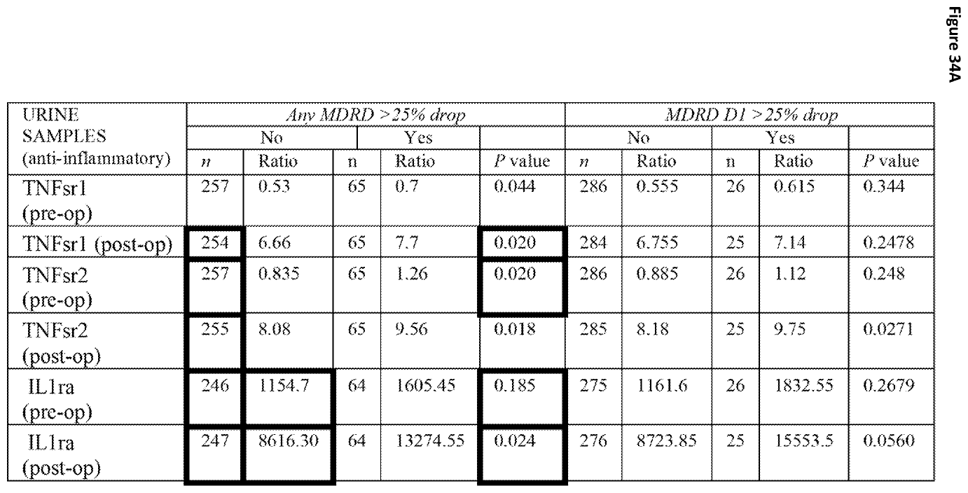

FIG. 33 illustrates biomarker results;

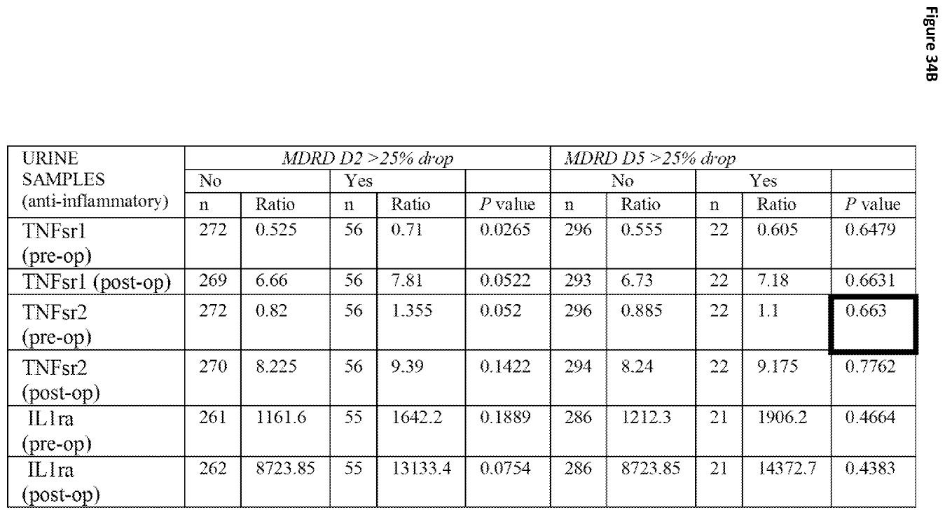

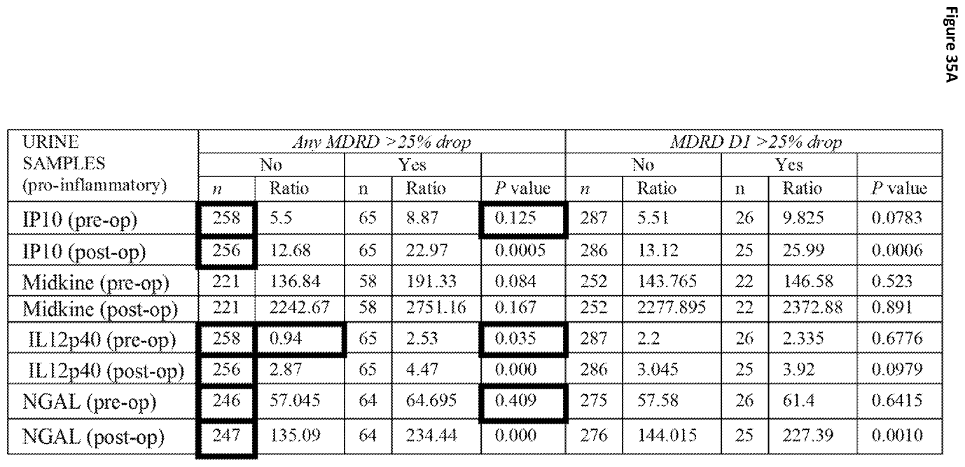

FIG. 34 illustrates biomarker results;

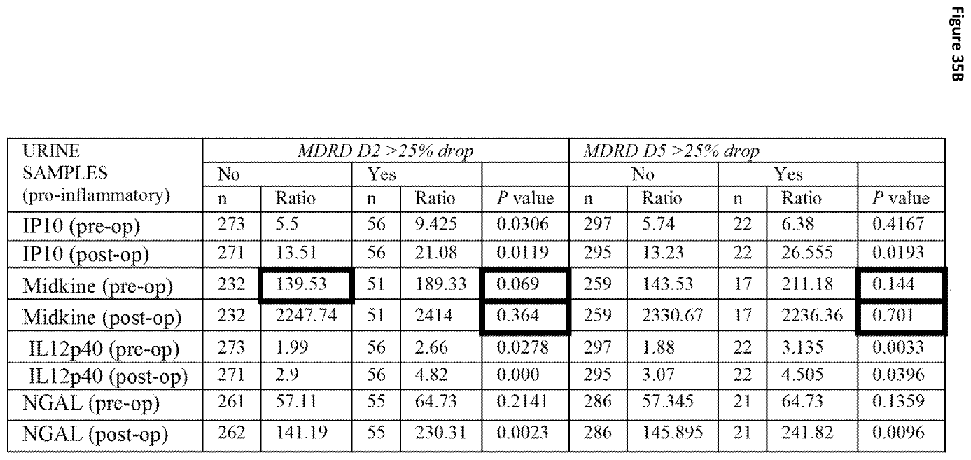

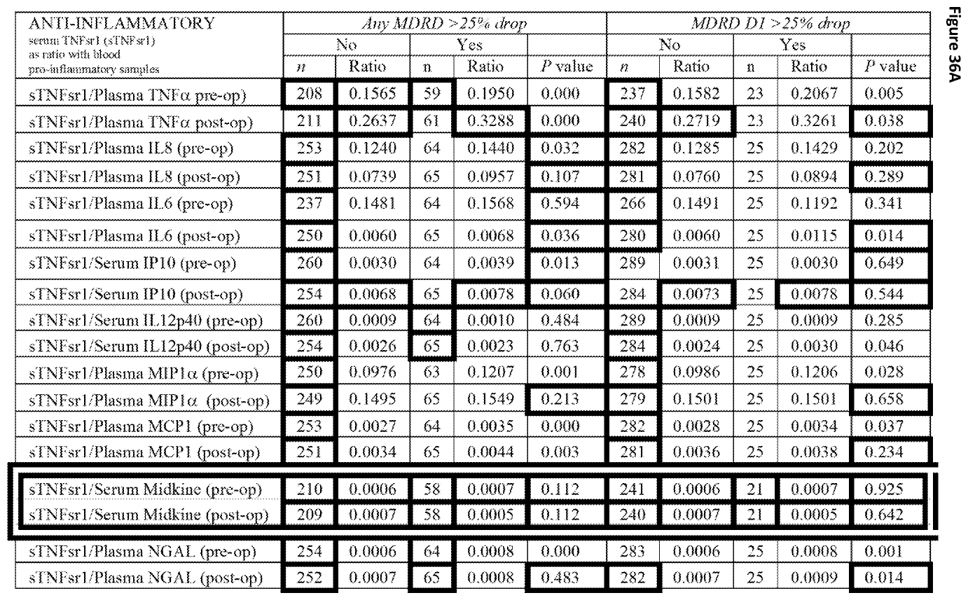

FIG. 35 illustrates biomarker results;

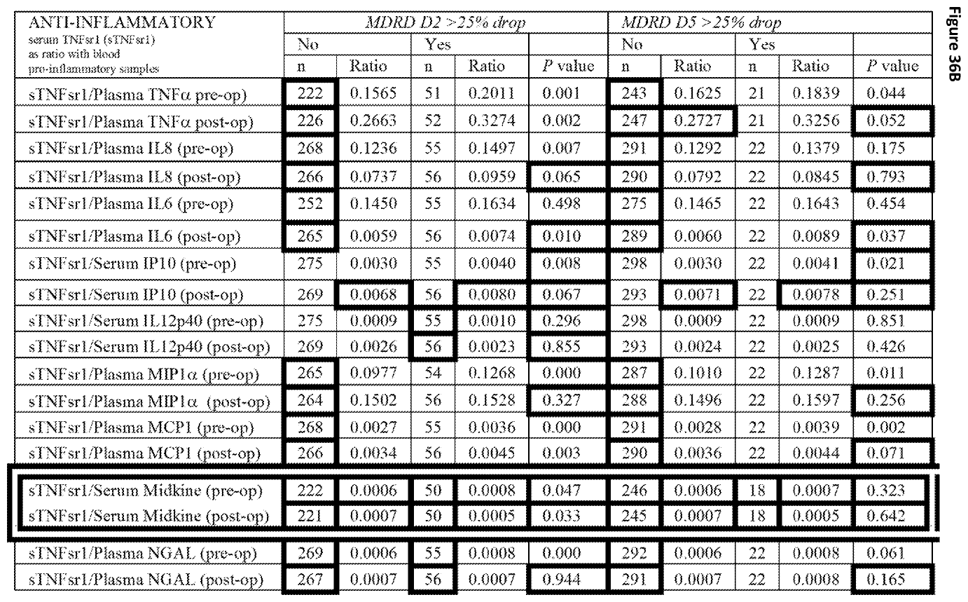

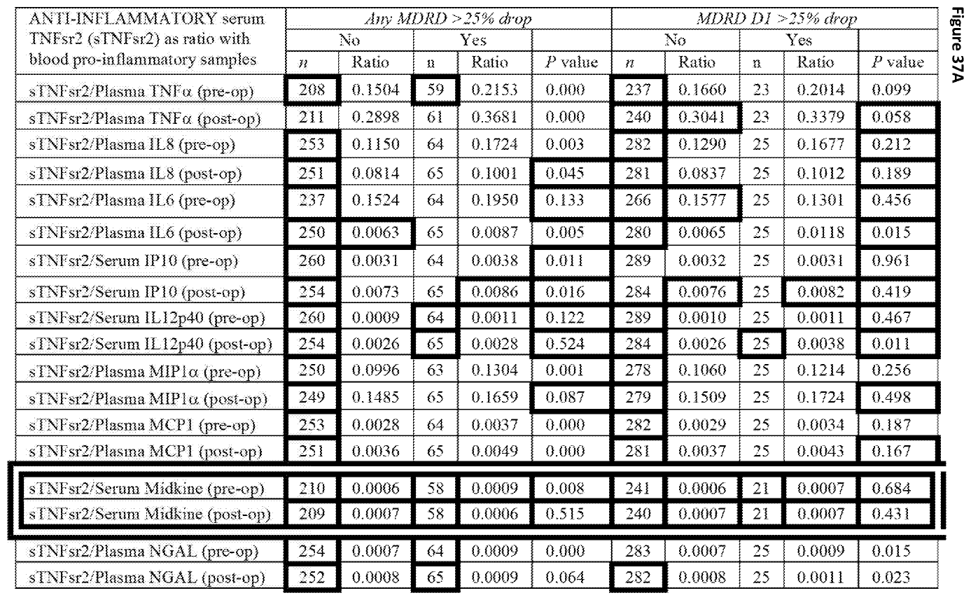

FIG. 36 illustrates biomarker results;

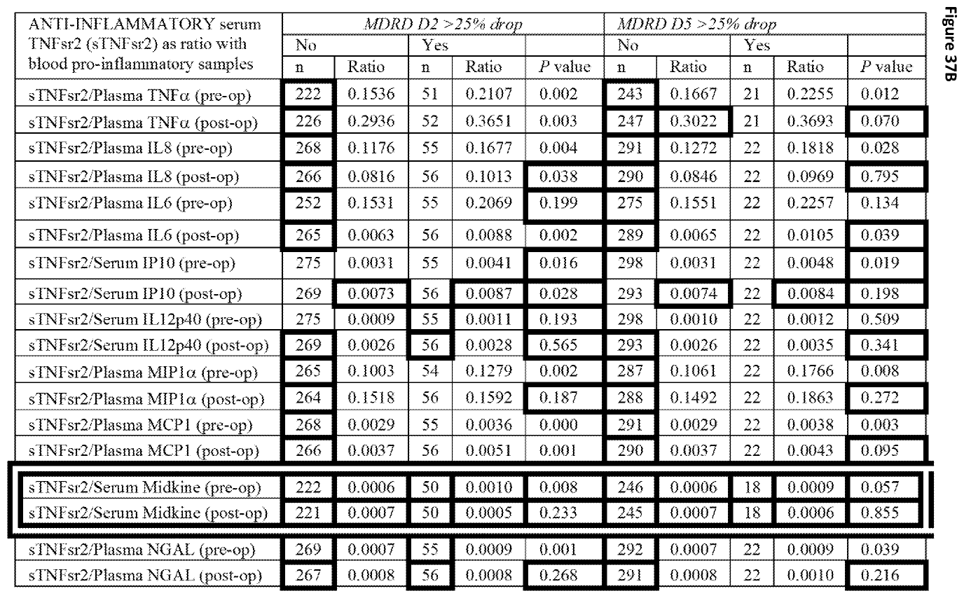

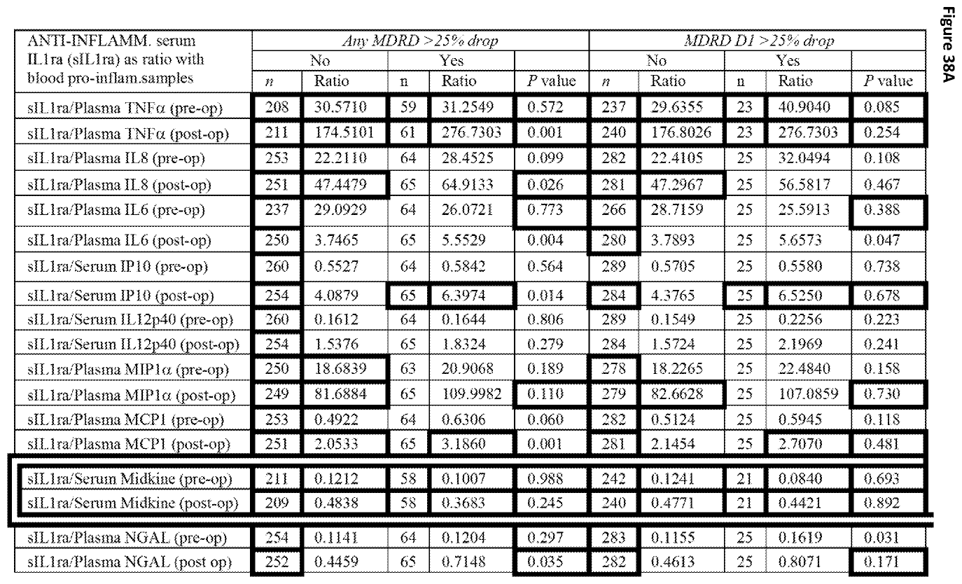

FIG. 37 illustrates biomarker results;

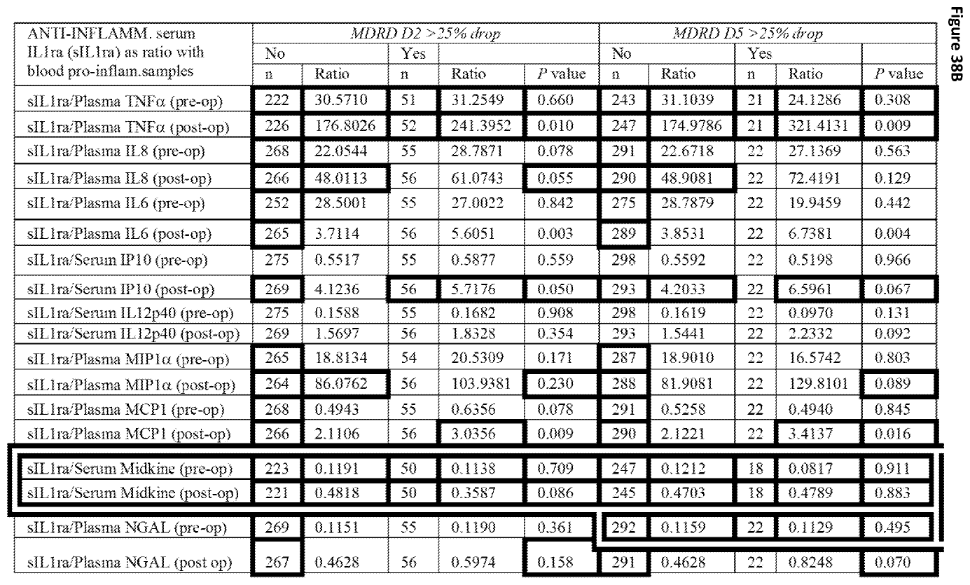

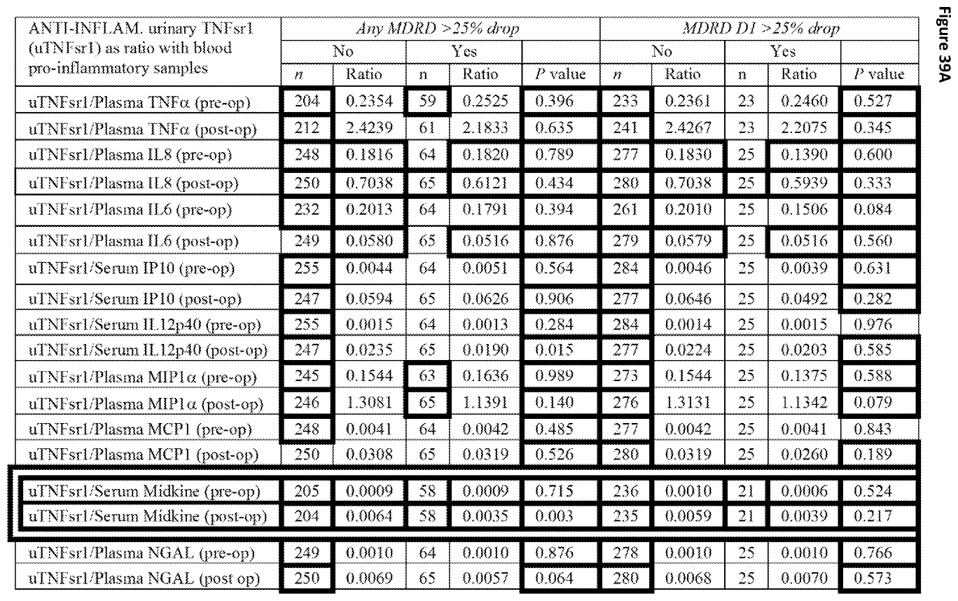

FIG. 38 illustrates biomarker results;

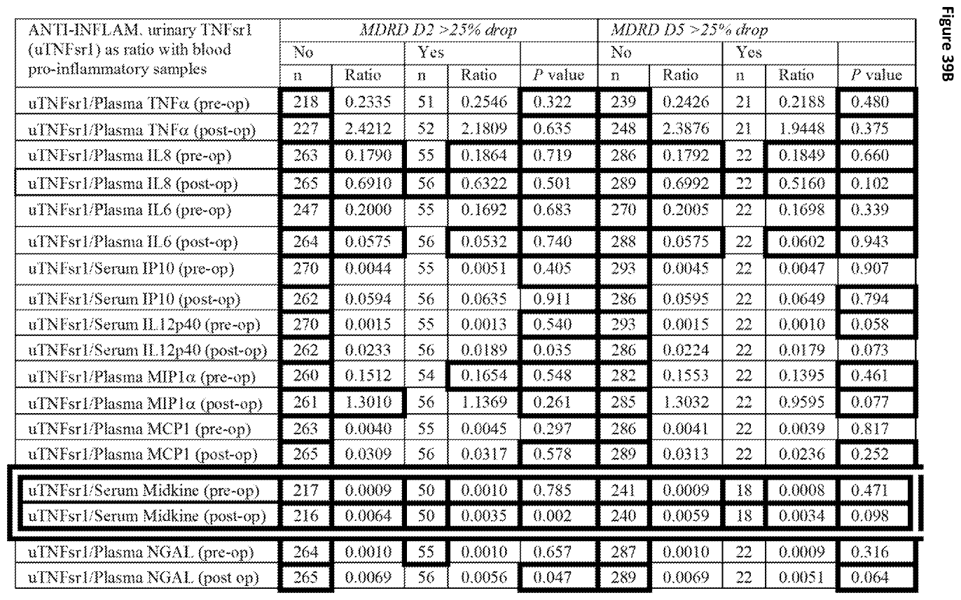

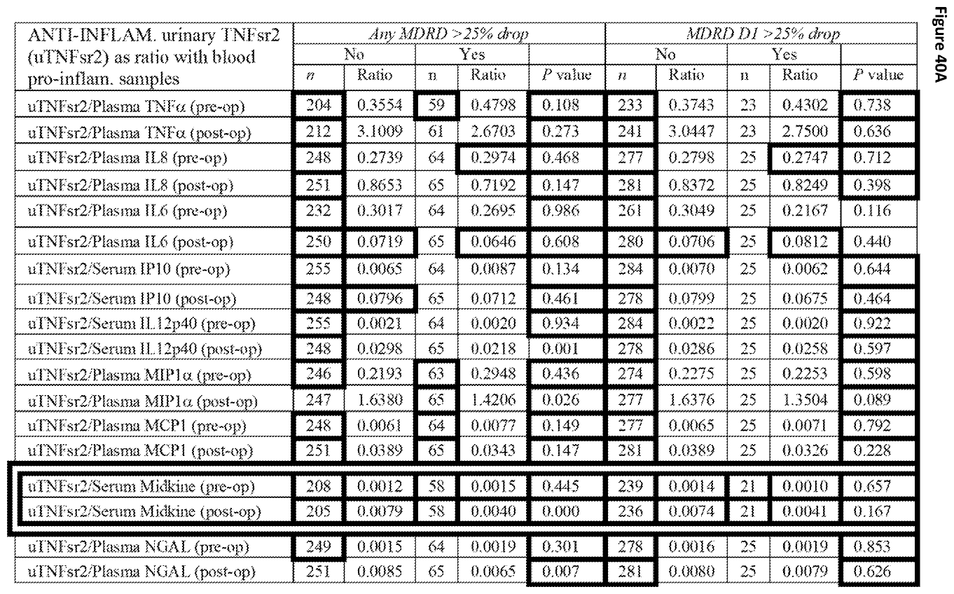

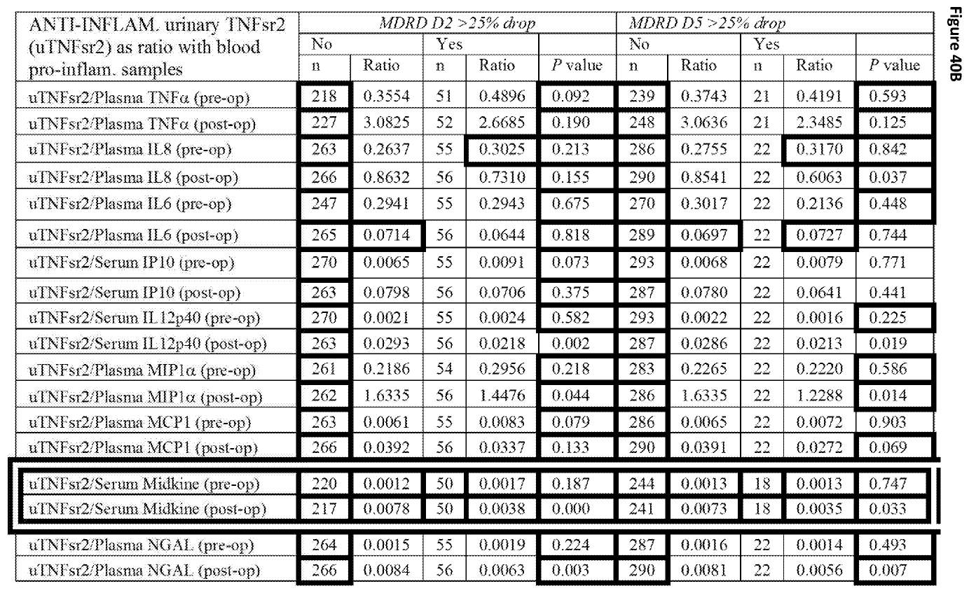

FIG. 39 illustrates biomarker results;

FIG. 40 illustrates biomarker results;

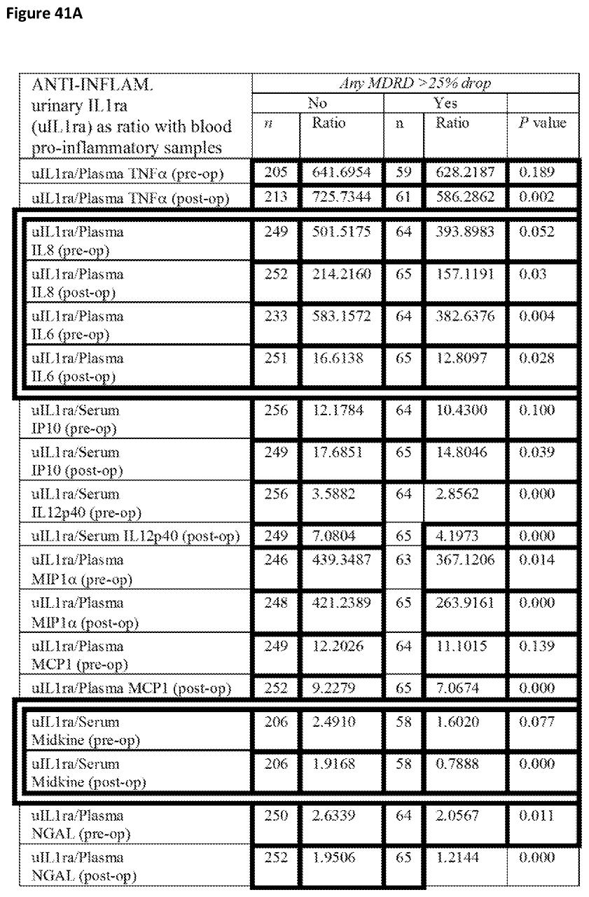

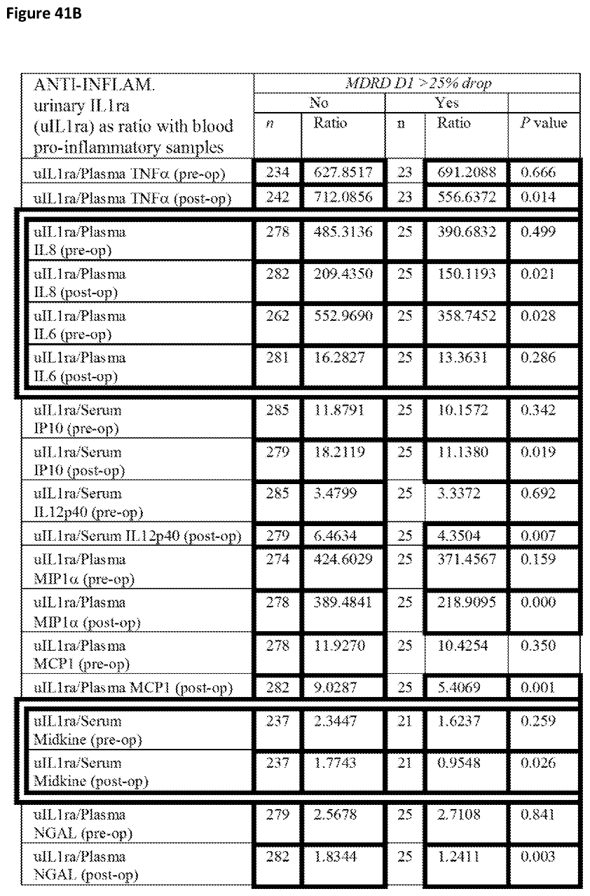

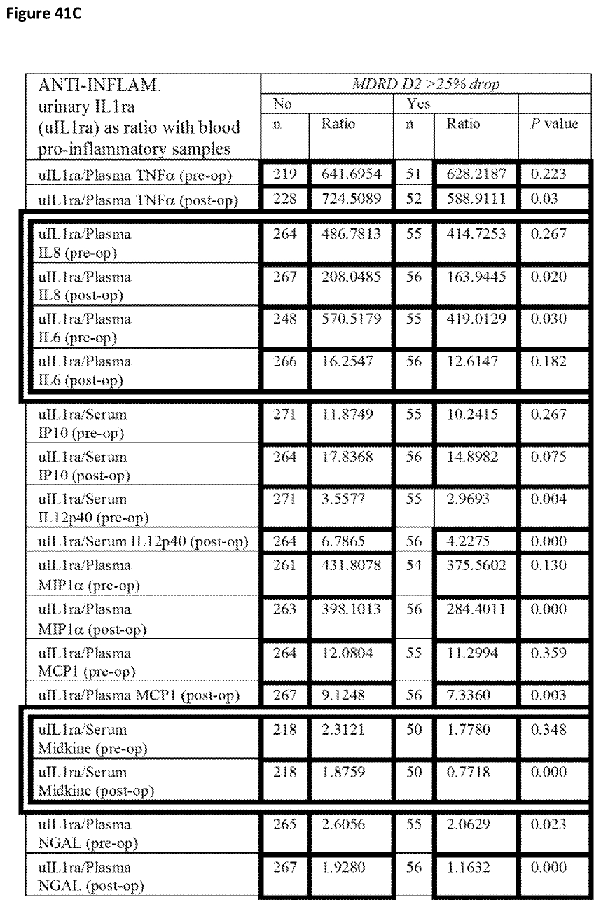

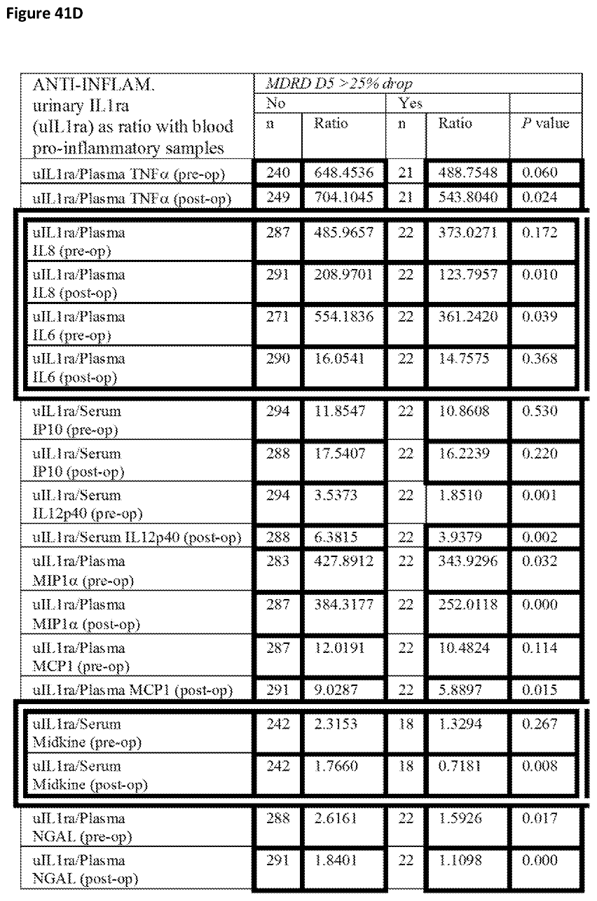

FIG. 41 illustrates biomarker results;

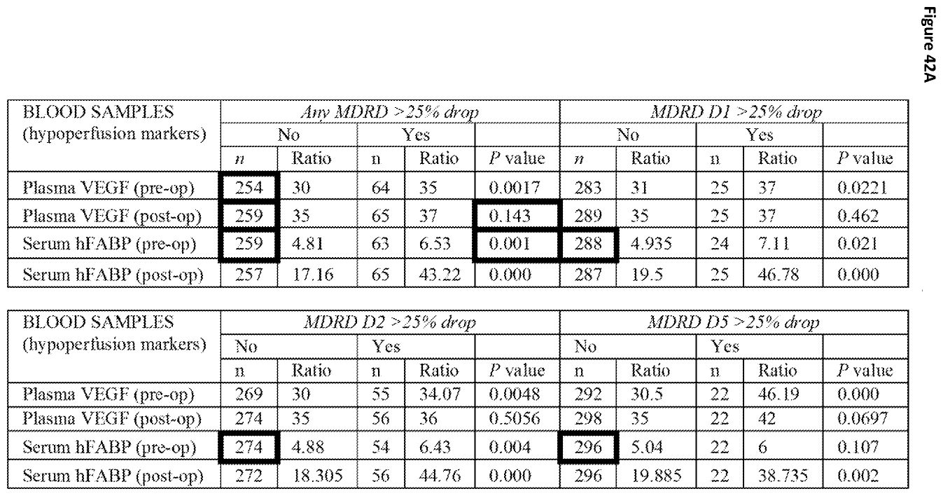

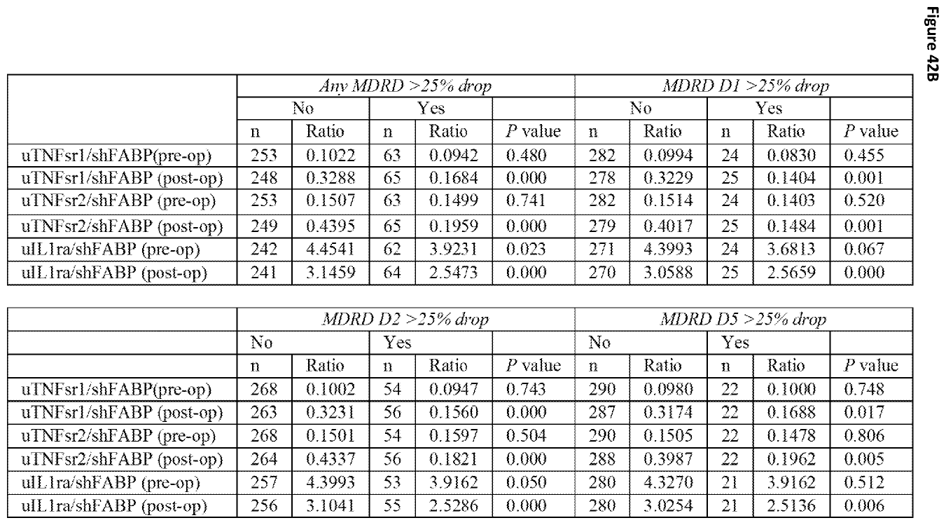

FIG. 42 illustrates biomarker results;

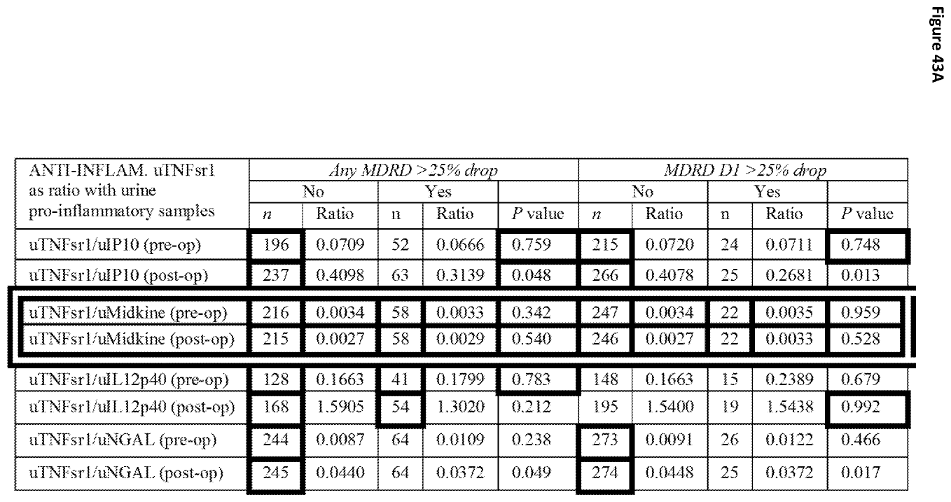

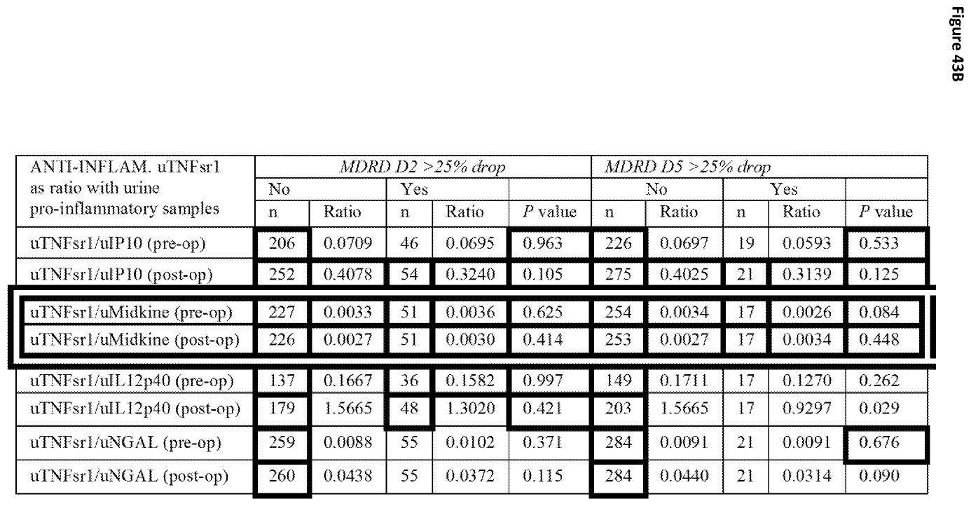

FIG. 43 illustrates biomarker results;

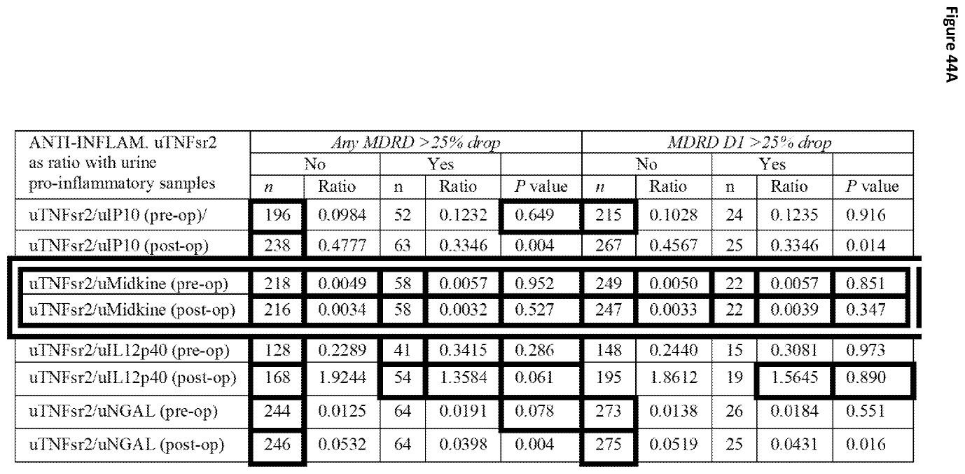

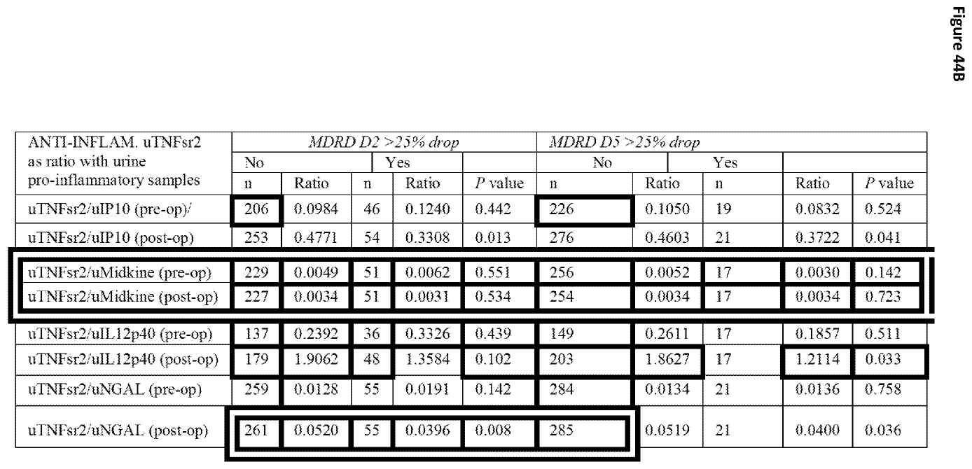

FIG. 44 illustrates biomarker results; and

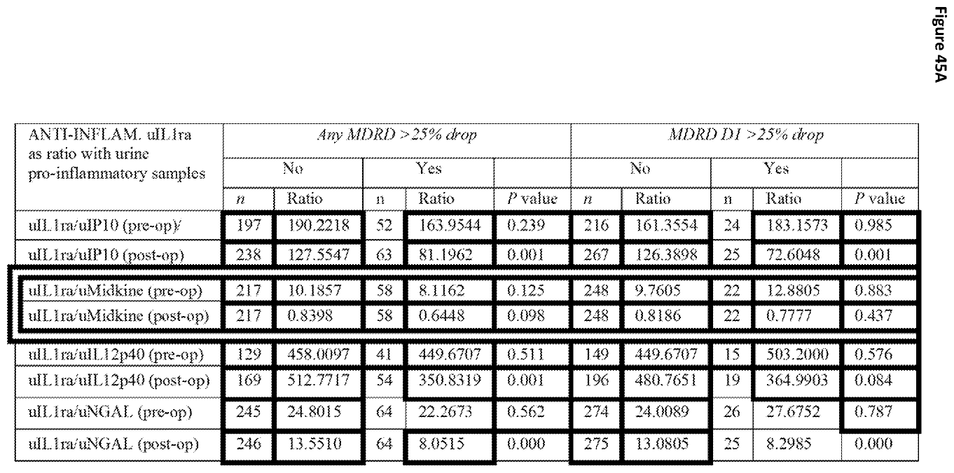

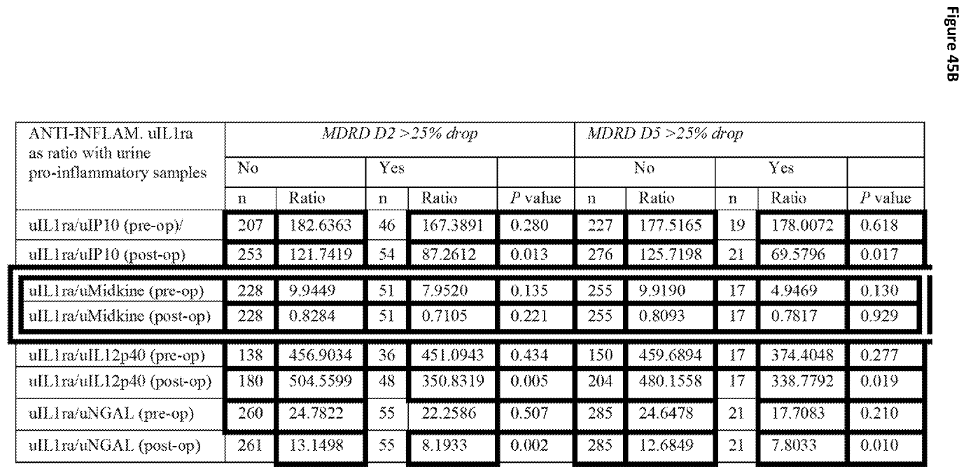

FIG. 45 illustrates biomarker results.

### EXPERIMENTAL METHODS

Ethical approval was granted from the Research Ethics Committee and the Royal Research Office research governance committee.

Cardiac surgery patients were consecutively scheduled for elective and emergency cardiac surgery within the Cardiac Surgical Unit of the Royal Victoria Hospital Belfast while the orthopaedic trauma patients were consecutively scheduled for open reduction internal fixation of their fracture within the fracture unit of the Belfast Trust. Exclusion criteria for all patients was pre-operative or pre-trauma dialysis dependent renal failure or known significant renal disease prior to entrance into the study (known eGFR <30).

### Sampling Protocol

Urinalysis (Sample A) (20 ml) was performed on admission (for fracture patients) or for cardiac patients following catheterisation on induction of anaesthesia and (Sample B) (20 ml) on day 1 post-operatively.

Blood Sample A (20 ml) was performed on admission for fracture patients together with their routine pre-operative blood sample work-up such that participation in this study did not involve additional venepuncture. Blood sample A for cardiac patients followed routine arterial line insertion pre-operatively. Day 1 post-operative Sample B (20 ml) for fracture patients was taken at the time of routine analyses, hence not requiring further venepuncture. If, for a fracture patient, a blood sample was inadvertently missed during the time that routine bloods are being taken, it was not pursued to avoid additional discomfort of venepuncture beyond what was required for routine care, unless that patient, in intensive care or HDU had a routinely placed arterial catheter in situ. Unlike many fracture patients who do not have a routinely placed arterial line, all cardiac surgery patients have an arterial line inserted pre-operatively, remaining in situ for 48 hours such that obtaining the post-operative day 1 blood Sample B will be painless.

Blood and urine samples were immediately centrifuged in the clinical area and the

TABLE 1
AKI is defined as a drop of eGFR >25% from baseline. The number of
patients in each group is shown for each of the AKI time points.

| | Non-AKI patients | AKI patients |
|---|---|---|
| AKI Day 2 | 220 | 50 |
| AKI Day 5 | 246 | 18 |
| AKI Any Day | 210 | 58 |

resulting supernatants stored in fridges. Each week these samples were transported to Randox Laboratories Ltd for storage and analysis.

### EXAMPLE 1

### Cardiac Surgery

Acute Kidney Injury (AKI) was defined as a drop of baseline eGFR of >25%. A drop in eGFR was recorded on day 1, day 2 and day 5 following surgery. To increase the number of patients in the population, the analyses are based upon an ‘AKI any-day’ definition. Patients were included in this category if eGFR dropped to less than 75% of baseline, at any day following cardiac surgery. Based on this criteria, a population was described.

The levels of biomarkers were then determined from the AKI and non-subjects for for plasma, serum and urinary samples. Biomarkers were highlighted as significant at p<0.05 between the AKI and non-AKI groups using a Mann-Whitney U test. The predictive ability of individual biomarkers, identified as significant by Mann-Whitney U test, was investigated by ROC analyses.

Results for plasma, serum and urinary biomarkers are shown in Tables 2, 3 and 4, respectively.

TABLE 2
Plasma biomarkers identified as showing a significant difference between
AKI and non-AKI.

| Marker | Pre/Post surgery | Sig. | AUC |
|---|---|---|---|
| IL-6 | Pre | 0.001 | 0.630 |
| IL-1a | Pre | 0.040 | 0.628 |
| VEGF | Pre | 0.002 | 0.628 |
| INFy | Pre | 0.030 | 0.550 |
| TNFα | Pre and Post | <0.001, <0.001 | 0.645 |
| MCP | Pre and Post | 0.010, 0.001 | 0.604 |
| MIP1α | Pre and Post | 0.001, <0.001 | 0.639 |
| NGAL | Pre and Post | 0.001, <0.001 | 0.632 |
| IL-8 | Post | 0.005 | 0.614 |
| IL-10 | Post | 0.009 | 0.604 |

TABLE 3
Serum biomarkers identified as showing a significant difference between
AKI and non-AKI.

| Marker | Pre/Post surgery | Sig. | AUC |
|---|---|---|---|
| IL12P40 | Pre and Post | <0.001, <0.001 | 0.682, 0.714 |
| IL1RA | Pre and Post | 0.005, <0.001 | 0.542, 0.687 |
| IP10 | Pre and Post | 0.012, 0.046 | 0.601, 0.580 |
| TNFRI | Pre and Post | <0.001, <0.001 | 0.748, 0.748 |
| TNFRII | Pre and Post | <0.001, <0.001 | 0.715, 0.763 |
| H-FABP | Pre and Post | 0.001, 0.001 | 0.637, 0.637 |
| Midkine | Post | <0.001 | 0.695 |

TABLE 4
Urine biomarkers identified as showing a significant difference
between AKI and non-AKI.

| | Pre/Post | | |
|---|---|---|---|
| Marker | Surgery | Sig. | AUC |
| TNFRI | Pre and Post | 0.044, 0.020 | 0.581, 0.593 |
| TNFRII | Pre and Post | 0.018, 0.018 | 0.595, 0.595 |
| IL12P40 | Pre and Post | 0.035, <0.001 | 0.580, 0.667 |
| IL10 | Post | 0.001 | 0.637, 0.637 |
| IL1Ra | Post | 0.024 | 0.591 |
| NGAL | Post | <0.001 | 0.651 |

Biomarker combinations were then considered which provided the greatest ability to predict AKI from non-AKI subject groups.

### EXAMPLE 2

### Fracture Surgery

AKI could not be defined using the same definition as applied in the cardiac population, namely a drop of >25% in eGFR from baseline, as patients may already have AKI, as a result of their fracture injury, before surgery. The Classification of AKI in the fracture population was based on the RIFLE system. Patients were assumed to have a baseline GFR of at least 60m1/min/1.73m² therefore, a value of less than 45m1/min/1.73m² was used to define a patient as AKI positive.

The levels of biomarkers were then determined from the AKI and non-subjects for for plasma, serum and urinary samples. Biomarkers were highlighted as significant at p<0.05 between the AKI and non-AKI groups using a Mann-Whitney U test. The predictive ability of individual biomarkers, identified as significant by Mann-Whitney U test, was investigated by ROC analyses.

Results for plasma, serum and urinary biomarkers are shown in Tables 5, 6 and 7, respectively.

TABLE 5
Plasma biomarkers identified as showing a significant
difference between AKI and non-AKI group.

[symbol]

[symbol]

indicates data missing or illegible when filed

TABLE 6
Serum biomarkers identified as showing a statistically significant difference
between AKI and non-AKI groups.

| Marker | Pre/Post Surgery | Sig. | AUC |
|---|---|---|---|
| IL12P40 | Pre and Post-surgery | 0.004, 0.021 | 0.637, 0.619 |
| IL1RA | Post-surgery | 0.003 | 0.653 |
| TNFRI | Pre and Post-surgery | <0.001 | 0.729, 0.795 |
| TNFRII | Pre and Post-surgery | 0.004, <0.001 | 0.634, 0.734 |
| IL2Ra | Post-surgery | 0.040 | 0.606 |
| H-FABP | Pre and Post-surgery | <0.001 | 0.712, 0.829 |
| Midkine | Pre and Post-surgery | 0.035, 0.001 | 0.615, 0.678 |
| IL-6 | Post-surgery | 0.001 | 0.661 |
| TNFα | Pre and Post-surgery | 0.02, 0.002 | 0.607, 0.653 |
| MCP | Pre and Post-surgery | 0.035, 0.003 | 0.597, 0.646 |
| IL-5 | Pre-surgery | 0.039 | 0.594 |
| IL-15 | Post-surgery | 0.001 | 0.523, 0.663 |
| MIP1α | Pre and Post-surgery | 0.009, 0.003 | 0.619, 0.644 |
| NGAL | Pre and Post-surgery | <0.001, <0.001 | 0.752, 0.812 |

TABLE 7
Urinary biomarkers identified as significant between AKI and non-AKI
groups.

| Marker | Pre/Post Surgery | Sig. | AUC |
|---|---|---|---|
| TNFRI | Pre and Post-Surgery | 0.004, 0.001 | 0.638, 0.656 |
| NGAL | Post-surgery | 0.008 | 0.631 |

Biomarker combinations were then considered which providing the greatest ability to predict AKI from non-AKI subject groups.

Further, analysis of biomarkers was undertaken wherein ratios between biomarkers were considered. Ratios of urinary anti- and pro-inflammatory mediators were determined. Further ratios of anti- and pro-inflammatory mediators in the blood (in particular the serum) were considered. Additionally, ratios of anti- and pro-inflammatory mediators in the urine and blood were considered.

Based on this work it was considered that the ratio or balance of anti- and pro-inflammatory mediators in blood and urine will be lower in those subjects that develop renal dysfunction post operatively that those subjects retaining normal post-operative renal function.

Without wishing to be bound by theory, it is considered that in blood and urine of those subjects who develop renal dysfunction (AKI) there would either be no correlation or a negative correlation between anti- and pro-inflammatory mediators. Further it is considered that hypoperfusion in combination with a further insult of an imbalanced inflammatory response can be more injurious to the kidney than either insult occurring alone. Since hypoperfusion is an important contributing factor to perioperative AKI, suitably markers such as HFABP and VEGF can be measured and used to assess AKI risk.

Based on the results shown in FIGS. 30 to 45 (wherein no shading indicates no differences in the Number of subjects (n) between SPSS and PRISM results/no differences or small differences in Ratios and p-values between SPSS and PRISM results and shading indicates differences in the Number of subjects (n) between SPSS and PRISM results/Greater differences in Ratios and p-values between SPSS and PRISM results, particular ratios were found to be predictive.

It was determined that the ratio of postoperative urinary TNFsr2/serum HFABP fell in AKI patients, suggesting that organ hypoperfusion as indicated by increased serum HFABP was particularly injurious to those patients who did not develop an adequate anti-inflammatory urinary TNFsr2 response. Without wishing to be bound by theory it is considered that an inadequate protective urinary anti-inflammatory response becomes significant in those patients who endure the double insults of proinflammation (as evidenced by urinary proinflammatory mediators) as well as hypoperfusion (as evidenced by increased serum HFABP). It is further considered that inadequate protective urinary anti-inflammatory response becomes significant in those patients who endure the double insults of proinflammation (as evidenced by blood and urinary proinflammatory mediators)as well as hypoperfusion as evidenced by increased serum HFABP and VEGF.

Preoperative VEGF has been determined to be higher in the AKI group for all endpoints including D5 renal dysfunction—(P<0.001). VEGF is considered to be a direct hypoperfusion/hypoxia marker.

Without wishing to be bound by theory, it is considered that post operatively, after cardiac surgery, when the heart is fixed, overall perfusion and oxygenation is typically improved. HFABP post op does not typically indicate overall oxygenation and perfusion post op. Post op HFABP may be considered to be more indicative of the magnitude of the intraoperative insult and in turn be predictive of AKI.

Post op hypoperfusion/hypoxia is typically less of a problem in the majority of straightforward low risk cases because following cardiac surgery the heart is revascularised and the subject is typically mechanically ventilated with oxygen supplementation giving them a supra physiological pO2. VEGF as a marker of hypoperfusion and hypoxia at the moment of measurement, may those be indicative of artificially supplemented oxygenation. However, a rise in VEGF post op may be particularly significant.

Pre op VEGF thus is considered predictive of vulnerability to perioperative hypoperfusion on CPB with heightened AKI risk stretching as far as D5.

In fracture subjects it was determined that peri-operative increases were observed in almost all blood pro- and anti-inflammatory mediators measured, with some pro-inflammatory mediators (pre and post op) and some anti-inflammatory mediators showing significantly greater increases in the renal dysfunction group compared with the normal renal function subjects.

It is considered results determined in relation to cardiac surgery with respect to pre and post operative blood and urinary cytokines and anti/pro inflammatory cytokine ratios are similarly observed in post trauma orthopaedic surgery patients with the difference that base line post trauma pre operative samples will have undergone some increase in response to the fracture trauma.

For fracture subjects, it is considered the ratio of

urinary TNFsr1/HFABP (serum)
TNFsr1(UA)/HFABP (SA) (Do AKI) (was lower in AKI)
TNFsr1 (UB)/HFABP (SB) (D0,D1, D5) (was lower in AKI)
   urinary TNFsr2/HFABP(serum)

TNFsr2(UA)/HFABP (SA) (Do AKI) (was lower in AKI)
TNFsr2 (UB)/HFABP(SB) (D0, D1,D2,D5) (was lower in AKI)

UA refers to ‘pre’ urine samples and UB refers to ‘post’ urine samples. The same applies for serum.

For the fracture patients—D0=before the operation (this can still be 3-4 days after the initial trauma)
D1 =24 hours after the operation
D5 =5 days after the operation and
TNFsr2UB/TNFSR2 (SB) (D5AKI) (was lower in AKI) are particularly significant.

This has also been considered in cardiac patients wherein the ratio of postoperative urinary TNFsr2/serum HFABP fell in AKI patients, suggesting that organ hypoperfusion as indicated by increased serum HFABP was particularly injurious to those patients who did not develop an adequate anti-inflammatory urinary TNFsr2 response.

Post-operative ratios of urinary anti-inflammatory mediators/blood pro-inflammatory mediators were generally lower in renal dysfunction patients than the normal renal patients.

Further it is considered increased TNFa in blood correlates weakly but significantly with TNFsr2 in urine in non-AKI patients. This may suggest that in AKI patients the post operative compensatory rise in urinary TNFsr2 is compromised: and there may be a tendency to this deficiency pre-operatively even when renal function is normal

TABLE 8
Plasma TNFα vs urinary TNFsr2 - cardiac

| | TNFSR2 UB/HFABP SB | | | | |
|---|---|---|---|---|---|
| | AKI | AKI | | Normal | Normal |
| | N | Median | N | Median | P Value |
| D1 | 25 | 0.1484 | 286 | 0.3978 | 0.0016 |
| D2 | 56 | 0.1821 | 271 | 0.4061 | <0.001 |
| DS | 22 | 0.1962 | 295 | 0.3949 | 0.0102 |
| Any time | 65 | 0.1959 | 247 | 0.4395 | <0.0001 |

TABLE 10

| Timepoint | | P-Value | r-Value | n (XY pairs) |
|---|---|---|---|---|
| A | AKI Anytime 75% | 0.6016 | 0.0665 | 64 |
| | Non-AKI | 0.0045 | 0.1834 | 239 |
| B | AKI Anytime 75% | 0.6387 | 0.05933 | 65 |
| | Non-AKI | <0.0001 | 0.2553 | 242 |

Correlations between plasma TNFα and urinary TNFsr2

TABLE 11
TNFSR2 UA/HFABP SA

| | AKI | | Non AKI | | |
|---|---|---|---|---|---|
| | N | Mean | N | Mean | P Value |
| Any Time AKI | 63 | 0.1441 | 250 | 0.1384 | 0.5313 |
| Day 1 AKI | 24 | 0.1403 | 288 | 0.1482 | 0.6467 |
| Day 2 AKI | 54 | 0.1597 | 274 | 0.1395 | 0.3645 |
| Day 5 AKI | 22 | 0.1478 | 296 | 0.1442 | 0.9358 |

TABLE 12
TNFSR2 UB/HFABP SB

| | SB | | | | |
|---|---|---|---|---|---|
| | AKI | AKI | Normal | Normal | |
| | N | Median | N | Median | P Value |
| D1 | 25 | 0.1484 | 286 | 0.3978 | 0.0016 |
| D2 | 56 | 0.1821 | 271 | 0.4061 | <0.001 |
| D5 | 22 | 0.1962 | 295 | 0.3949 | 0.0102 |
| Anytime | 65 | 0.1959 | 247 | 0.4395 | <0.0001 |

In the cardiac study it was determined that there were peri-operative increases in almost all blood pro- and anti-inflammatory mediators measured, with both pro-inflammatory mediators (pre and post op TNFalpha, IP10, IL12p40, MIP1alpha, MCP1 and NGAL; post op IL8 and Midkine; pre op IL6) and anti-inflammatory mediators (pre and post op TNFsr1, TNFsr2 and IL1ra; post op IL10) showing significantly greater increases in the anytime renal dysfunction group compared with the normal renal function patients. This study shows that in comparison with those patients who retained normal renal function, those who developed renal dysfunction post operatively had a higher baseline preoperative blood concentration of a range of pro-inflammatory markers (eg TNFa, IP10, IL12p40, MIP1alpha, MCP1, NGAL and 1L6) as well as a greater postoperative proinflammatory response in blood (eg TNFa, IP10, IL12p40, MIP1alpha, MCP1, NGAL, IL8, and Midkine).

Determination of ratios of blood anti-inflammatory mediators/blood pro-inflammatory mediators consistently showed higher anti-inflammatory/pro-inflammatory ratios in the renal dysfunction group(these differences being significant in pre and post op sTNFsr1/plasma TNF, sTNFsr1/plasmaMCP1, sTNFsr2/plasma TNFalpha, sTNFsr2/plasma IL8, sTNFsr2/sIP10, sTNFsr2/plasmaMCP1; pre-op sTNFsr1/plasma IL8, sTNFsr1/sIP10, sTNFsr1/plasma MIP1alpha, sTNFsr1/plasma NGAL, sTNFsr2/plasma MIP1alpha, sTNFsr2/sMidkine, sTNFsr2/plasmaNGAL; post op sTNFsr1/plasma IL6, sTNFsr2/plasma IL6, sIL1ra/plasma TNFalpha, sIL1ra/plasma IL8, sIL1ra/plasma IL6, sIL1ra/sIP10, sIL1ra/plasma MCP1, sIL1ra/plasma NGAL).

In cardiac and fracture studies, in contrast to blood, in the urine, the anti-/pro-inflammatory ratios were consistently lower in the renal dysfunction patients than the normal renal function patients. In the cardiac study, these differences were significant with respect to any time renal dysfunction in the following ratios: post- operative uTNFsr1/uIP10; post-op uTNFsr1/uNGAL; post-operative uTNFsr2/uIP10; post-operative uTNFsr2/uNGAL; post-operative uIL1ra/uI1P10; post-operative ulL1ra/uIL12p40; post-operative uIL1ra/uNGAL. These differences were also significant with respect to D5 renal dysfunction patients in the following ratios: post- operative uTNFsr2/uIL12p40; post-operative uIL1ra/uIP10; post-operative uIL1ra/uIL12p40; post-operative uIL1ra/uNGAL.

Post-operative ratios of urinary anti-inflammatory mediators/blood pro-inflammatory mediators were generally lower in the renal dysfunction patients than the normal renal function patients. Differences considered to be significant with respect to any time renal dysfunction were the following post-operative ratios: uTNFsr1/sIL12p40, uTNFsr1/sMidkine, uTNFsr2/sIL12p40, uTNFsr2/pMIP1alpha, uTNFsr2/sMidkine, uTNFsr2/pNGAL, uIL1ra/pTNFalpha, uIL1ra/pIL8, uIL1ra/pIL6, uIL1ra/sIP10, uIL1ra/sIL12p40, uIL1ra/pMIP1alpha, uIL1ra/pMCP1, uIL1ra/sMidkine, uIL1ra/pNGAL.

Although no pre-operative uTNFSR1/blood pro-inflammatory or uTNFsr2/blood pro-inflammatory ratios were different between normal renal function and renal dysfunction groups, some pre-operative uIL1ra/blood pro-inflammatory ratios were also significantly lower in those who later developed renal dysfunction namely preoperative uIL1ra/pIL6, uIL1ra/sIL12p40, uIL1ra/pMIP1alpha, uIL1ra/pNGAL. Of particular interest to clinicians, these differences were also significant with respect to D5 renal dysfunction patients in the following post operative ratios: post op uTNFsr2/plL8, uTNFsr2/sIL12p40, uTNFsr2/pMIP1alpha, uTNFsr2/pNGAL, uIL1ra/pTNFalpha, uIL1ra/pIL8, uIL1ra/sIL12p40, uIL1ra/pMIP1alpha, uIL1ra/pMCP1, uIL1ra/sMidkine, uIL1ra/pNGAL and of note with respect to the pre-operative ratio of pre-operative uIL1ra/pIL6, uIL1ra/sIL12p40, uIL1ra/pMIP1alpha, uIL1ra/pNGAL.

If urinary anti-inflammatory/blood pro-inflammatory ratios (in contrast to blood anti-inflammatory/blood pro-inflammatory ratios), are lower in the renal dysfunction groups than in the normal renal function subjects, it may be inferred that filtered blood pro-inflammatory mediators are less well counterbalanced by a compensatory intrarenal anti-inflammatory response in the renal dysfunction patients than the normal renal function patients. This is further corroborated by the demonstration of negative correlations between blood pro- and urinary anti-inflammatory mediators in the renal dysfunction groups.

### Ratio of Urinary TNFsr2/Serum HFABP

Finally, in cardiac patients, the ratio of postoperative urinary TNFsr2/serum HFABP fell in AKI patients, suggesting that organ hypoperfusion as indicated by increased serum HFABP was particularly injurious to those patients who did not develop an adequate anti-inflammatory urinary TNFsr2 response.

Each document, reference, patent application or patent cited in this text is expressly incorporated herein in their entirety by reference, which means it should be read and considered by the reader as part of this text. That the document, reference, patent application or patent cited in the text is not repeated in this text is merely for reasons of conciseness.

Reference to cited material or information contained in the text should not be understood as a concession that the material or information was part of the common general knowledge or was known in any country.

Although the invention has been particularly shown and described with reference to particular examples, it will be understood by those skilled in the art that various changes in the form and details may be made therein without departing from the scope of the present invention.

1. A method to determine a predisposition of a subject to developing AKI, the method comprising the step of:
determining the level of at least one marker selected from Midkine (MK) or H-FABP present in a blood or urine sample taken from the subject within 48 hours following surgery, physical trauma, hypotension, sepsis and/or septic shock syndrome, in particular cardiac surgery or a fracture trauma;
wherein when the level of the Midkine or H-FABP is higher than a normal level of Midkine or H-FABP in a blood or urine sample from a control it is indicative the subject has a greater than normal predisposition for developing AKI following surgery, physical trauma, hypotension, sepsis and/or septic shock syndrome, in particular cardiac surgery or a fracture trauma.

2. A method to determine a predisposition of a subject to developing AKI, the method comprising the step of:
determining the level of at least one marker selected from H-FABP or Midkine (MK) present in a blood or urine sample taken from the subject prior to surgery, physical trauma, hypotension, sepsis and/or septic shock syndrome, in particular cardiac surgery or a fracture trauma;
wherein when the level of the H-FABP or Midkine marker is higher than a normal level of H-FABP or Midkine in a blood or urine sample from a control it is indicative the subject has a greater than normal predisposition for developing AKI following surgery, physical trauma, hypotension, sepsis and/or septic shock syndrome, in particular cardiac surgery or a fracture trauma.

3. The method of claim 1 or 2 wherein the step of determining the level of at least one marker selected from H-FABP or Midkine is undertaken on serum from the subject.

4. The method of any previous claim wherein the determining step comprises determining the level of at least two markers wherein a first marker is selected from at least one of Midkine (MK) and H-FABP and at least a second marker is selected from at least one of TNFRII and TNFRI wherein when the detected level of TNFRII and/or TNFRI is higher than a normal level of TNFRII and/or TNFRI respectively in a control it is indicative the subject has a greater than normal predisposition for developing AKI following cardiac surgery or fracture trauma.

5. The method of any previous claim wherein the determining step comprises determining the level of at least three markers selected from TNFRI, TNFRII and Midkine.

6. The method of any of claims 1 to 4 wherein the determining step comprises determining the level of at least three markers selected from TNFRI, TNFRII and H-FABP.

7. The method of any of claims 1 to 4 wherein the determining step comprises determining the level of at least three markers selected from TNFRI, H-FABP and Midkine.

8. The method of any previous claim wherein the method further comprises detecting at least one marker selected from IL-1a, IL-5, IL-6, IL-8, IL-10, IL-15, MIP1-alpha, VEGF, INF-gamma, TNF-alpha, MCP, NGAL, IL12P40, IP10 or IL1Ra.

9. The method of claim 8 wherein at least one of IL-1a, IL-5, IL-6, IL-8, IL-10, IL-15, MIP1-alpha VEGF, INF-gamma, TNF-alpha, MCP and NGAL is detected from plasma from the subject.

10. The method of claim 8 wherein IL12P40 is detected from serum or urine of a subject.

11. The method of claim 8 wherein IP10 or IL1Ra are detected in urine.

12. The method of any of claims 2 to 11 wherein the sample is obtained from a subject within 24 hours of a proposed surgery.

13. The method of any of claims 1 and 3 to 11 wherein the sample is obtained from a subject within 24 hours post surgery.

14. The method of any preceding claims wherein the sample is plasma or serum or urine.

15. The method of any of the previous claims wherein a ratio of urinary TNFsr1/HFABP (serum) is determined wherein TNFsr1 (UB)/HFABP (SB) is lower in AKI subjects than non AKI subjects.

16. The method of any of the previous claims wherein the ratio of urinary TNFsr2/HFABP(serum) is determined wherein TNFsr2 (UB)/HFABP(SB) is lower in AKI subjects than non AKI subjects.

17. The method of any of claims 1 to 14 wherein the method comprises determining AKI risk on post-operative ratios selected from uTNFsr1/sMidkine, uTNFsr2/sMidkine or ulL1ra/sMidkine wherein the urinary anti-inflammatory/blood pro-inflammatory ratios are lower in those who develop renal dysfunction.

18. The method of any of claims 1 to 14 wherein the method comprises determining pre-operative uTNFSR1/blood pro-inflammatory or uTNFsr2/blood pro-inflammatory ratios wherein urinary anti-inflammatory/blood pro-inflammatory ratios were lower in those who later developed renal dysfunction.

19. A kit for use in the method of any preceding claim, wherein the kit comprises:-
one or more reagents to detect at least one of TN FI, TNFII, Midkine and H-FABP or a combination thereof
instructions for determining whether the at least one of TNFRI, TNFRII, Midkine and H-FABP is higher than a normal level as observed in a subject according to the first or second aspect.

20. A method of treating renal dysfunction induced by surgery, physical trauma, hypotension, sepsis and/or septic shock syndrome, in particular cardiac surgery or a fracture trauma;
wherein the method includes the steps of: (i) prognosing renal dysfunction according to any of the methods of claims 1 to 19; and (ii) when the subject is identified to be at increased risk of developing renal dysfunction, applying therapeutic measures to treat or obviate the impending renal dysfunction.

21. The method of claim 20 wherein the therapeutic measures applied in step (ii) are selected from: maintaining a supra-normal blood pressure; ensuring adequate tissue oxygen delivery; administration of steroids; renal replacement therapy; dialysis; or any combination thereof, administration of erythropoietin, minimizing the duration of cardiopulmonary bypass, early renal replacement therapy.
